(19) United States
(12) Patent Application Publication
(10) Pub. No.: US 2026/0256941 A1
(43) Pub. Date: Sep. 3, 2026

(54) ENGINEERED EXTRACELLULAR VESICLES FOR THERAPEUTIC DELIVERY

(71) Applicant: TELOS THERAPEUTICS, INC., wilmington, DE (US)

(72) Inventors: Charles Cameron TAYLOR, Irvine, CA (US); Milad Riazifar, Irvine, CA (US); Todd SCHURR, Irvine, CA (US)

(21) Appl. No.: 18/877,707

(22) PCT Filed: Jun. 22, 2023
(86) PCT No.: PCT/US2023/068911

§ 371 Date: Dec. 20, 2024

Related U.S. Application Data
(60) Provisional application No. 63/354,621, filed on Jun. 22, 2022.
(60) Provisional application No. 63/354,624, filed on Jun. 22, 2022.

(51) Int. Cl. A61K 47/69; A61K 47/62; A61P 25/28; A61P 37/06

(52) U.S. Cl. CPC A61K 47/6901; A61K 47/62; A61P 25/28; A61P 37/06

(57) ABSTRACT

Extracellular vesicles comprising a bi-layer lipid membrane comprising an inner layer and an outer layer and a first recombinant protein comprising a first transmembrane moiety embedded between the inner layer and the outer layer of the lipid membrane, and a first affinity receiver located on or outside the outer layer of the lipid membrane, wherein the affinity receiver binds an affinity tag on a messenger protein.

### CROSS-REFERENCE

This application claims the benefit of U.S. Provisional Application No. 63/354,624, filed on Jun. 22, 2022 and U.S. Provisional Application No. 63/354,621, filed on Jun. 22, 2022, each of which are herein incorporated by reference in their entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Extracellular vesicles (EVs) are small membranous particles that can mediate intercellular communication and could be used as carriers for delivering therapeutics to a target cell for treating diseases or disorders. There are three categories of EVs based on their subcellular origin and size: exosomes, microvesicles, and apoptotic bodies. Exosomes (e.g., 30-150 nm) contain membrane-associated lipids, proteins, and RNA cargoes. Exosome-specific or -selective proteins are proteins that may be displayed on exosome membranes, including tetraspanin proteins (e.g. CD81, CD82, CD37, and CD63), Alix, TSG101, Rab5, HSC70, and HSP900 and other proteins involved in cell adhesion and signaling, cytoskeletal structures, lipid rafts, and membrane trafficking.

Exosomes may be associated with immune responses, viral pathogenicity, pregnancy, cardiovascular diseases, central nervous system-related diseases, and cancer progression. Exosomes may be engineered to deliver diverse therapeutic payloads with an ability to direct their delivery to a desired target.

### SUMMARY

Disclosed herein, in some embodiments, is an extracellular vesicle, comprising a bi-layer lipid membrane comprising an inner layer and an outer layer and a first recombinant protein comprising a first transmembrane moiety embedded between the inner layer and the outer layer of the lipid membrane, and a first affinity receiver located on or outside the outer layer of the lipid membrane. In some embodiments, the extracellular vesicle is an exosome. In some embodiments, the first transmembrane moiety comprises at least a portion of an exosome specific protein. In some embodiments, the first affinity receiver comprises a peptide or derivative thereof that is configured to bind to an affinity tag peptide or derivative thereof.

In some embodiments, the first transmembrane moiety comprises at least a portion of IGSF8, LAMP2, CD63, CD81, PTGFR, or a functional derivative thereof. In some embodiments, the first transmembrane moiety comprises a peptide sequence that is at least 50% homologous to IGSF8, LAMP2, CD63, CD81, or PTGFR. In some embodiments, the first transmembrane moiety comprises a peptide sequence that is at least 60% homologous to IGSF8, LAMP2, CD63, CD81, or PTGFR. In some embodiments, the first transmembrane moiety comprises a peptide sequence that is at least 70% homologous to IGSF8, LAMP2, CD63, CD81, or PTGFR. In some embodiments, the first transmembrane moiety comprises a peptide sequence that is at least 80% homologous to IGSF8, LAMP2, CD63, CD81, or PTGFR. In some embodiments, the first transmembrane moiety comprises a peptide sequence that is at least 90% homologous to IGSF8, LAMP2, CD63, CD81, or PTGFR. In some embodiments, the first transmembrane moiety comprises a peptide sequence that is at least 95% homologous to IGSF8, LAMP2, CD63, CD81, or PTGFR.

In some embodiments, the extracellular vesicle further comprises a second recombinant protein comprising a second transmembrane moiety embedded between the inner layer and the outer layer of the lipid membrane, and a second affinity receiver located on or outside the outer layer of the lipid membrane. In some embodiments, the second transmembrane moiety is the same as the first transmembrane moiety, but the second affinity receiver is different from the first affinity receiver. In some embodiments, the second transmembrane moiety is different from the first transmembrane moiety, but the second affinity receiver is the same as the first affinity receiver. In some embodiments, the second transmembrane moiety is the different from the first transmembrane moiety, and the second affinity receiver is different from the first affinity receiver.

In some embodiments, the extracellular vesicle further comprises a third recombinant protein comprising a third transmembrane moiety embedded between the inner layer and the outer layer of the membrane, and a third affinity receiver located on or outside the outer layer of the membrane. In some embodiments, the third transmembrane moiety is the different from the first transmembrane moiety, and the third affinity receiver is different from the first affinity receiver. In some embodiments, the third transmembrane moiety is the same as the first transmembrane moiety, and the third affinity receiver is different from the first affinity receiver. In some embodiments, the third transmembrane moiety is different from the first transmembrane moiety, and the third affinity receiver is the same as the first affinity receiver.

In some embodiments, the first affinity receiver comprises a peptide that is at least 50% homologous to the amino acid sequence ID No. 20. In some embodiments, the first affinity receiver comprises a peptide that is at least 60% homologous to the amino acid sequence ID No. 20. In some embodiments, the first affinity receiver comprises a peptide that is at least 70% homologous to the amino acid sequence ID No. 20. In some embodiments, the first affinity receiver comprises a peptide that is at least 80% homologous to the amino acid sequence ID No. 20. In some embodiments, the first affinity receiver comprises a peptide that is at least 90% homologous to the amino acid sequence ID No. 20. In some embodiments, the first affinity receiver comprises a peptide that is at least 95% homologous to the amino acid sequence ID No. 20. In some embodiments, the first affinity receiver comprises a peptide that is at least 99% homologous to the amino acid sequence ID No. 20. In some embodiments, the first affinity receiver comprises a peptide having the amino acid sequence ID No. 20. In some embodiments, the first affinity receiver comprises a peptide that is at least 50% homologous to SEQ ID No: 21. In some embodiments, the first affinity receiver comprises a peptide that is at least 60% homologous to SEQ ID No: 21. In some embodiments, the first affinity receiver comprises a peptide that is at least 70% homologous to SEQ ID NO: 21. In some embodiments, the first affinity receiver comprises a peptide that is at least 80% homologous to SEQ ID NO: 21. In some embodiments, the first affinity receiver comprises a peptide that is at least 90% homologous to SEQ ID NO: 21. In some embodiments, the first affinity receiver comprises a peptide that is at least 95% homologous to SEQ ID NO: 21. In some embodiments, the first affinity receiver comprises a peptide that is at least 99% homologous to SEQ ID NO: 21. In some embodiments, the first affinity receiver comprises a peptide having SEQ ID NO: 21.

In some embodiments, the extracellular vesicle further comprises a first messenger protein. In some embodiments, the first messenger protein comprises PD-L1. In some embodiments, PD-L1 is an extracellular domain of PD-L1. In some embodiments, the extracellular domain of PD-L1 is linked to the affinity tag. In some embodiments, the extracellular vesicle further comprises a second messenger protein. In some embodiments, the second messenger protein comprises CD200. In some embodiments, CD200 is an extracellular domain of CD200. In some embodiments, the extracellular domain of CD200 is linked to a second affinity tag.

In some embodiments, the extracellular vesicle further comprises a first messenger protein and a second messenger protein. In some embodiments, the first and second messenger proteins are immune checkpoint moieties. In some embodiments, the first messenger protein is PD-L1 and the second messenger protein is CD200. In some embodiments, the first messenger protein is an extracellular domain of PD-L1 and the second messenger protein is an extracellular domain of CD200. In some embodiments, the extracellular vesicle is for the treatment of ocular graft versus host disease.

In some embodiments, the first messenger protein is an immune checkpoint moiety and the second messenger protein is a growth factor. In some embodiments, the first messenger protein is PD-L1 and the second messenger protein is BDNF. In some embodiments, the first messenger protein comprises CD200. In some embodiments, the CD200 is an extracellular domain of CD200. In some embodiments, the second messenger protein comprises BDNF. In some embodiments, the BDNF is an extracellular domain of BDNF. In some embodiments, the second messenger protein comprises PD-L1. In some embodiments, the PD-L1 is an extracellular domain of PD-L1. In some embodiments, the extracellular vesicle is for the treatment of multiple sclerosis.

Disclosed herein, in some embodiments, is a complex, comprising an extracellular vesicle and an affinity tag bound to the first affinity receiver of the first recombinant protein, wherein the affinity tag is portion of a fusion protein comprising a first portion comprising the affinity tag and a second portion comprising a messenger protein.

In some embodiments, the messenger protein comprises an immune checkpoint moiety. In some embodiments, the messenger protein comprises an inhibitory immune checkpoint molecule. In some embodiments, the messenger protein comprises a stimulatory immune checkpoint molecule. In some embodiments, the messenger protein comprises an immune checkpoint moiety. In some embodiments, the messenger protein comprises a growth factor. In some embodiments, the growth factor comprises BDNF. In some embodiments, the growth factor comprises GDNF. In some embodiments, the messenger protein comprises PD-L1. In some embodiments, PD-L1 is an extracellular domain of PD-L1. In some embodiments, the messenger protein comprises CD200.

In some embodiments, the extracellular vesicle comprises a second messenger protein. In some embodiments, the second messenger protein comprises CD200. In some embodiments, the CD200 is an extracellular portion of CD200. In some embodiments, the second portion is an extracellular domain of an immune checkpoint moiety.

In some embodiments, the affinity tag comprises a peptide having an amino acid sequence that is configured to bind to a portion of the affinity receiver. In some embodiments, the affinity tag binds to an affinity receiver through a covalent bond. In some embodiments, the affinity tag binds to an affinity receiver through an iso-peptide bond. In some embodiments, the affinity tag binds to an affinity receiver through a di-sulfide bond.

An aspect of this instant disclosure is a method of delivering a messenger protein, comprising creating a fusion protein comprising the messenger protein and an affinity tag, adding the fusion protein to an extracellular vesicle, wherein the affinity tag binds to the first affinity receiver of the first recombinant protein.

Disclosed herein, in some embodiments, is a method of treating a disease in a patient, comprising administering to the patient a therapeutically effective amount of a pharmaceutical composition comprising a complex. In some embodiments, the disease is an autoimmune disease. In some embodiments, the disease is a neurological disease.

Disclosed herein, in some embodiments, is a method of treatment of ocular graft versus host disease, comprising administering a therapeutically effective amount of the extracellular vesicle. An aspect of this instant disclosure is a method of treatment of multiple sclerosis, comprising administering a therapeutically effective amount of the extracellular vesicle.

### BRIEF DESCRIPTION OF THE DRAWINGS

This patent application contains at least one drawing executed in color. Copies of this patent or patent application with color drawing(s) can be provided by the Office upon request and payment of the necessary fee.

FIG. 1 is a diagram illustrating an exosome loaded with passenger molecules using an affinity tag-receiver system.

FIG. 2 is a diagram showing 3 different messenger proteins on the surface of an exosome.

FIG. 3 shows the map of an embodiment of a recombinant protein having a transmembrane moiety and an affinity receiver (LAMP2_mNeonGreen).

FIG. 4 shows the map of an embodiment of a recombinant protein having a transmembrane moiety and an affinity receiver (LAMP2_Puro).

FIG. 5 shows the map of an embodiment of a recombinant protein having a transmembrane moiety and an affinity receiver (PTGFR_mNeonGreen).

FIG. 6 shows the map of an embodiment of a recombinant protein having a transmembrane moiety and an affinity receiver (PTGFR_Puro).

FIG. 7 shows the map of an embodiment of a recombinant protein having a transmembrane moiety and an affinity receiver (IGSF8_mNeonGreen).

FIG. 8 shows the map of an embodiment of a recombinant protein having a transmembrane moiety and an affinity receiver (IGSF8_Puro).

FIGS. 9A-9B show engineered exosomes suppress activated T cells and induce regulatory T cells (Tregs). FIG. 9A. PBMCs were activated with IL-2 (500 U/mL) and co-cultured with engineered and unmodified exosomes. Following a 6-day incubation, the resulting PBMC population was analyzed via flow cytometry for CD8+/CD25+ T cells. FIG. 9B. PBMCs were activated using anti-CD3+anti-CD28 Dynabeads and co-cultured with engineered and unmodified exosomes. After 3 days, the Treg population (CD4+CD25+FOXP3+) was analyzed via flow cytometry. Paired t-tests were used to determine p values (n=3; ***p<0.001).

FIG. 10 illustrates an exemplary affinity tag-receiver passenger loading system on an extracellular vesicle (e.g., exosome).

FIG. 11 depicts a schematic representation of ExoView imaging characterization using exosomes.

FIGS. 12A-12B show ExoView images confirming the presence of PD-L1 on engineered exosomes. FIG. 12B shows the presence of PD-L1 (labelled in green) on the surface of engineered exosomes. FIG. 12A shows unmodified exosomes (for use as a negative control) where PD-L1 is not present on the surface of the exosomes.

FIGS. 13A-13B show ExoView images confirming the presence of CD200 on engineered exosomes. FIG. 13B shows the presence of CD200 (labelled in green) on the surface of engineered exosomes. FIG. 13A shows unmodified exosomes where CD200 is not present on the surface of the exosomes.

FIGS. 14A-14B show ExoView images confirming the presence of PD-L1 and CD200 on engineered exosomes. FIG. 14B shows the presence of PD-L1 and CD200 (labeled in green and red respectively) on the surface of the engineered exosomes. Particles that appear as yellow are labeled with both green and red, representing a single particle that displays both PD-L1 and CD200. FIG. 14A shows unmodified exosomes where neither PD-L1 nor CD200 are present on the surface of the exosomes.

FIGS. 15A-15B show engineered exosomes activity on T cells as compared to unmodified exosomes. FIG. 15A shows flow cytometry readouts that display the percentage of activated T cells as defined by the presence of both CD8 and CD25 present in the analyzed cell population in the top right corner of each graph. FIG. 15B shows percentages of T cell activation. Significance was determined using the Student's t test (p<0.0001).

FIG. 16A illustrates a schematic representation of an assay performed to evaluate the biologic activity of engineered exosomes on inflammatory macrophage inhibition using cytokine analysis. FIG. 16B depicts levels of IL-1β that were measured using unmodified exosomes as compared to engineered exosomes. Significance was determined using the Student's t test.

FIGS. 17A-17B show the adjustment of engineered exosome biological activity by tuning ligand density. FIG. 17A shows flow cytometry readouts that display the percentage of activated T cells as defined by the presence of both CD8 and CD25 present in the analyzed cell population in the top right corner of each graph. FIG. 17B shows the percentage of activated T cells. Significance was determined using the Student's t test. 1× Ligand Density group were loaded with 10 molecules/exosome. 2× Ligand Density group were loaded with 25 molecules/exosome.

FIGS. 18A-18B show that biological activity of exosomes with multiple ligands is comparable to exosomes displaying a single ligand. FIG. 18A shows flow cytometry readouts that display the percentage of activated T cells as defined by the presence of both CD8 and CD25 present in the analyzed cell population in the top right corner of each graph.

FIG. 18B shows the percentage of activated T cells. Significance was determined using the Student's t test. 1 Ligand Exosomes were fully loaded with PD-L1, and Traditional Exosomes were engineered using traditional engineering methods. Combination Exosomes express both PD-L1 and CD200.

FIG. 19 shows four exemplary constructs that may be used as a CRISPR template for LAMP2B.

FIG. 20 shows four exemplary constructs that may be used as a CRISPR template for PTGFR.

FIG. 21 shows four exemplary constructs that may be used as a CRISPR template for IGSF8.

The novel features of the disclosure are set forth with particularity in the appended claims. A better understanding of the features and advantages of the present disclosure can be obtained by reference to the following detailed description that sets forth illustrative embodiments.

### DETAILED DESCRIPTION

Described herein are compositions, methods, and systems for loading proteins, peptides, or other molecules (e.g., passenger proteins (e.g., immune checkpoint moieties)) to the surface of an extracellular vesicle (e.g., exosome). In some embodiments, at least a portion of an exosome specific protein (e.g., Cd63, cd81, and Lamp-2) serves as an anchor protein and is fused with a peptide that can bind to an affinity tag. The protein, peptide, or molecule to be loaded (e.g., “passenger” (e.g., passenger protein, passenger peptide, or passenger molecule) is linked (e.g., fused) with a peptide affinity tag that can bind to the affinity receiver on the anchor protein. Thus, when a passenger protein having the affinity tag is mixed with an exosome carrying the anchor protein having the affinity receiver, the affinity tag binds the affinity receiver, bringing the passenger protein to the surface of the exosome.

The loading approach provided herein utilizes an affinity receiver-affinity tag system and provides superior methods for loading protein(s) onto extracellular vesicles as compared to traditional methods, where some passenger proteins cannot be directly loaded onto exosomes, for example, secreted growth factors and large proteins.

Furthermore, the extracellular vesicles disclosed herein may be useful for adjusting an amount of passenger protein(s) (e.g., PD-L1 and CD200) on the surface of the exosome. It is contemplated that the density of passenger proteins on the surface of the exosome can be adjusted or finetuned to target a disease (e.g., GVHD). The adjustment may be achieved by changing the amount of passenger proteins added to the exosome, or by changing the density of anchor proteins on the exosome, or both. It is also contemplated that the binding affinity of the passenger proteins to the exosome may be adjusted by using different peptide ligands that have higher or lower affinity to the affinity receiver. This tunable platform is uniquely suitable for adjustable, personalized, and precise delivery of therapeutic molecules to specific destinations in order to treat certain diseases, including GVHD, autoimmune diseases (e.g., multiple sclerosis), neurological diseases (e.g., Huntington's disease, Alzheimer's disease), and cancer.

In particular, the adjustability of the amount or ration of passenger protein on the extracellular vesicle may be useful for modifying an amount or activity of one or more biologic targets. For example, an amount of an immune checkpoint may be adjusted depending on the desired effect. Furthermore, the amount or ratio of a first immune checkpoint moiety may be adjusted in relation to an amount or ratio of a second immune checkpoint moiety. As a further example, the amount of PD-L1 displayed on the extracellular vesicle may be adjusted to adjust for a desired effect on T cell activity, whereas the amount of CD200 may be adjusted to adjust for an effect on macrophage activity. In fact, the extracellular vesicle comprising an affinity receiver presented herein may be useful for controlling the amount or density of the passenger protein on the surface of the extracellular vesicle.

As used herein, “platform” and “system” are used interchangeably.

In addition to allowing for the capability of quickly adjusting the amount or ratio of immune checkpoint moieties, the engineered extracellular vesicle provided herein may provide for an opportunity to quickly adjust the type of immune checkpoint moieties using the same cell line that produces the extracellular vesicle. For example, since one cell line will be capable of producing extracellular vesicles that exhibit one or more types of affinity receiver, one would be capable of quickly attaching different passenger proteins and affinity tags to the extracellular vesicle. For example, a cell line producing extracellular vesicles expressing an affinity tag may be used to produce extracellular vesicles exhibiting a first immune checkpoint moiety and a second immune checkpoint moiety, while also being used to produce extracellular vesicles exhibiting a third immune checkpoint moiety and a fourth immune checkpoint moiety. This quick tunability may be achieved by use of the e extracellular vesicle comprising an affinity receiver disclosed herein. In fact, the linking of an affinity tag (e.g., attached to a passenger protein) to an affinity receiver has been completed in one hour. This allows for faster modification of the extracellular vesicle comprising an affinity receiver, which in turn may lead to faster research and faster creation of the engineered extracellular vesicle for use in treating certain diseases (e.g., GVHD).

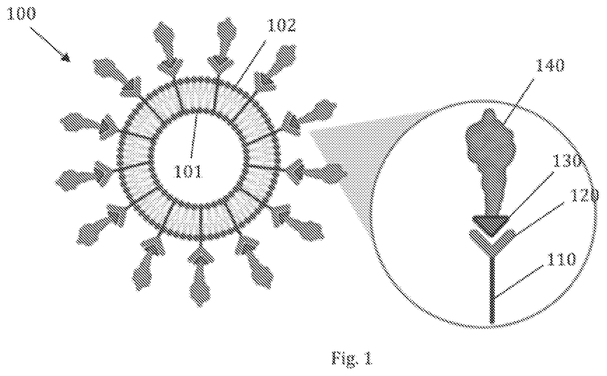

In FIG. 1, an extracellular vesicle 100 has a membrane made of two layer of lipid molecules, an inner layer 101 and an outer layer 102. A recombinant protein has a transmembrane moiety 110 and an affinity receiver 120. The affinity receiver 120 binds to an affinity tag 130. The affinity tag 130 is part of a fusion protein that comprises a messenger protein 140.

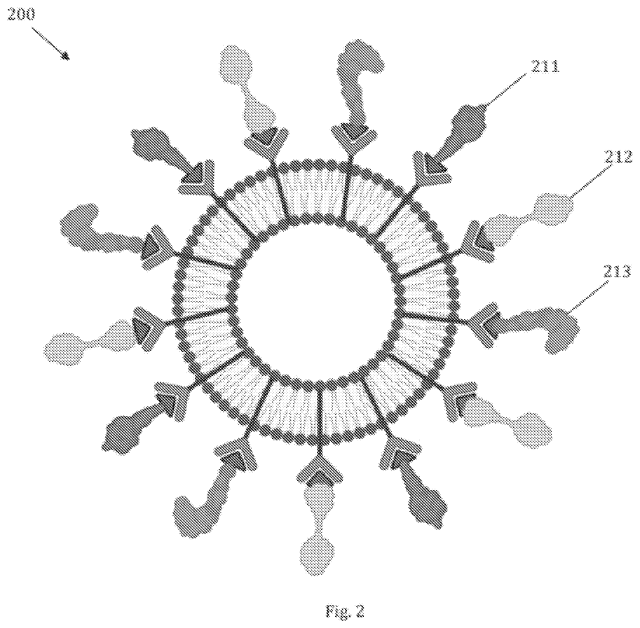

In FIG. 2, three different messenger proteins (211-213) are on the surface of an exosome 200 through the affinity tag-receiver binding system.

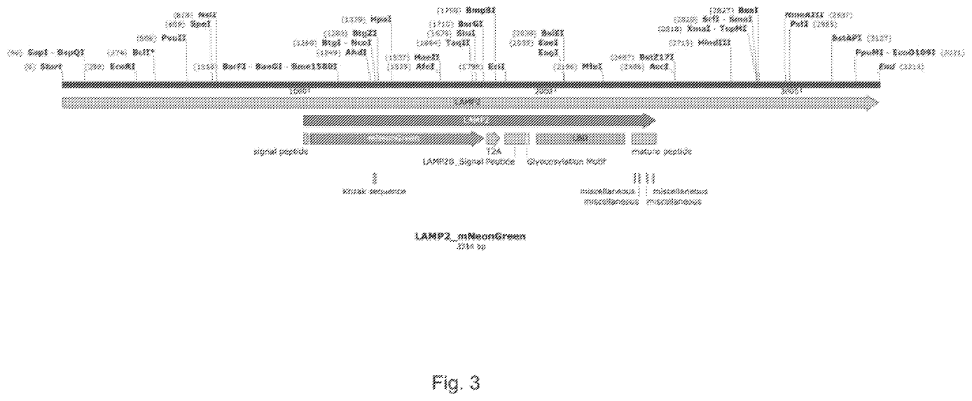

In FIG. 3, the affinity receiver is labeled “LBD”, and the transmembrane moiety is labeled “LAMP2”. The affinity receiver has the amino acid sequence

(SEQ ID NO: 20)
MVTTLSGLSGEQGPSGDMTTEEDSATHIKFSKRDEDGRELAGATM
ELRDSSGKTISTWISDGHVKDFYLYPGKYTFVETAAPDGYEVATP
IEFTVNEDGQVTVDGEATEGDAHTGSSGS.

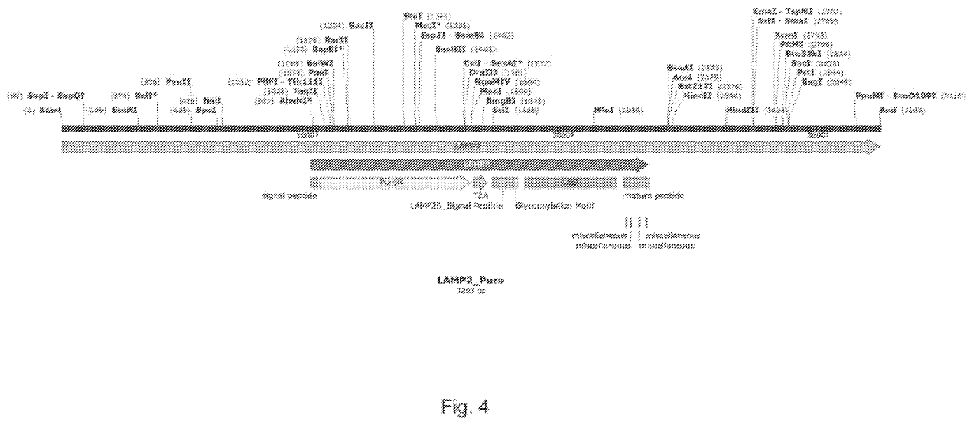

In FIG. 4, the affinity receiver is labeled “LBD”, and the transmembrane moiety is labeled “LAMP2”.

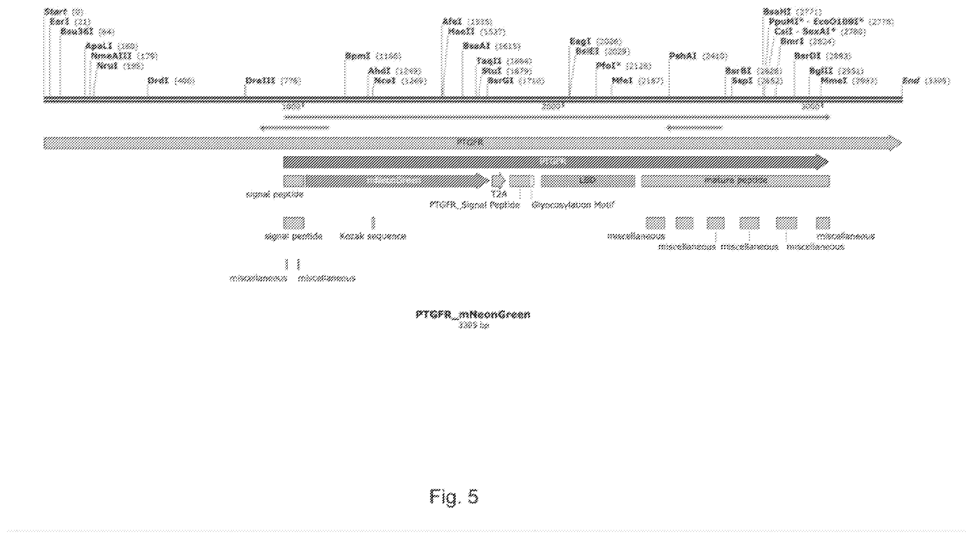

In FIG. 5, the affinity receiver is labeled “LBD”, and the transmembrane moiety is labeled “PTGFR”.

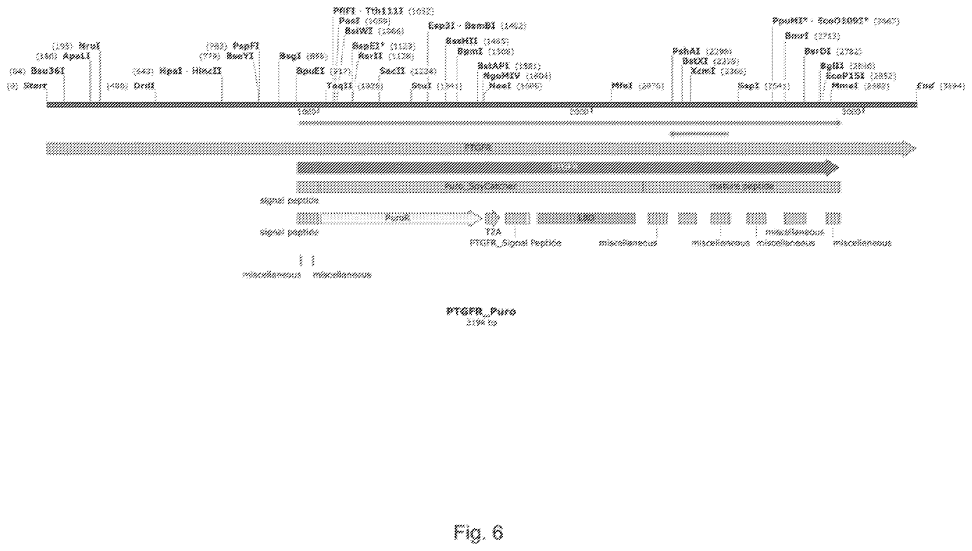

In FIG. 6, the affinity receiver is labeled “LBD”, and the transmembrane moiety is labeled “PTGFR”.

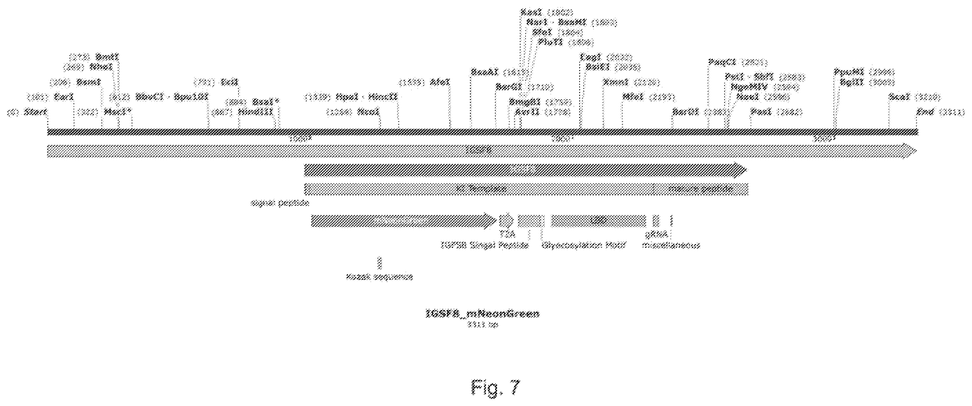

In FIG. 7, the affinity receiver is labeled “LBD”, and the transmembrane moiety is labeled “IGSF8”.

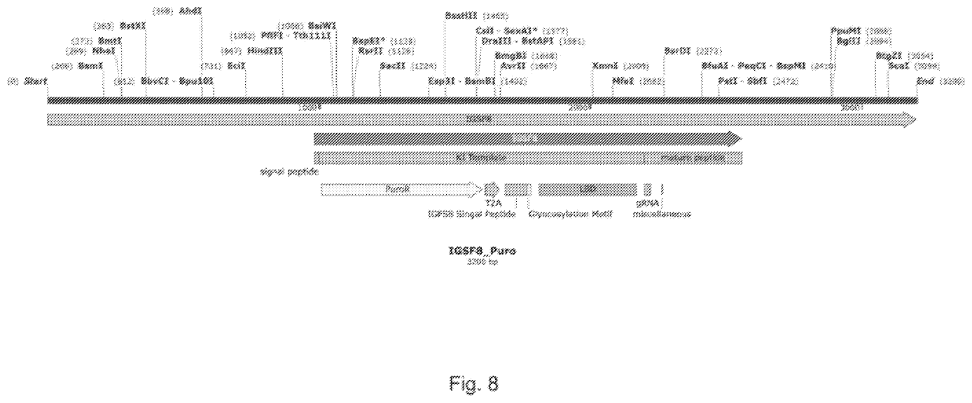

In FIG. 8, the affinity receiver is labeled “LBD”, and the transmembrane moiety is labeled “IGSF8”.

### Transmembrane Moiety

Described herein, in some embodiments, are compositions comprising an engineered extracellular vesicle comprising a recombinant protein having at least one transmembrane moiety. In some embodiments, the transmembrane moiety comprises a full-length protein or a variation thereof or a fragment thereof. In some embodiments, the transmembrane moiety is endogenous to the cell that is generating the engineered extracellular vesicles. In some embodiments, the transmembrane moiety is selected from a group consisting of: 14-3-3 protein zeta/delta, 14-3-3 protein epsilon, 78 kDa glucose-regulated protein, acetylcholinesterase/AChE-S, AChE-E, actin, cytoplasmic 1 (ACTA), ADAM10, alkaline phosphatase, alpha-enolase, alpha-synuclein, aminopeptidase N, amyloid beta A4/APP, annexin 5A, annexin A2, AP-1, ATF3, ATP citrate lyase, ATPase, beta actin (ACTB), beta-amyloid 42, caveolin-1, CD10, CD11a, CD11b, CD11c, CD14, CD142, CD146, CD163, CD24, CD26/DPP4, CD29/ITGB1, CD3, CD37, CD41, CD42a, CD44, CD45, CD47, CD49, CD49d, CD53, CD63, CD64, CD69, CD73 CD81, CD82, CD9, CD90, claudin, claudin-1 cofilin-1, complement-binding proteins CD55 and CD59, cytosolic heat shock protein 90 alpha, cytosolic heat shock protein 90 beta, EBV LMP1, EBV LMP2A, EF-1alpha-1, EF2, EFGR EGFR VIII, emmprin/CD147, enolase 1 alpha (ENO1), EPCAM, ERBB2, tetraspanins (CD9, CD63 and CD81), fatty acid synthase, fetuin-A, flotillin-1, flotillin-2, fructose-bisphosphate aldolase A, glyceraldehyde-3-phosphate dehydrogenase (GAPDH), glycophorin A, GPC1, GPI-anchored 5′nucleotidase, GTPase, heat shock protein 8 (HSPA8), heat shock proteins (HSP70 and HSP90), heparan sulfate proteoglycans, heparinase, heterotrimeric G proteins, HIV Gag, HIV Nef, HLA-DRA, HLA-G, HSV gB, HTLV-1 Tax, huntingtin, ICAM1, integrins, lactadherin, LAMP1/2, LAMP2A, LAMP2B, LAMP2C, leucine-rich receptor kinase 2, L-lactate dehydrogenase A chain, lysosome-associated membrane glycoprotein 1, lysosome-associated membrane glycoprotein 2, MHC class I, MHC class II, MUC1, multidrug resistance-associated protein, muscle pyruvate kinase (PKM2), N-cadherin, NKCC2, PDCD6IP/Alix, PECAM1, phosphoglycerate kinase, placental prion proteins, prostate-specific antigen (PSA), pyruvate kinase (PKM), Rab-14, Rab-5a, Rab-5b, Rab-5c, Rab-7, Rap 1B, resistin, sonic hedgehog (SHH), surviving, syndecan-1, syndecan-4, syntenin-1, transferrin receptor (TFR2), TSG101, TSPAN8, tumor-associated glycoprotein tetraspanin-8, tyrosine 3 monooxygenase/tryptophan 5-monooxygenase activation protein, TYRP-2, or vacuolar-sorting-associated protein 35 (VPS35).

In some embodiments, the transmembrane moiety comprises LAMP2. In some embodiments, the transmembrane moiety comprises LAMP-like domain 1 of LAMP2. In some embodiments, the transmembrane moiety comprises LAMP2B. In some embodiments, the transmembrane moiety comprises a polypeptide sequence that is at least 50%, 55%, 60%, 65%, 70%, 75%, 80%, 85%, 90%, 95%, or 99% identical to SEQ ID NO: 1 (Table 1). In some embodiments, the transmembrane moiety is encoded from a polynucleotide sequence comprising homology arms. In some instances, the immune checkpoint moiety described herein is inserted into the transmembrane moiety by homologous recombination as induced by the homology arms of the transmembrane moiety. In some embodiments, the transmembrane moiety comprises an N-terminus homology arm or a C-terminus homology arm. In some embodiments, the N-terminus homology of the transmembrane moiety is encoded from a polynucleotide sequence that is at least 50%, 55%, 60%, 65%, 70%, 75%, 80%, 85%, 90%, 95%, or 99% identical to SEQ ID NO: 2 (Table 1). In some embodiments, the C-terminus homology of the transmembrane moiety is encoded from a polynucleotide sequence that is at least 50%, 55%, 60%, 65%, 70%, 75%, 80%, 85%, 90%, 95%, or 99% identical to SEQ ID NO: 3 (Table 1).

In some embodiments, the transmembrane moiety comprises the signal peptide or a fragment thereof of LAMP2. In some embodiments, the transmembrane moiety comprises the signal peptide or a fragment thereof of LAMP-like domain 1 of LAMP2. In some embodiments, the transmembrane moiety comprises the signal peptide or a fragment thereof of LAMP2B. In some embodiments, the transmembrane moiety comprises a sequence that is at least 50%, 55%, 60%, 65%, 70%, 75%, 80%, 85%, 90%, 95%, or 99% identical to any one of SEQ ID NOS: 31-32 (Table 1).

In some embodiments, the transmembrane moiety comprises a prostaglandin receptor or a fragment thereof. In some embodiments, the transmembrane moiety comprises the prostaglandin selected from a group consisting of prostaglandin DP1 receptor, prostaglandin DP2 receptor, prostaglandin EP1 receptor, prostaglandin EP2 receptor, prostaglandin EP3 receptor, prostaglandin EP4 receptor, prostaglandin F2a receptor, prostacyclin 12 receptor, and thromboxane A2 receptor, or a fragment thereof. In some embodiments, the transmembrane moiety comprises prostaglandin F receptor or a fragment thereof. In some embodiments, the transmembrane moiety comprises PTGFR or a fragment thereof. In some embodiments, the transmembrane moiety comprises the signal peptide or a fragment thereof of the prostaglandin receptor. In some embodiments, the transmembrane moiety is the signal peptide or a fragment thereof of the prostaglandin receptor selected from a group consisting of prostaglandin DP1 receptor, prostaglandin DP2 receptor, prostaglandin EP1 receptor, prostaglandin EP2 receptor, prostaglandin EP3 receptor, prostaglandin EP4 receptor, prostaglandin F2a receptor, prostacyclin 12 receptor, and thromboxane A2 receptor. In some embodiments, the transmembrane moiety comprises the signal peptide or a fragment thereof of the prostaglandin F receptor. In some embodiments, the transmembrane moiety comprises the signal peptide of a fragment thereof of PTGFR. In some embodiments, the transmembrane moiety comprises a sequence that is at least 50%, 55%, 60%, 65%, 70%, 75%, 80%, 85%, 90%, 95%, or 99% identical to any one of SEQ ID NOS: 33-34 (Table 1).

In some embodiments, the transmembrane moiety comprises immunoglobulin superfamily member (IGSF). In some embodiments, the transmembrane is selected from a group consisting of IGSF1, IGSF2, IGSF3, IGSF4, IGSF5, IGSF6, IGSF7, IGSF8, IGSF9, IGSF10, IGSF12, and IGSF13. In some embodiments, the transmembrane moiety comprises IGSF8. In some embodiments, the transmembrane moiety comprises the signal peptide or a fragment thereof of immunoglobulin superfamily member (IGSF). In some embodiments, the transmembrane is the signal peptide or a fragment thereof of the immunoglobulin superfamily member (IGSF) selected from a group consisting of IGSF1, IGSF2, IGSF3, IGSF4, IGSF5, IGSF6, IGSF7, IGSF8, IGSF9, IGSF10, IGSF12, and IGSF13. In some embodiments, the transmembrane moiety comprises the signal peptide or a fragment thereof of IGSF8. In some embodiments, the transmembrane moiety comprises a sequence that is at least 50%, 55%, 60%, 65%, 70%, 75%, 80%, 85%, 90%, 95%, or 99% identical to any one of SEQ ID NOS: 35-36 (Table 1).

TABLE 1
Exemplary polynucleotide sequence of
transmembrane moiety

| LAMP2B polypeptide sequence, accession | |
|---|---|
| number P13473-2 | |
| SEQ ID NO: 1 | MVCFRLFPVPGSGLVLVCLVLGAVRSYALELNLT |
| | DSENATCLYAKWQMNFTVRYETTNKTYKTVTISD |
| | HGTVTYNGSICGDDQNGPKIAVQFGPGFSWIANF |
| | TKAASTYSIDSVSFSYNTGDNTTFPDAEDKGILT |
| | VDELLAIRIPLNDLFRCNSLSTLEKNDVVQHYWD |
| | VLVQAFVQNGTVSTNEFLCDKDKTSTVAPTIHTT |
| | VPSPTTTPTPKEKPEAGTYSVNNGNDTCLLATMG |
| | LQLNITQDKVASVININPNTTHSTGSCRSHTALL |
| | RLNSSTIKYLDFVFAVKNENRFYLKEVNISMYLV |
| | NGSVFSIANNNLSYWDAPLGSSYMCNKEQTVSVS |
| | GAFQINTFDLRVQPFNVTQGKYSTAQDCSADDDN |
| | FLVPIAVGAALAGVLILVLLAYFIGLKHHHAGYE |
| | QF |

LAMP2B N-terminus homology arm

| | |
|---|---|
| SEQ ID NO: 2 | cttaaatggttgcccaatatttatgctgctggca |
| | tgtttgctgggatattttttctcctctgaattag |
| | tattttgagtccttgggctcttcagtttaataga |
| | taatgtttctatgtttaagtgtagaagatccacc |
| | atgttagctgaaaagttccaaggcttttctgtct |
| | ctgtaacatttctctgcctaaagaggtagcaggt |
| | tgaaaatttaggaaataactttgcgtcaaaaaat |
| | tcatcagcctttctaatcaattcgattttaaagt |
| | gaaacagtgatgaactttgcatttatcagaattc |
| | atgtttttctcatggatttaacagccctttgcaa |
| | ctctgcttccttggttacatgatagaaagagtat |
| | ctgatcacatcagaataaaatcgtttgggaagac |
| | atccgtgtgtcatagattatgtagcttgcaacag |
| | gatttaaagcaaaaagatggtagaatatcggatc |
| | tatcattatttttgcagtgaatgtttcaccagct |
| | ggtttgagagaagttttacacccctaccaggctt |
| | cctcttacctcatttaccgcaaaaaccacaagtc |
| | ctaacaggcaaactgtaattgcctaaaacaaaac |
| | tagtaaggtataaatgcatgtggaaatgattatt |
| | tgaaggcaaaaggcaaataaatgtcaccagtctg |
| | agccatgacctaagcatcctaggtctagtaacca |
| | ttcttttggtaagggataatgccaacttttttat |
| | caataggattctgttaaaaatacaaagaaaaaca |
| | gtcagcaggatactgtatctatacacatacatat |
| | atgtatatacacatatatgtatatatatgtatgt |
| | gtatatatagatataaaatacttttgtttcattt |
| | tagagctggttgaacttcagtggtgggtagagct |
| | tgtttttaatttatataatttttgtagtacaatt |
| | ttaaaatattatttttttaaatgaatccagg |

LAMP2B C-terminus homology arm

| | |
|---|---|
| SEQ ID NO: 3 | gaacttaatttgacagattcagaaaatgccactt |
| | gcctttatgcaaaatggcagatgaatttcacagt |
| | acgctatgaaactacaaataaaacttatgtaagt |
| | atatattttaatttttctttgagttttatagtag |
| | gaatttaattgactttatcctttagatacacgta |
| | tacaaatgaaattccacgttgactgacttaatgc |
| | tgaaactctgggattttttttaagcatacacact |
| | cagaagttattctgtttagtgttagaatgaatat |
| | ttcttctgattcattggttcttgggagtttttgt |
| | aggacttgattaagggcaaatgatctgaagacga |
| | ctatatggtgttcaggtctaagtatttctgtaaa |
| | gtgtttaaaatgtactagcaccaaagcttgccct |
| | tttaaaaagcagcttcccatctgggcatggtggc |
| | tcatgcctgtgatcccagcattttggaaggccaa |
| | agcaggaggattgcttgaacccagcccgggcaac |
| | atggtgagacctcatctctacaaaaaaactttaa |
| | aaattagccaggcatggtggcacacacatgtacc |
| | tccagccactcgctgggccgaggctggaggattg |
| | cttgagctcaggaggttgagtctgcagtgagcca |
| | tgattgtgctactgcactccagcctgggcaacag |
| | agcgagactctatctcaaaaaaaaaaaaaatcgc |
| | ttacttaggttggtacaaaagtaattgccattaa |
| | aggaacaaaccacaattacttttgcaccaaccta |
| | agtaagcgatttttttttttggcacaaactgcaa |
| | ttacttttgcaccaacctaacactaaaatctccc |
| | cctttttgacatctcctcttaattcctttgcctt |
| | cttattttctttttgttaaggtcctcactactta |
| | caacctaattttttaaaaaatcagtttaagaaca |
| | gcagctcaagccagttttcctttttttttagaca |
| | tacacgaa |

LAMP2B Signal Peptide (Gene ID: 3920)

| | |
|---|---|
| SEQ ID NO: | atggtgtgcttccgcctcttcccggttccgggct |
| 31 | cagggctcgttctggtctgcctagtcctgggagc |
| | tgtgcggtcttatgca |
| SEQ ID NO: | MVCFRLFPVPGSGLVLVCLVLGAVRSYA |
| 32 | |

PTGFR Signal Peptide (Gene ID: 5737)

| | |
|---|---|
| SEQ ID NO: | atgtccatgaacaattccaaacagctagtgtctc |
| 33 | ctgcagctgcgcttctttcaaacacaacctgcca |
| | gacggaa |
| SEQ ID NO: | MSMNNSKQLVSPAAALLSNTTCQTE |
| 34 | |

IGSF8 Signal Peptide (Gene ID: 93185)

| | |
|---|---|
| SEQ ID NO: | atgggcgccctcaggcccacgctgctgccgcctt |
| 35 | cgctgccgctgctgctgctgctaatgctaggaat |
| | gggatgctgggcc |
| SEQ ID NO: | MGALRPTLLPPSLPLLLLLMLGMGCWA |
| 36 | |

In some embodiments, the fusion protein or polypeptide comprises at least one transmembrane moiety. In some embodiments, the transmembrane moiety comprises a full-length protein or a variation thereof or a fragment thereof. In some embodiments, the transmembrane moiety is endogenous to the cell that is generating the engineered extracellular vesicles. In some embodiments, the immune checkpoint moiety is heterologous to the cell that is generating the engineered extracellular vesicles. In some embodiments, the transmembrane moiety is selected from a group consisting of: 14-3-3 protein zeta/delta, 14-3-3 protein epsilon, 78 kDa glucose-regulated protein, acetylcholinesterase/AChE-S, AChE-E, actin, cytoplasmic 1 (ACTA), ADAM10, alkaline phosphatase, alpha-enolase, alpha-synuclein, aminopeptidase N, amyloid beta A4/APP, annexin 5A, annexin A2, AP-1, ATF3, ATP citrate lyase, ATPase, beta actin (ACTB), beta-amyloid 42, caveolin-1, CD10, CD11a, CD11b, CD11c, CD14, CD142, CD146, CD163, CD24, CD26/DPP4, CD29/ITGB1, CD3, CD37, CD41, CD42a, CD44, CD45, CD47, CD49, CD49d, CD53, CD63, CD64, CD69, CD73 CD81, CD82, CD9, CD90, claudin, claudin-1 cofilin-1, complement-binding proteins CD55 and CD59, cytosolic heat shock protein 90 alpha, cytosolic heat shock protein 90 beta, EBV LMP1, EBV LMP2A, EF-1alpha-1, EF2, EFGR EGFR VIII, emmprin/CD147, enolase 1 alpha (ENO1), EPCAM, ERBB2, tetraspanins (CD9, CD63 and CD81), fatty acid synthase, fetuin-A, flotillin-1, flotillin-2, fructose-bisphosphate aldolase A, glyceraldehyde-3-phosphate dehydrogenase (GAPDH), glycophorin A, GPC1, GPI-anchored 5′nucleotidase, GTPase, heat shock protein 8 (HSPA8), heat shock proteins (HSP70 and HSP90), heparan sulfate proteoglycans, heparinase, heterotrimeric G proteins, HIV Gag, HIV Nef, HLA-DRA, HLA-G, HSV gB, HTLV-1 Tax, huntingtin, ICAM1, integrins, lactadherin, LAMP1/2, LAMP2A, LAMP2B, LAMP2C, leucine-rich receptor kinase 2, L-lactate dehydrogenase A chain, lysosome-associated membrane glycoprotein 1, lysosome-associated membrane glycoprotein 2, MHC class I, MHC class II, MUC1, multidrug resistance-associated protein, muscle pyruvate kinase (PKM2), N-cadherin, NKCC2, PDCD6IP/Alix, PECAM1, phosphoglycerate kinase, placental prion proteins, prostate-specific antigen (PSA), pyruvate kinase (PKM), Rab-14, Rab-5a, Rab-5b, Rab-5c, Rab-7, Rap 1B, resistin, sonic hedgehog (SHH), surviving, syndecan-1, syndecan-4, syntenin-1, transferrin receptor (TFR2), TSG101, TSPAN8, tumor-associated glycoprotein tetraspanin-8, tyrosine 3 monooxygenase/tryptophan 5-monooxygenase activation protein, TYRP-2, or vacuolar-sorting-associated protein 35 (VPS35)

In some embodiments, the transmembrane moiety is encoded by a sequence that is at least 50%, 55%, 60%, 65%, 70%, 75%, 80%, 85%, 90%, 95%, or 99% identical to any one of SEQ ID NOS: 31-32 (Table 1). In some embodiments, the transmembrane moiety is encoded by a sequence that is at least 50%, 55%, 60%, 65%, 70%, 75%, 80%, 85%, 90%, 95%, or 99% identical to SEQ ID NOS: 33-34 (Table 1). In some embodiments, the transmembrane moiety is encoded by a sequence that is at least 50%, 55%, 60%, 65%, 70%, 75%, 80%, 85%, 90%, 95%, or 99% identical to any one of SEQ ID NOS: 35-36.

In some embodiments, the transmembrane moiety is LAMP2 and encoded from polynucleotide or a variation thereof or a fragment thereof that is at least 50%, 55%, 60%, 65%, 70%, 75%, 80%, 85%, 90%, 95%, or 99% identical to any one of SEQ ID NOS: 31-32 (Table 1). In some embodiments, the transmembrane moiety is PTGFR and encoded from polynucleotide or a variation thereof or a fragment thereof that is at least 50%, 55%, 60%, 65%, 70%, 75%, 80%, 85%, 90%, 95%, or 99% identical to any one of SEQ ID NOS: 33-34 (Table 1). In some embodiments, the transmembrane moiety is IGSF8 and encoded from polynucleotide or a variation thereof or a fragment thereof that is at least 50%, 55%, 60%, 65%, 70%, 75%, 80%, 85%, 90%, 95%, or 99% identical to any one of SEQ ID NOS: 35-36 (Table 1).

### Immune Checkpoint Moiety

Described herein, in some cases, are engineered extracellular vesicles comprising at least one immune checkpoint moiety. In some embodiments, the immune checkpoint moiety is endogenous to the cell that is generating the engineered extracellular vesicles. In some embodiments, the immune checkpoint moiety is heterologous to the cell that is generating the engineered extracellular vesicles. In some embodiments, the immune checkpoint moiety comprises therapeutic properties for treating diseases or disorders. In some embodiments, the immune checkpoint moiety comprises therapeutic properties for treating an inflammatory or autoimmune disease or disorder described herein. In some embodiments, the immune checkpoint moiety targets and modulates activities of immune cells. In some embodiments, the immune cells comprise T cell, including cytotoxic T cell, Natural Killer T cell, Regulatory T cell, and T helper cells. In some embodiments, the immune cells comprises CD8+ cells. In some embodiments, the immune cells comprise CD25+ cells. In some embodiments, the immune cells comprise CD4+ cells. In some embodiments, the immune cells comprise CD8+CD25+ cells. In some cases, the immune cell comprise cell that expresses CD4. In some cases, the immune cell comprises cell that expresses CD4 and CD 25 (CD4+CD25+). In some cases, the immune cell comprise cell that expresses FOXP3. In some cases, the immune cell comprise cell that expresses CD4, CD25, and FOXP3 (CD4+CD25+FOXP3+).

In some embodiments, the engineered extracellular vesicle comprises a first immune checkpoint moiety and a second immune checkpoint moiety, wherein the first immune checkpoint moiety is an extracellular domain of the first immune checkpoint moiety (e.g., CD200) and the second immune checkpoint moiety is an extracellular domain of the second immune checkpoint moiety.

In some embodiments, the immune checkpoint moiety can comprise any one of VISTA, PD-L1, CTLA-4, PD-L2, B7-1 (CD80), B7-2 (CD86), B7-H3 (CD276), B7-H2, B7-H3, B7-H4 (VTCN1), IDO, KIR, LAG3, A2AR, HVEM (CD270, TNFRSF14), Galectin 9, Galectin3, CEACAM1 (CD66a), OX-2 (CD200), PVR (CD155), PVRL2 (Nectin-2, CD112), FGL-1, PECAM-1, TSG-6, CD47, Stabilin-1 (Clever-1), Neuropilin 1, Neuropilin 2, CD158 (family), IGSF2 (CD101), CD155, GITRL, CD137L, OX40L, LIGHT, CD70, PD-1, RGMB, CTLA-4 (CD152), BTLA, CD160, Tim-3, CD200R, TIGIT, CD112R (PVRIG), LAG-3 (CD223), PECAM-1, CD44, IGSF11 (VSIG-3), or SIRP alpha (CD172a).

In some embodiments, the immune checkpoint moiety is PD-L1. In some embodiments, the immune checkpoint moiety (e.g., a first immune checkpoint moiety) is an extracellular domain of PD-L1. In some embodiments, the immune checkpoint moiety (e.g., a second immune checkpoint moiety) is CD200. In some embodiments, the immune checkpoint moiety is an extracellular domain of CD200. In some embodiments, the immune checkpoint moiety is a plurality of immune checkpoint moieties. In some embodiments, the plurality of immune checkpoint moieties is PD-L1 and CD200. In some embodiments, the plurality of immune checkpoint moieties is the extracellular domain of PD-L1 and the extracellular domain of CD200.

In some embodiments, the engineered extracellular vesicle comprises an extracellular domain of a first immune checkpoint moiety (e.g., CD200) and a second extracellular domain of a second immune checkpoint moiety (e.g., PD-L1). In some embodiments, the engineered extracellular vesicle comprises a first immune checkpoint moiety that is an extracellular domain of CD200 and a second immune checkpoint moiety (e.g., an extracellular domain of the second immune checkpoint moiety).

In some embodiments, the immune checkpoint moiety comprises a polynucleotide sequence encoding a polypeptide or a variation thereof or a fragment thereof. In some embodiments, the immune checkpoint moiety comprises a polypeptide or a variation thereof or a fragment thereof. In some embodiments, the immune checkpoint moiety is covalently connected to the transmembrane moiety. In some embodiments, the immune checkpoint moiety is encoded by a polynucleotide sequence encoding VISTA, PD-L1, IGSF11 (VSIG-3), or CLLA-4.

In some embodiments, the immune checkpoint moiety comprises a sequence that is at least 10%, 20%, 30%, 40%, 50%, 60%, 70%, 75%, 80%, 85%, 90%, 95%, 99% or 100% homologous to SEQ ID NO: 37 (Table 2). In some embodiments, the immune checkpoint moiety comprises a sequence that is at least 10%, 20%, 30%, 40%, 50%, 60%, 70%, 75%, 80%, 85%, 90%, 95%, 99% or 100% homologous to the SEQ ID NO: 38 (Table 2). In some embodiments, the immune checkpoint moiety comprises a sequence that is at least 10%, 20%, 30%, 40%, 50%, 60%, 70%, 75%, 80%, 85%, 90%, 95%, 99% or 100% homologous to the SEQ ID NO: 39 (Table 2). In some embodiments, the immune checkpoint moiety comprises a sequence that is at least 10%, 20%, 30%, 40%, 50%, 60%, 70%, 75%, 80%, 85%, 90%, 95%, 99% or 100% homologous to the SEQ ID NO: 40 (Table 2). In some embodiments, the immune checkpoint moiety comprises a sequence that is at least 10%, 20%, 30%, 40%, 50%, 60%, 70%, 75%, 80%, 85%, 90%, 95%, 99% or 100% homologous to SEQ ID NO: 41 (Table 2). In some embodiments, the immune checkpoint moiety comprises a sequence that is at least 10%, 20%, 30%, 40%, 50%, 60%, 70%, 75%, 80%, 85%, 90%, 95%, 99% or 100% homologous to the SEQ ID NO: 42 (Table 2). In some embodiments, the immune checkpoint moiety comprises a sequence that is at least 10%, 20%, 30%, 40%, 50%, 60%, 70%, 75%, 80%, 85%, 90%, 95%, 99% or 100% homologous to the SEQ ID NO: 43 (Table 2). In some embodiments, the immune checkpoint moiety comprises a sequence that is at least 10%, 20%, 30%, 40%, 50%, 60%, 70%, 75%, 80%, 85%, 90%, 95%, 99% or 100% homologous to the SEQ ID NO: 44 (Table 2).

In some embodiments, the immune checkpoint moiety is encoded by a polynucleotide sequence that is at least 50%, 55%, 60%, 65%, 70%, 75%, 80%, 85%, 90%, 95%, or 99% identical to any one of SEQ ID NOs: 37-44 (Table 2).

TABLE 2
Exemplary polynucleotide sequence of immune checkpoint moiety

| | |
|---|---|
| | VISTA, Accession Number Q9H7M9 |
| SEQ ID NO: 37 | MGVPTALEAGSWRWGSLLFALFLAASLGPVAAFKVATPYSLYVCP |
| | EGQNVTLTCRLLGPVDKGHDVTFYKTWYRSSRGEVQTCSERRPIRN |
| | LTFQDLHLHHGGHQAANTSHDLAQRHGLESASDHHGNFSITMRNL |
| | TLLDSGLYCCLVVEIRHHHSEHRVHGAMELQVQTGKDAPSNCVVY |
| | PSSSQDSENITAAALATGACIVGILCLPLILLLVYKQRQAASNRRAQE |
| | LVRMDSNIQGIENPGFEASPPAQGIPEAKVRHPLSYVAQRQPSESGR |
| | HLLSEPSTPLSPPGPGDVFFPSLDPVPDSPNFEVI |
| SEQ ID NO: 38 | ttcaaggtcgccacgccgtattccctgtatgtctgtcccgaggggcagaacgtcaccctcacctgcaggctc |
| | ttgggccctgtggacaaagggcacgatgtgaccttctacaagacgtggtaccgcagctcgaggggcgagg |
| | tgcagacctgctcagagcgccggcccatccgcaacctcacgttccaggaccttcacctgcaccatggagg |
| | ccaccaggctgccaacaccagccacgacctggctcagcgccacgggctggagtcggcctccgaccacc |
| | atggcaacttctccatcaccatgcgcaacctgaccctgctggatagcggcctctactgctgcctggtggtgg |
| | agatcaggcaccaccactcggagcacagggtccatggtgccatggagctgcaggtgcagacaggcaaa |
| | gatgcaccatccaactgtgtggtgtacccatcctcctcccaggatagtgaaaacatcacggctgca |
| | PD-L1 (CD274), Accession Number Q9NZQ7 |
| SEQ ID NO: 39 | MRIFAVFIFMTYWHLLNAFTVTVPKDLYVVEYGSNMTIECKFPVEK |
| | QLDLAALIVYWEMEDKNIIQFVHG |
| | EEDLKVQHSSYRQRARLLKDQLSLGNAALQITDVKLQDAGVYRCM |
| | ISYGGADYKRITVKVNAPYNKINQR |
| | ILVVDPVTSEHELTCQAEGYPKAEVIWTSSDHQVLSGKTTTTNSKRE |
| | EKLFNVTSTLRINTTTNEIFYCT |
| | FRRLDPEENHTAELVIPELPLAHPPNERTHLVILGAILLCLGVALTFIF |
| | RLRKGRMMDVKKCGIQDTNSK |
| | KQSDTHLEET |
| SEQ ID NO: 40 | tttactgtcacggttcccaaggacctatatgtggtagagtatggtagcaatatgacaattgaatgcaaattccca |
| | gtagaaaaacaattagacctggctgcactaattgtctattgggaaatggaggataagaacattattcaatttgt |
| | gcatggagaggaagacctgaaggttcagcatagtagctacagacagagggcccggctgttgaaggacca |
| | gctctccctgggaaatgctgcacttcagatcacagatgtgaaattgcaggatgcaggggtgtaccgctgcat |
| | gatcagctatggtggtgccgactacaagcgaattactgtgaaagtcaatgccccatacaacaaaatcaacca |
| | aagaattttggttgtggatccagtcacctctgaacatgaactgacatgtcaggctgagggctaccccaaggc |
| | cgaagtcatctggacaagcagtgaccatcaagtcctgagtggtaagaccaccaccaccaattccaagaga |
| | gaggagaagcttttcaatgtgaccagcacactgagaatcaacacaacaactaatgagattttctactgcacttt |
| | taggagattagatcctgaggaaaaccatacagctgaattggtcatcccagaactacctctggcacatcctcca |
| | aatgaaagg |
| | CTLA-4, Accession Number P16410 |
| SEQ ID NO: 41 | MACLGFQRHKAQLNLATRTWPCTLLFFLLFIPVFCKAMHVAQPAV |
| | VLASSRGIASFVCEYASPGKATEVR |
| | VTVLRQADSQVTEVCAATYMMGNELTFLDDSICTGTSSGNQVNLTI |
| | QGLRAMDTGLYICKVELMYPPPYY |
| | LGIGNGTQIYVIDPEPCPDSDFLLWILAAVSSGLFFYSFLLTAVSLSK |
| | MLKKRSPLTTGVYVKMPPTEPE |
| | CEKQFQPYFIPIN |
| SEQ ID NO: 42 | aaagcaatgcacgtggcccagcctgctgtggtactggccagcagccgaggcatcgccagctttgtgtgtga |
| | gtatgcatctccaggcaaagccactgaggtccgggtgacagtgcttcggcaggctgacagccaggtgact |
| | gaagtctgtgcggcaacctacatgatggggaatgagttgaccttcctagatgattccatctgcacgggcacct |
| | ccagtggaaatcaagtgaacctcactatccaaggactgagggccatggacacgggactctacatctgcaag |
| | gtggagctcatgtacccaccgccatactacctgggcataggcaacggaacccagatttatgtaattgatcca |
| | gaaccgtgcccagattctgac |
| | IGSF11 (VSIG-3) |
| SEQ ID NO: 43 | MTSQRSPLAP LLLLSLHGVA ASLEVSESPG SIQVARGQPA |
| | VLPCTFTTSA ALINLNVIWM VTPLSNANQP EQVILYQGGQ |
| | MFDGAPRFHG RVGFTGTMPA TNVSIFINNT QLSDTGTYQC |
| | LVNNLPDIGG RNIGVTGLTV LVPPSAPHCQ IQGSQDIGSD |
| | VILLCSSEEG IPRPTYLWEK LDNTLKLPPT ATQDQVQGTV |
| | TIRNISALSS GLYQCVASNA IGTSTCLLDL QVISPQPRNI |
| | GLIAGAIGTG AVIIIFCIAL ILGAFFYWRS KNKEEEEEEI |
| | PNEIREDDLP PKCSSAKAFH TEISSSDNNT LTSSNAYNSR |
| | YWSNNPKVHR NTESVSHFSD LGQSFSFHSG NANIPSIYAN |
| | GTHLVPGQHK TLVVTANRGS SPQVMSRSNG SVSRKPRPPH |
| | THSYTISHAT LERIGAVPVM VPAQSRAGSL V |
| SEQ ID NO: 44 | ctggaagtgtcagagagccctgggagtatccaggtggcccggggtcagccagcagtcctgccctgcacttt |
| | cactaccagcgctgccctcattaacctcaatgtcatttggatggtcactcctctctccaatgccaaccaacctg |
| | aacaggtcatcctgtatcagggtggacagatgtttgatggtgccccccggttccacggtagggtaggatttac |
| | aggcaccatgccagctaccaatgtctctatcttcattaataacactcagttatcagacactggcacctaccagt |
| | gcctggtcaacaaccttccagacatagggggcaggaacattggggtcaccggtctcacagtgttagttccc |
| | ccttctgccccacactgccaaatccaaggatcccaggatattggcagcgatgtcatcctgctctgtagctcag |
| | aggaaggcattcctcgaccaacttacctttgggagaagttagacaataccctcaaactacctccaacagcta |
| | ctcaggaccaggtccagggaacagtcaccatccggaacatcagtgccctgtcttcaggtttgtaccagtgcg |
| | tggcttctaatgctattggaaccagcacctgtcttctggatctccaggttatttcaccccagcccaggaacattgga |

In some embodiments, the composition described herein comprises any one or more of components selected from the transmembrane moiety, the immune checkpoint moiety, the affinity receiver, the selection tag, the self-cleavage peptide, the glycosylation motif, the spacer, the gRNA and the linker. In some embodiments, the composition comprises one or more transmembrane moieties. In some embodiments, the composition comprises one or more immune checkpoint moieties (e.g., one or more first immune checkpoint moieties that are different from another one or more second immune checkpoint moities). In some embodiments, the composition comprises one or more affinity receivers. In some embodiments, the composition comprises one or more selection tags. In some embodiments, the composition comprises one or more self-cleavage peptides. In some embodiments, the composition comprises one or more glycosylation motifs. In some embodiments, the composition comprises one or more spacers. In some embodiments, the composition comprises one or more gRNAs. In some embodiments, the composition comprises one or more linkers. In some embodiments, the transmembrane moiety, the immune checkpoint moiety, the affinity receiver, the selection tag, the self-cleavage peptide, the glycosylation motif, the spacer, the gRNA and/or the linker in the composition described herein can be arranged in a different order. In some embodiments, any one or more of transmembrane moiety, the immune checkpoint moiety, the affinity receiver, the selection tag, the self-cleavage peptide, the glycosylation motif, the spacer, the gRNA, and the linker can be omitted.

In some embodiments, the composition comprises the transmembrane moiety as exemplified in Table 1. In some embodiments, the composition comprises the immune checkpoint moiety as exemplified in Table 2. In some embodiments, the composition comprises the affinity receiver as exemplified in Table 4. In some embodiments, the composition comprises the selection tag as exemplified in Table 5. In some embodiments, the composition comprises the self-cleavage peptide as exemplified in Table 6. In some embodiments, the composition comprises the glycosylation motif as exemplified in Table 7. In some embodiments, the composition comprises the spacer as exemplified in Table 8. In some embodiments, the composition comprises the gRNA as exemplified in Table 9. In some embodiments, the composition comprises the linker as exemplified in Table 10.

In some embodiments, the construct described herein is encoded by a polynucleotide sequence that is at least 50%, 55%, 60%, 65%, 70%, 75%, 80%, 85%, 90%, 95%, or 99% identical to any one of SEQ ID NOS: 4-15 and SEQ ID NOs: 45-57 (Table 3).

TABLE 3
Exemplary construct sequences

| | |
|---|---|
| | Engineered LAMP2B with immune checkpoint moiety VISTA variant 1 |
| SEQ ID NO: 4 | cttaaatggttgcccaatatttatgctgctggcatgtttgctgggatattttttctcctctgaattagtattttgagtc |
| | cttgggctcttcagtttaatagataatgtttctatgtttaagtgtagaagatccaccatgttagctgaaaagttcca |
| | aggcttttctgtctctgtaacatttctctgcctaaagaggtagcaggttgaaaatttaggaaataactttgcgtca |
| | aaaaattcatcagcctttctaatcaattcgattttaaagtgaaacagtgatgaactttgcatttatcagaattcatgt |
| | ttttctcatggatttaacagccctttgcaactctgcttccttggttacatgatagaaagagtatctgatcacatcag |
| | aataaaatcgtttgggaagacatccgtgtgtcatagattatgtagcttgcaacaggatttaaagcaaaaagatg |
| | gtagaatatcggatctatcattatttttgcagtgaatgtttcaccagctggtttgagagaagttttacacccctacc |
| | aggcttcctcttacctcatttaccgcaaaaaccacaagtcctaacaggcaaactgtaattgcctaaaacaaaa |
| | ctagtaaggtataaatgcatgtggaaatgattatttgaaggcaaaaggcaaataaatgtcaccagtctgagcc |
| | atgacctaagcatcctaggtctagtaaccattcttttggtaagggataatgccaacttttttatcaataggattctg |
| | ttaaaaatacaaagaaaaacagtcagcaggatactgtatctatacacatacatatatgtatatacacatatatgt |
| | atatatatgtatgtgtatatatagatataaaatacttttgtttcattttagagctggttgaacttcagtggtgggtaga |
| | gcttgtttttaatttatataatttttgtagtacaattttaaaatattatttttttaaatgaatccaggagccgtccgctcc |
| | tacgccttaggcaacagcaccatgttcaaggtcgccacgccgtattccctgtatgtctgtcccgaggggcag |
| | aacgtcaccctcacctgcaggctcttgggccctgtggacaaagggcacgatgtgaccttctacaagacgtg |
| | gtaccgcagctcgaggggcgaggtgcagacctgctcagagcgccggcccatccgcaacctcacgttcca |
| | ggaccttcacctgcaccatggaggccaccaggctgccaacaccagccacgacctggctcagcgccacgg |
| | gctggagtcggcctccgaccaccatggcaacttctccatcaccatgcgcaacctgaccctgctggatagcg |
| | gcctctactgctgcctggtggtggagatcaggcaccaccactcggagcacagggtccatggtgccatgga |
| | gctgcaggtgcagacaggcaaagatgcaccatccaactgtgtggtgtacccatcctcctcccaggatagtg |
| | aaaacatcacggctgcaggcggcggcggcagcgaacttaatttgacagattcagaaaatgccacttgccttt |
| | atgcaaaatggcagatgaatttcacagtacgctatgaaactacaaataaaacttatgtaagtatatattttaatttt |
| | tctttgagttttatagtaggaatttaattgactttatcctttagatacacgtatacaaatgaaattccacgttgactg |
| | acttaatgctgaaactctgggattttttttaagcatacacactcagaagttattctgtttagtgttagaatgaatattt |
| | cttctgattcattggttcttgggagtttttgtaggacttgattaagggcaaatgatctgaagacgactatatggtgt |
| | tcaggtctaagtatttctgtaaagtgtttaaaatgtactagcaccaaagcttgcccttttaaaaagcagcttccca |
| | tctgggcatggtggctcatgcctgtgatcccagcattttggaaggccaaagcaggaggattgcttgaaccca |
| | gcccgggcaacatggtgagacctcatctctacaaaaaaactttaaaaattagccaggcatggtggcacaca |
| | catgtacctccagccactcgctgggccgaggctggaggattgcttgagctcaggaggttgagtctgcagtg |
| | agccatgattgtgctactgcactccagcctgggcaacagagcgagactctatctcaaaaaaaaaaaaaatc |
| | gcttacttaggttggtacaaaagtaattgccattaaaggaacaaaccacaattacttttgcaccaacctaagta |
| | agcgatttttttttttggcacaaactgcaattacttttgcaccaacctaacactaaaatctccccctttttgacatct |
| | cctcttaattcctttgccttcttattttctttttgttaaggtcctcactacttacaacctaattttttaaaaaatcagttta |
| | agaacagcagctcaagccagttttccttttctttttagacatacacgaa |
| | Engineered LAMP2B with immune checkpoint moiety VISTA variant 2 |
| SEQ ID NO: 5 | cttaaatggttgcccaatatttatgctgctggcatgtttgctgggatattttttctcctctgaattagtattttgagtc |
| | cttgggctcttcagtttaatagataatgtttctatgtttaagtgtagaagatccaccatgttagctgaaaagttcca |
| | aggcttttctgtctctgtaacatttctctgcctaaagaggtagcaggttgaaaatttaggaaataactttgcgtca |
| | aaaaattcatcagcctttctaatcaattcgattttaaagtgaaacagtgatgaactttgcatttatcagaattcatgt |
| | ttttctcatggatttaacagccctttgcaactctgcttccttggttacatgatagaaagagtatctgatcacatcag |
| | aataaaatcgtttgggaagacatccgtgtgtcatagattatgtagcttgcaacaggatttaaagcaaaaagatg |
| | gtagaatatcggatctatcattatttttgcagtgaatgtttcaccagctggtttgagagaagttttacacccctacc |
| | aggcttcctcttacctcatttaccgcaaaaaccacaagtcctaacaggcaaactgtaattgcctaaaacaaaa |
| | ctagtaaggtataaatgcatgtggaaatgattatttgaaggcaaaaggcaaataaatgtcaccagtctgagcc |
| | atgacctaagcatcctaggtctagtaaccattcttttggtaagggataatgccaacttttttatcaataggattctg |
| | ttaaaaatacaaagaaaaacagtcagcaggatactgtatctatacacatacatatatgtatatacacatatatgt |
| | atatatatgtatgtgtatatatagatataaaatacttttgtttcattttagagctggttgaacttcagtggtgggtaga |
| | gcttgtttttaatttatataatttttgtagtacaattttaaaatattatttttttaaatgaatccaggagccgtccgctcc |
| | tacgccttaggcaacagcaccatgttcaaggtcgccacgccgtattccctgtatgtctgtcccgaggggcag |
| | aacgtcaccctcacctgcaggctcttgggccctgtggacaaagggcacgatgtgaccttctacaagacgtg |
| | gtaccgcagctcgaggggcgaggtgcagacctgctcagagcgccggcccatccgcaacctcacgttcca |
| | ggaccttcacctgcaccatggaggccaccaggctgccaacaccagccacgacctggctcagcgccacgg |
| | gctggagtcggcctccgaccaccatggcaacttctccatcaccatgcgcaacctgaccctgctggatagcg |
| | gcctctactgctgcctggtggtggagatcaggcaccaccactcggagcacagggtccatggtgccatgga |
| | gctgcaggtgcagacaggcaaagatgcaccatccaactgtgtggtgtacccatcctcctcccaggatagtg |
| | aaaacatcacggctgcaggcggcggcggcagc |
| | Engineered LAMP2B with immune checkpoint moiety VISTA variant 3 |
| SEQ ID NO: 6 | ggcaacagcaccatgttcaaggtcgccacgccgtattccctgtatgtctgtcccgaggggcagaacgtcac |
| | cctcacctgcaggctcttgggccctgtggacaaagggcacgatgtgaccttctacaagacgtggtaccgca |
| | gctcgaggggcgaggtgcagacctgctcagagcgccggcccatccgcaacctcacgttccaggaccttc |
| | acctgcaccatggaggccaccaggctgccaacaccagccacgacctggctcagcgccacgggctggag |
| | tcggcctccgaccaccatggcaacttctccatcaccatgcgcaacctgaccctgctggatagcggcctctac |
| | tgctgcctggtggtggagatcaggcaccaccactcggagcacagggtccatggtgccatggagctgcagg |
| | tgcagacaggcaaagatgcaccatccaactgtgtggtgtacccatcctcctcccaggatagtgaaaacatca |
| | cggctgcaggcggcggcggcagcgaacttaatttgacagattcagaaaatgccacttgcctttatgcaaaat |
| | ggcagatgaatttcacagtacgctatgaaactacaaataaaacttatgtaagtatatattttaatttttctttgagttt |
| | tatagtaggaatttaattgactttatcctttagatacacgtatacaaatgaaattccacgttgactgacttaatgct |
| | gaaactctgggattttttttaagcatacacactcagaagttattctgtttagtgttagaatgaatatttcttctgattc |
| | attggttcttgggagtttttgtaggacttgattaagggcaaatgatctgaagacgactatatggtgttcaggtcta |
| | agtatttctgtaaagtgtttaaaatgtactagcaccaaagcttgcccttttaaaaagcagcttcccatctgggcat |
| | ggtggctcatgcctgtgatcccagcattttggaaggccaaagcaggaggattgcttgaacccagcccgggc |
| | aacatggtgagacctcatctctacaaaaaaactttaaaaattagccaggcatggtggcacacacatgtacctc |
| | cagccactcgctgggccgaggctggaggattgcttgagctcaggaggttgagtctgcagtgagccatgatt |
| | gtgctactgcactccagcctgggcaacagagcgagactctatctcaaaaaaaaaaaaaatcgcttacttagg |
| | ttggtacaaaagtaattgccattaaaggaacaaaccacaattacttttgcaccaacctaagtaagcgatttttttt |
| | tttggcacaaactgcaattacttttgcaccaacctaacactaaaatctccccctttttgacatctcctcttaattcct |
| | ttgccttcttattttctttttgttaaggtcctcactacttacaacctaattttttaaaaaatcagtttaagaacagcagc |
| | tcaagccagttttccttttctttttagacatacacgaa |
| | Engineered LAMP2B and immune checkpoint moiety PD-L1 variant 1 |
| SEQ ID NO: 7 | cttaaatggttgcccaatatttatgctgctggcatgtttgctgggatattttttctcctctgaattagtattttgagtc |
| | cttgggctcttcagtttaatagataatgtttctatgtttaagtgtagaagatccaccatgttagctgaaaagttcca |
| | aggcttttctgtctctgtaacatttctctgcctaaagaggtagcaggttgaaaatttaggaaataactttgcgtca |
| | aaaaattcatcagcctttctaatcaattcgattttaaagtgaaacagtgatgaactttgcatttatcagaattcatgt |
| | ttttctcatggatttaacagccctttgcaactctgcttccttggttacatgatagaaagagtatctgatcacatcag |
| | aataaaatcgtttgggaagacatccgtgtgtcatagattatgtagcttgcaacaggatttaaagcaaaaagatg |
| | gtagaatatcggatctatcattatttttgcagtgaatgtttcaccagctggtttgagagaagttttacacccctacc |
| | aggcttcctcttacctcatttaccgcaaaaaccacaagtcctaacaggcaaactgtaattgcctaaaacaaaa |
| | ctagtaaggtataaatgcatgtggaaatgattatttgaaggcaaaaggcaaataaatgtcaccagtctgagcc |
| | atgacctaagcatcctaggtctagtaaccattcttttggtaagggataatgccaacttttttatcaataggattctg |
| | ttaaaaatacaaagaaaaacagtcagcaggatactgtatctatacacatacatatatgtatatacacatatatgt |
| | atatatatgtatgtgtatatatagatataaaatacttttgtttcattttagagctggttgaacttcagtggtgggtaga |
| | gcttgtttttaatttatataatttttgtagtacaattttaaaatattatttttttaaatgaatccaggagccgtccgctcc |
| | tacgccttaggcaacagcaccatgtttactgtcacggttcccaaggacctatatgtggtagagtatggtagca |
| | atatgacaattgaatgcaaattcccagtagaaaaacaattagacctggctgcactaattgtctattgggaaatg |
| | gaggataagaacattattcaatttgtgcatggagaggaagacctgaaggttcagcatagtagctacagacag |
| | agggcccggctgttgaaggaccagctctccctgggaaatgctgcacttcagatcacagatgtgaaattgca |
| | ggatgcaggggtgtaccgctgcatgatcagctatggtggtgccgactacaagcgaattactgtgaaagtca |
| | atgccccatacaacaaaatcaaccaaagaattttggttgtggatccagtcacctctgaacatgaactgacatgt |
| | caggctgagggctaccccaaggccgaagtcatctggacaagcagtgaccatcaagtcctgagtggtaaga |
| | ccaccaccaccaattccaagagagaggagaagcttttcaatgtgaccagcacactgagaatcaacacaaca |
| | actaatgagattttctactgcacttttaggagattagatcctgaggaaaaccatacagctgaattggtcatccca |
| | gaactacctctggcacatcctccaaatgaaaggggcggcggcggcagcgaacttaatttgacagattcaga |
| | aaatgccacttgcctttatgcaaaatggcagatgaatttcacagtacgctatgaaactacaaataaaacttatgt |
| | aagtatatattttaatttttctttgagttttatagtaggaatttaattgactttatcctttagatacacgtatacaaatga |
| | aattccacgttgactgacttaatgctgaaactctgggattttttttaagcatacacactcagaagttattctgtttag |
| | tgttagaatgaatatttcttctgattcattggttcttgggagtttttgtaggacttgattaagggcaaatgatctgaa |
| | gacgactatatggtgttcaggtctaagtatttctgtaaagtgtttaaaatgtactagcaccaaagcttgccctttta |
| | aaaagcagcttcccatctgggcatggtggctcatgcctgtgatcccagcattttggaaggccaaagcagga |
| | ggattgcttgaacccagcccgggcaacatggtgagacctcatctctacaaaaaaactttaaaaattagccag |
| | gcatggtggcacacacatgtacctccagccactcgctgggccgaggctggaggattgcttgagctcagga |
| | ggttgagtctgcagtgagccatgattgtgctactgcactccagcctgggcaacagagcgagactctatctca |
| | aaaaaaaaaaaaatcgcttacttaggttggtacaaaagtaattgccattaaaggaacaaaccacaattactttt |
| | gcaccaacctaagtaagcgatttttttttttggcacaaactgcaattacttttgcaccaacctaacactaaaatct |
| | ccccctttttgacatctcctcttaattcctttgccttcttattttctttttgttaaggtcctcactacttacaacctaatttt |
| | ttaaaaaatcagtttaagaacagcagctcaagccagttttcctttttttttagacatacacgaa |
| | Engineered LAMP2B and immune checkpoint moiety PD-L1 variant 2 |
| SEQ ID NO: 8 | cttaaatggttgcccaatatttatgctgctggcatgtttgctgggatattttttctcctctgaattagtattttgagtc |
| | cttgggctcttcagtttaatagataatgtttctatgtttaagtgtagaagatccaccatgttagctgaaaagttcca |
| | aggcttttctgtctctgtaacatttctctgcctaaagaggtagcaggttgaaaatttaggaaataactttgcgtca |
| | aaaaattcatcagcctttctaatcaattcgattttaaagtgaaacagtgatgaactttgcatttatcagaattcatgt |
| | ttttctcatggatttaacagccctttgcaactctgcttccttggttacatgatagaaagagtatctgatcacatcag |
| | aataaaatcgtttgggaagacatccgtgtgtcatagattatgtagcttgcaacaggatttaaagcaaaaagatg |
| | gtagaatatcggatctatcattatttttgcagtgaatgtttcaccagctggtttgagagaagttttacacccctacc |
| | aggcttcctcttacctcatttaccgcaaaaaccacaagtcctaacaggcaaactgtaattgcctaaaacaaaa |
| | ctagtaaggtataaatgcatgtggaaatgattatttgaaggcaaaaggcaaataaatgtcaccagtctgagcc |
| | atgacctaagcatcctaggtctagtaaccattcttttggtaagggataatgccaacttttttatcaataggattctg |
| | ttaaaaatacaaagaaaaacagtcagcaggatactgtatctatacacatacatatatgtatatacacatatatgt |
| | atatatatgtatgtgtatatatagatataaaatacttttgtttcattttagagctggttgaacttcagtggtgggtaga |
| | gcttgtttttaatttatataatttttgtagtacaattttaaaatattatttttttaaatgaatccaggagccgtccgctcc |
| | tacgccttaggcaacagcaccatgtttactgtcacggttcccaaggacctatatgtggtagagtatggtagca |
| | atatgacaattgaatgcaaattcccagtagaaaaacaattagacctggctgcactaattgtctattgggaaatg |
| | gaggataagaacattattcaatttgtgcatggagaggaagacctgaaggttcagcatagtagctacagacag |
| | agggcccggctgttgaaggaccagctctccctgggaaatgctgcacttcagatcacagatgtgaaattgca |
| | ggatgcaggggtgtaccgctgcatgatcagctatggtggtgccgactacaagcgaattactgtgaaagtca |
| | atgccccatacaacaaaatcaaccaaagaattttggttgtggatccagtcacctctgaacatgaactgacatgt |
| | caggctgagggctaccccaaggccgaagtcatctggacaagcagtgaccatcaagtcctgagtggtaaga |
| | ccaccaccaccaattccaagagagaggagaagcttttcaatgtgaccagcacactgagaatcaacacaaca |
| | actaatgagattttctactgcacttttaggagattagatcctgaggaaaaccatacagctgaattggtcatccca |
| | gaactacctctggcacatcctccaaatgaaaggggcggcggcggcagcgaacttaatttgacagattcaga |
| | aaatgccacttgcctttatgcaaaatggcagatgaatttcacagtacgctatgaaactacaaataaaacttatgt |
| | aagtatatattttaatttttctttgagttttatagtaggaatttaattgactttatcctttagatacacgtatacaaatga |
| | aattccacgttgactgacttaatgctgaaactctgggattttttttaagcatacacactcagaagttattctgtttag |
| | tgttagaatgaatatttcttctgattcattggttcttgggagtttttgtaggacttgattaagggcaaatgatctgaa |
| | gacgactatatggtgttcaggtctaagtatttctgtaaagtgtttaaaatgtactagcaccaaagcttgccctttta |
| | aaaagcagcttcccatctgggcatggtggctcatgcctgtgatcccagcattttggaaggccaaagcagga |
| | ggattgcttgaacccagcccgggcaacatggtgagacctcatctctacaaaaaaactttaaaaattagccag |
| | gcatggtggcacacacatgtacctccagccactcgctgggccgaggctggaggattgcttgagctcagga |
| | ggttgagtctgcagtgagccatgattgtgctactgcactccagcctgggcaacagagcgagactctatctca |
| | aaaaaaaaaaaaatcgcttacttaggttggtacaaaagtaattgccattaaaggaacaaaccacaattactttt |
| | gcaccaacctaagtaagcgatttttttttttggcacaaactgcaattacttttgcaccaacctaacactaaaatct |
| | ccccctttttgacatctcctcttaattcctttgccttcttattttctttttgttaaggtcctcactacttacaacctaatttt |
| | ttaaaaaatcagtttaagaacagcagctcaagccagttttccttttctttttagacatacacgaa |
| | Engineered LAMP2B and immune checkpoint moiety PD-L1 variant 3 |
| SEQ ID NO: 9 | ggcaacagcaccatgtttactgtcacggttcccaaggacctatatgtggtagagtatggtagcaatatgacaa |
| | ttgaatgcaaattcccagtagaaaaacaattagacctggctgcactaattgtctattgggaaatggaggataa |
| | gaacattattcaatttgtgcatggagaggaagacctgaaggttcagcatagtagctacagacagagggccc |
| | ggctgttgaaggaccagctctccctgggaaatgctgcacttcagatcacagatgtgaaattgcaggatgcag |
| | gggtgtaccgctgcatgatcagctatggtggtgccgactacaagcgaattactgtgaaagtcaatgccccat |
| | acaacaaaatcaaccaaagaattttggttgtggatccagtcacctctgaacatgaactgacatgtcaggctga |
| | gggctaccccaaggccgaagtcatctggacaagcagtgaccatcaagtcctgagtggtaagaccaccacc |
| | accaattccaagagagaggagaagcttttcaatgtgaccagcacactgagaatcaacacaacaactaatga |
| | gattttctactgcacttttaggagattagatcctgaggaaaaccatacagctgaattggtcatcccagaactac |
| | ctctggcacatcctccaaatgaaaggggcggcggcggcagcgaacttaatttgacagattcagaaaatgcc |
| | acttgcctttatgcaaaatggcagatgaatttcacagtacgctatgaaactacaaataaaacttatgtaagtatat |
| | attttaatttttctttgagttttatagtaggaatttaattgactttatcctttagatacacgtatacaaatgaaattccac |
| | gttgactgacttaatgctgaaactctgggattttttttaagcatacacactcagaagttattctgtttagtgttagaa |
| | tgaatatttcttctgattcattggttcttgggagtttttgtaggacttgattaagggcaaatgatctgaagacgact |
| | atatggtgttcaggtctaagtatttctgtaaagtgtttaaaatgtactagcaccaaagcttgcccttttaaaaagc |
| | agcttcccatctgggcatggtggctcatgcctgtgatcccagcattttggaaggccaaagcaggaggattgc |
| | ttgaacccagcccgggcaacatggtgagacctcatctctacaaaaaaactttaaaaattagccaggcatggt |
| | ggcacacacatgtacctccagccactcgctgggccgaggctggaggattgcttgagctcaggaggttgagt |
| | ctgcagtgagccatgattgtgctactgcactccagcctgggcaacagagcgagactctatctcaaaaaaaaa |
| | aaaaatcgcttacttaggttggtacaaaagtaattgccattaaaggaacaaaccacaattacttttgcaccaac |
| | ctaagtaagcgatttttttttttggcacaaactgcaattacttttgcaccaacctaacactaaaatctcccccttttt |
| | gacatctcctcttaattcctttgccttcttattttctttttgttaaggtcctcactacttacaacctaattttttaaaaaat |
| | cagtttaagaacagcagctcaagccagttttccttttctttttagacatacacgaa |
| | Engineered LAMP2B and immune checkpoint moiety IGSF11 (VSIG-3) variant 1 |
| SEQ ID NO: 10 | cttaaatggttgcccaatatttatgctgctggcatgtttgctgggatattttttctcctctgaattagtattttgagtc |
| | cttgggctcttcagtttaatagataatgtttctatgtttaagtgtagaagatccaccatgttagctgaaaagttcca |
| | aggcttttctgtctctgtaacatttctctgcctaaagaggtagcaggttgaaaatttaggaaataactttgcgtca |
| | aaaaattcatcagcctttctaatcaattcgattttaaagtgaaacagtgatgaactttgcatttatcagaattcatgt |
| | ttttctcatggatttaacagccctttgcaactctgcttccttggttacatgatagaaagagtatctgatcacatcag |
| | aataaaatcgtttgggaagacatccgtgtgtcatagattatgtagcttgcaacaggatttaaagcaaaaagatg |
| | gtagaatatcggatctatcattatttttgcagtgaatgtttcaccagctggtttgagagaagttttacacccctacc |
| | aggcttcctcttacctcatttaccgcaaaaaccacaagtcctaacaggcaaactgtaattgcctaaaacaaaa |
| | ctagtaaggtataaatgcatgtggaaatgattatttgaaggcaaaaggcaaataaatgtcaccagtctgagcc |
| | atgacctaagcatcctaggtctagtaaccattcttttggtaagggataatgccaacttttttatcaataggattctg |
| | ttaaaaatacaaagaaaaacagtcagcaggatactgtatctatacacatacatatatgtatatacacatatatgt |
| | atatatatgtatgtgtatatatagatataaaatacttttgtttcattttagagctggttgaacttcagtggtgggtaga |
| | gcttgtttttaatttatataatttttgtagtacaattttaaaatattatttttttaaatgaatccaggagccgtccgctcc |
| | tacgccttaggcaacagcaccatgctggaagtgtcagagagccctgggagtatccaggtggcccggggtc |
| | agccagcagtcctgccctgcactttcactaccagcgctgccctcattaacctcaatgtcatttggatggtcact |
| | cctctctccaatgccaaccaacctgaacaggtcatcctgtatcagggtggacagatgtttgatggtgcccccc |
| | ggttccacggtagggtaggatttacaggcaccatgccagctaccaatgtctctatcttcattaataacactcag |
| | ttatcagacactggcacctaccagtgcctggtcaacaaccttccagacatagggggcaggaacattggggt |
| | caccggtctcacagtgttagttcccccttctgccccacactgccaaatccaaggatcccaggatattggcagc |
| | gatgtcatcctgctctgtagctcagaggaaggcattcctcgaccaacttacctttgggagaagttagacaata |
| | ccctcaaactacctccaacagctactcaggaccaggtccagggaacagtcaccatccggaacatcagtgc |
| | cctgtcttcaggtttgtaccagtgcgtggcttctaatgctattggaaccagcacctgtcttctggatctccaggtt |
| | atttcaccccagcccaggaacattggaggcggcggcggcagcgaacttaatttgacagattcagaaaatgc |
| | cacttgcctttatgcaaaatggcagatgaatttcacagtacgctatgaaactacaaataaaacttatgtaagtat |
| | atattttaatttttctttgagttttatagtaggaatttaattgactttatcctttagatacacgtatacaaatgaaattcc |
| | acgttgactgacttaatgctgaaactctgggattttttttaagcatacacactcagaagttattctgtttagtgttag |
| | aatgaatatttcttctgattcattggttcttgggagtttttgtaggacttgattaagggcaaatgatctgaagacga |
| | ctatatggtgttcaggtctaagtatttctgtaaagtgtttaaaatgtactagcaccaaagcttgcccttttaaaaag |
| | cagcttcccatctgggcatggtggctcatgcctgtgatcccagcattttggaaggccaaagcaggaggattg |
| | cttgaacccagcccgggcaacatggtgagacctcatctctacaaaaaaactttaaaaattagccaggcatgg |
| | tggcacacacatgtacctccagccactcgctgggccgaggctggaggattgcttgagctcaggaggttgag |
| | tctgcagtgagccatgattgtgctactgcactccagcctgggcaacagagcgagactctatctcaaaaaaaa |
| | aaaaaatcgcttacttaggttggtacaaaagtaattgccattaaaggaacaaaccacaattacttttgcaccaa |
| | cctaagtaagcgatttttttttttggcacaaactgcaattacttttgcaccaacctaacactaaaatctccccctttt |
| | tgacatctcctcttaattcctttgccttcttattttctttttgttaaggtcctcactacttacaacctaattttttaaaaaa |
| | tcagtttaagaacagcagctcaagccagttttccttttctttttagacatacacgaa |
| | Engineered LAMP2B and immune checkpoint moiety IGSF11 (VSIG-3) variant 2 |
| SEQ ID NO: 11 | caaagaaaaacagtcagcaggatactgtatctatacacatacatatatgtatatacacatatatgtatatatatgt |
| | atgtgtatatatagatataaaatacttttgtttcattttagagctggttgaacttcagtggtgggtagagcttgttttt |
| | aatttatataatttttgtagtacaattttaaaatattatttttttaaatgaatccaggagccgtccgctcctacgcctta |
| | ggcaacagcaccatgctggaagtgtcagagagccctgggagtatccaggtggcccggggtcagccagca |
| | gtcctgccctgcactttcactaccagcgctgccctcattaacctcaatgtcatttggatggtcactcctctctcca |
| | atgccaaccaacctgaacaggtcatcctgtatcagggtggacagatgtttgatggtgccccccggttccacg |
| | gtagggtaggatttacaggcaccatgccagctaccaatgtctctatcttcattaataacactcagttatcagaca |
| | ctggcacctaccagtgcctggtcaacaaccttccagacatagggggcaggaacattggggtcaccggtctc |
| | acagtgttagttcccccttctgccccacactgccaaatccaaggatcccaggatattggcagcgatgtcatcc |
| | tgctctgtagctcagaggaaggcattcctcgaccaacttacctttgggagaagttagacaataccctcaaact |
| | acctccaacagctactcaggaccaggtccagggaacagtcaccatccggaacatcagtgccctgtcttcag |
| | gtttgtaccagtgcgtggcttctaatgctattggaaccagcacctgtcttctggatctccaggttatttcacccca |
| | gcccaggaacattggaggcggcggcggcagc |
| | Engineered LAMP2B and immune checkpoint moiety IGSF11 (VSIG-3) variant 3 |
| SEQ ID NO: 12 | ggcaacagcaccatgctggaagtgtcagagagccctgggagtatccaggtggcccggggtcagccagca |
| | gtcctgccctgcactttcactaccagcgctgccctcattaacctcaatgtcatttggatggtcactcctctctcca |
| | atgccaaccaacctgaacaggtcatcctgtatcagggtggacagatgtttgatggtgccccccggttccacg |
| | gtagggtaggatttacaggcaccatgccagctaccaatgtctctatcttcattaataacactcagttatcagaca |
| | ctggcacctaccagtgcctggtcaacaaccttccagacatagggggcaggaacattggggtcaccggtctc |
| | acagtgttagttcccccttctgccccacactgccaaatccaaggatcccaggatattggcagcgatgtcatcc |
| | tgctctgtagctcagaggaaggcattcctcgaccaacttacctttgggagaagttagacaataccctcaaact |
| | acctccaacagctactcaggaccaggtccagggaacagtcaccatccggaacatcagtgccctgtcttcag |
| | gtttgtaccagtgcgtggcttctaatgctattggaaccagcacctgtcttctggatctccaggttatttcacccca |
| | gcccaggaacattggaggcggcggcggcagcgaacttaatttgacagattcagaaaatgccacttgccttt |
| | atgcaaaatggcagatgaatttcacagtacgctatgaaactacaaataaaacttatgtaagtatatattttaatttt |
| | tctttgagttttatagtaggaatttaattgactttatcctttagatacacgtatacaaatgaaattccacgttgactg |
| | acttaatgctgaaactctgggattttttttaagcatacacactcagaagttattctgtttagtgttagaatgaatattt |
| | cttctgattcattggttcttgggagtttttgtaggacttgattaagggcaaatgatctgaagacgactatatggtgt |
| | tcaggtctaagtatttctgtaaagtgtttaaaatgtactagcaccaaagcttgcccttttaaaaagcagcttccca |
| | tctgggcatggtggctcatgcctgtgatcccagcattttggaaggccaaagcaggaggattgcttgaaccca |
| | gcccgggcaacatggtgagacctcatctctacaaaaaaactttaaaaattagccaggcatggtggcacaca |
| | catgtacctccagccactcgctgggccgaggctggaggattgcttgagctcaggaggttgagtctgcagtg |
| | agccatgattgtgctactgcactccagcctgggcaacagagcgagactctatctcaaaaaaaaaaaaaatc |
| | gcttacttaggttggtacaaaagtaattgccattaaaggaacaaaccacaattacttttgcaccaacctaagta |
| | agcgatttttttttttggcacaaactgcaattacttttgcaccaacctaacactaaaatctccccctttttgacatct |
| | cctcttaattcctttgccttcttattttctttttgttaaggtcctcactacttacaacctaattttttaaaaaatcagttta |
| | agaacagcagctcaagccagttttcctttttttttagacatacacgaa |
| | Engineered LAMP2B and immune checkpoint moiety CTLA-4 variant 1 |
| SEQ ID NO: 13 | cttaaatggttgcccaatatttatgctgctggcatgtttgctgggatattttttctcctctgaattagtattttgagtc |
| | cttgggctcttcagtttaatagataatgtttctatgtttaagtgtagaagatccaccatgttagctgaaaagttcca |
| | aggcttttctgtctctgtaacatttctctgcctaaagaggtagcaggttgaaaatttaggaaataactttgcgtca |
| | aaaaattcatcagcctttctaatcaattcgattttaaagtgaaacagtgatgaactttgcatttatcagaattcatgt |
| | ttttctcatggatttaacagccctttgcaactctgcttccttggttacatgatagaaagagtatctgatcacatcag |
| | aataaaatcgtttgggaagacatccgtgtgtcatagattatgtagcttgcaacaggatttaaagcaaaaagatg |
| | gtagaatatcggatctatcattatttttgcagtgaatgtttcaccagctggtttgagagaagttttacacccctacc |
| | aggcttcctcttacctcatttaccgcaaaaaccacaagtcctaacaggcaaactgtaattgcctaaaacaaaa |
| | ctagtaaggtataaatgcatgtggaaatgattatttgaaggcaaaaggcaaataaatgtcaccagtctgagcc |
| | atgacctaagcatcctaggtctagtaaccattcttttggtaagggataatgccaacttttttatcaataggattctg |
| | ttaaaaatacaaagaaaaacagtcagcaggatactgtatctatacacatacatatatgtatatacacatatatgt |
| | atatatatgtatgtgtatatatagatataaaatacttttgtttcattttagagctggttgaacttcagtggtgggtaga |
| | gcttgtttttaatttatataatttttgtagtacaattttaaaatattatttttttaaatgaatccaggagccgtccgctcc |
| | tacgccttaggcaacagcaccatgaaagcaatgcacgtggcccagcctgctgtggtactggccagcagcc |
| | gaggcatcgccagctttgtgtgtgagtatgcatctccaggcaaagccactgaggtccgggtgacagtgcttc |
| | ggcaggctgacagccaggtgactgaagtctgtgcggcaacctacatgatggggaatgagttgaccttccta |
| | gatgattccatctgcacgggcacctccagtggaaatcaagtgaacctcactatccaaggactgagggccat |
| | ggacacgggactctacatctgcaaggtggagctcatgtacccaccgccatactacctgggcataggcaac |
| | ggaacccagatttatgtaattgatccagaaccgtgcccagattctgacggcggcggcggcagcgaacttaa |
| | tttgacagattcagaaaatgccacttgcctttatgcaaaatggcagatgaatttcacagtacgctatgaaactac |
| | aaataaaacttatgtaagtatatattttaatttttctttgagttttatagtaggaatttaattgactttatcctttagatac |
| | acgtatacaaatgaaattccacgttgactgacttaatgctgaaactctgggattttttttaagcatacacactcag |
| | aagttattctgtttagtgttagaatgaatatttcttctgattcattggttcttgggagtttttgtaggacttgattaagg |
| | gcaaatgatctgaagacgactatatggtgttcaggtctaagtatttctgtaaagtgtttaaaatgtactagcacc |
| | aaagcttgcccttttaaaaagcagcttcccatctgggcatggtggctcatgcctgtgatcccagcattttggaa |
| | ggccaaagcaggaggattgcttgaacccagcccgggcaacatggtgagacctcatctctacaaaaaaactt |
| | taaaaattagccaggcatggtggcacacacatgtacctccagccactcgctgggccgaggctggaggattg |
| | cttgagctcaggaggttgagtctgcagtgagccatgattgtgctactgcactccagcctgggcaacagagc |
| | gagactctatctcaaaaaaaaaaaaaatcgcttacttaggttggtacaaaagtaattgccattaaaggaacaa |
| | accacaattacttttgcaccaacctaagtaagcgatttttttttttggcacaaactgcaattacttttgcaccaacc |
| | taacactaaaatctccccctttttgacatctcctcttaattcctttgccttcttattttctttttgttaaggtcctcactac |
| | ttacaacctaattttttaaaaaatcagtttaagaacagcagctcaagccagttttcctttttttttagacatacacgaa |
| | Engineered LAMP2B and immune checkpoint moiety CTLA-4 variant 2 |
| SEQ ID NO: 14 | cttaaatggttgcccaatatttatgctgctggcatgtttgctgggatattttttctcctctgaattagtattttgagtc |
| | cttgggctcttcagtttaatagataatgtttctatgtttaagtgtagaagatccaccatgttagctgaaaagttcca |
| | aggcttttctgtctctgtaacatttctctgcctaaagaggtagcaggttgaaaatttaggaaataactttgcgtca |
| | aaaaattcatcagcctttctaatcaattcgattttaaagtgaaacagtgatgaactttgcatttatcagaattcatgt |
| | ttttctcatggatttaacagccctttgcaactctgcttccttggttacatgatagaaagagtatctgatcacatcag |
| | aataaaatcgtttgggaagacatccgtgtgtcatagattatgtagcttgcaacaggatttaaagcaaaaagatg |
| | gtagaatatcggatctatcattatttttgcagtgaatgtttcaccagctggtttgagagaagttttacacccctacc |
| | aggcttcctcttacctcatttaccgcaaaaaccacaagtcctaacaggcaaactgtaattgcctaaaacaaaa |
| | ctagtaaggtataaatgcatgtggaaatgattatttgaaggcaaaaggcaaataaatgtcaccagtctgagcc |
| | atgacctaagcatcctaggtctagtaaccattcttttggtaagggataatgccaacttttttatcaataggattctg |
| | ttaaaaatacaaagaaaaacagtcagcaggatactgtatctatacacatacatatatgtatatacacatatatgt |
| | atatatatgtatgtgtatatatagatataaaatacttttgtttcattttagagctggttgaacttcagtggtgggtaga |
| | gcttgtttttaatttatataatttttgtagtacaattttaaaatattatttttttaaatgaatccaggagccgtccgctcc |
| | tacgccttaggcaacagcaccatgaaagcaatgcacgtggcccagcctgctgtggtactggccagcagcc |
| | gaggcatcgccagctttgtgtgtgagtatgcatctccaggcaaagccactgaggtccgggtgacagtgcttc |
| | ggcaggctgacagccaggtgactgaagtctgtgcggcaacctacatgatggggaatgagttgaccttccta |
| | gatgattccatctgcacgggcacctccagtggaaatcaagtgaacctcactatccaaggactgagggccat |
| | ggacacgggactctacatctgcaaggtggagctcatgtacccaccgccatactacctgggcataggcaac |
| | ggaacccagatttatgtaattgatccagaaccgtgcccagattctgac |
| | Engineered LAMP2B and immune checkpoint moiety CTLA-4 variant 3 |
| SEQ ID NO: 15 | ggcaacagcaccatgaaagcaatgcacgtggcccagcctgctgtggtactggccagcagccgaggcatc |
| | gccagctttgtgtgtgagtatgcatctccaggcaaagccactgaggtccgggtgacagtgcttcggcaggct |
| | gacagccaggtgactgaagtctgtgcggcaacctacatgatggggaatgagttgaccttcctagatgattcc |
| | atctgcacgggcacctccagtggaaatcaagtgaacctcactatccaaggactgagggccatggacacgg |
| | gactctacatctgcaaggtggagctcatgtacccaccgccatactacctgggcataggcaacggaacccag |
| | atttatgtaattgatccagaaccgtgcccagattctgacggcggcggcggcagcgaacttaatttgacagatt |
| | cagaaaatgccacttgcctttatgcaaaatggcagatgaatttcacagtacgctatgaaactacaaataaaact |
| | tatgtaagtatatattttaatttttctttgagttttatagtaggaatttaattgactttatcctttagatacacgtatacaa |
| | atgaaattccacgttgactgacttaatgctgaaactctgggattttttttaagcatacacactcagaagttattctg |
| | tttagtgttagaatgaatatttcttctgattcattggttcttgggagtttttgtaggacttgattaagggcaaatgatc |
| | tgaagacgactatatggtgttcaggtctaagtatttctgtaaagtgtttaaaatgtactagcaccaaagcttgcc |
| | cttttaaaaagcagcttcccatctgggcatggtggctcatgcctgtgatcccagcattttggaaggccaaagc |
| | aggaggattgcttgaacccagcccgggcaacatggtgagacctcatctctacaaaaaaactttaaaaattag |
| | ccaggcatggtggcacacacatgtacctccagccactcgctgggccgaggctggaggattgcttgagctc |
| | aggaggttgagtctgcagtgagccatgattgtgctactgcactccagcctgggcaacagagcgagactctat |
| | ctcaaaaaaaaaaaaaatcgcttacttaggttggtacaaaagtaattgccattaaaggaacaaaccacaatta |
| | cttttgcaccaacctaagtaagcgatttttttttttggcacaaactgcaattacttttgcaccaacctaacactaaa |
| | atctccccctttttgacatctcctcttaattcctttgccttcttattttctttttgttaaggtcctcactacttacaaccta |
| | attttttaaaaaatcagtttaagaacagcagctcaagccagttttccttttctttttagacatacacgaa |
| | Engineered PuroR plasmid comprising LAMP2B and PD-L1 variant |
| SEQ ID NO: 45 | agcagtggaatgaccgagtacaagcccacggtgcgcctcgccacccgcgacgacgtccccagggccgt |
| | acgcaccctcgccgccgcgttcgccgactaccccgccacgcgccacaccgtcgatccggaccgccacat |
| | cgagcgggtcaccgagctgcaagaactcttcctcacgcgcgtcgggctcgacatcggcaaggtgtgggtc |
| | gcggacgacggcgccgcggtggcggtctggaccacgccggagagcgtcgaagcgggggcggtgttcg |
| | ccgagatcggcccgcgcatggccgagttgagcggttcccggctggccgcgcagcaacagatggaaggc |
| | ctcctggcgccgcaccggcccaaggagcccgcgtggttcctggccaccgtcggcgtctcgcccgaccac |
| | cagggcaagggtctgggcagcgccgtcgtgctccccggagtggaggcggccgagcgcgccggggtgc |
| | ccgccttcctggagacctccgcgccccgcaacctccccttctacgagcggctcggcttcaccgtcaccgcc |
| | gacgtcgaggtgcccgaaggaccgcgcacctggtgcatgacccgcaagcccggtgccggcagcggag |
| | agggcagaggaagtcttctaacatgcggtgacgtggaggagaatcccggccctaggctcgagggcagtg |
| | gaatggtgtgcttccgcctcttcccggttccgggctcagggctcgttctggtctgcctagtcctgggagctgt |
| | gcggtcttatgcaggtaactcgactatgggcagtggatctggatccggtggctcgagttttactgtcacggttc |
| | ccaaggacctatatgtggtagagtatggtagcaatatgacaattgaatgcaaattcccagtagaaaaacaatt |
| | agacctggctgcactaattgtctattgggaaatggaggataagaacattattcaatttgtgcatggagaggaa |
| | gacctgaaggttcagcatagtagctacagacagagggcccggctgttgaaggaccagctctccctgggaa |
| | atgctgcacttcagatcacagatgtgaaattgcaggatgcaggggtgtaccgctgcatgatcagctatggtg |
| | gtgccgactacaagcgaattactgtgaaagtcaatgccccatacaacaaaatcaaccaaagaattttggttgt |
| | ggatccagtcacctctgaacatgaactgacatgtcaggctgagggctaccccaaggccgaagtcatctgga |
| | caagcagtgaccatcaagtcctgagtggtaagaccaccaccaccaattccaagagagaggagaagcttttc |
| | aatgtgaccagcacactgagaatcaacacaacaactaatgagattttctactgcacttttaggagattagatcc |
| | tgaggaaaaccatacagctgaattggtcatcccagaactacctctggcacatcctccaaatgaaaggactca |
| | cttggtaattctgggagccatcttattatgccttggtgtagcactgacattcatcttccgtttaagaaaagggag |
| | aatgatggatgtgaaaaaatgtggcatccaagatacaaactcaaagaagcaaagtgatacacatttggagg |
| | agacgggcagtggatctggatccggtggctcgagt |
| SEQ ID NO: 46 | Engineered mNeonGreen plasmid comprising LAMP2B and PD-L1 variant |
| | agcagtggaatggtgagcaagggcgaggaggataacatggcctctctcccagcgacacatgagttacac |
| | atctttggctccatcaacggtgtggactttgacatggtgggtcagggcaccggcaatccaaatgatggttatg |
| | aggagttaaacctgaagtccaccaagggtgacctccagttctccccctggattctggtccctcatatcgggta |
| | tggcttccatcagtacctgccctaccctgacgggatgtcgcctttccaggccgccatggtagatggctccgg |
| | ctaccaagtccatcgcacaatgcagtttgaagatggtgcctcccttactgttaactaccgctacacctacgag |
| | ggaagccacatcaaaggagaggcccaggtgaaggggactggtttccctgctgacggtcctgtgatgacca |
| | actcgctgaccgctgcggactggtgcaggtcgaagaagacttaccccaacgacaaaaccatcatcagtac |
| | ctttaagtggagttacaccactggaaatggcaagcgctaccggagcactgcgcggaccacctacacctttg |
| | ccaagccaatggcggctaactatctgaagaaccagccgatgtacgtgttccgtaagacggagctcaagcac |
| | tccaagaccgagctcaacttcaaggagtggcaaaaggcctttaccgatgtgatgggcatggacgagctgta |
| | caagggcagcggagagggcagaggaagtcttctaacatgcggtgacgtggaggagaatcccggcccta |
| | ggctcgagggcagtggaatggtgtgcttccgcctcttcccggttccgggctcagggctcgttctggtctgcc |
| | tagtcctgggagctgtgcggtcttatgcaggtaactcgactatgggcagtggatctggatccggtggctcga |
| | gttttactgtcacggttcccaaggacctatatgtggtagagtatggtagcaatatgacaattgaatgcaaattcc |
| | cagtagaaaaacaattagacctggctgcactaattgtctattgggaaatggaggataagaacattattcaattt |
| | gtgcatggagaggaagacctgaaggttcagcatagtagctacagacagagggcccggctgttgaaggac |
| | cagctctccctgggaaatgctgcacttcagatcacagatgtgaaattgcaggatgcaggggtgtaccgctgc |
| | atgatcagctatggtggtgccgactacaagcgaattactgtgaaagtcaatgccccatacaacaaaatcaac |
| | caaagaattttggttgtggatccagtcacctctgaacatgaactgacatgtcaggctgagggctaccccaag |
| | gccgaagtcatctggacaagcagtgaccatcaagtcctgagtggtaagaccaccaccaccaattccaaga |
| | gagaggagaagcttttcaatgtgaccagcacactgagaatcaacacaacaactaatgagattttctactgcac |
| | ttttaggagattagatcctgaggaaaaccatacagctgaattggtcatcccagaactacctctggcacatcctc |
| | caaatgaaaggactcacttggtaattctgggagccatcttattatgccttggtgtagcactgacattcatcttcc |
| | gtttaagaaaagggagaatgatggatgtgaaaaaatgtggcatccaagatacaaactcaaagaagcaaagt |
| | gatacacatttggaggagacgggcagtggatctggatccggtggctcgagt |
| | Engineered PuroR plasmid comprising PTGFR and PD-L1 variant |
| SEQ ID NO: 47 | agcagtggaatgaccgagtacaagcccacggtgcgcctcgccacccgcgacgacgtccccagggccgt |
| | acgcaccctcgccgccgcgttcgccgactaccccgccacgcgccacaccgtcgatccggaccgccacat |
| | cgagcgggtcaccgagctgcaagaactcttcctcacgcgcgtcgggctcgacatcggcaaggtgtgggtc |
| | gcggacgacggcgccgcggtggcggtctggaccacgccggagagcgtcgaagcgggggcggtgttcg |
| | ccgagatcggcccgcgcatggccgagttgagcggttcccggctggccgcgcagcaacagatggaaggc |
| | ctcctggcgccgcaccggcccaaggagcccgcgtggttcctggccaccgtcggcgtctcgcccgaccac |
| | cagggcaagggtctgggcagcgccgtcgtgctccccggagtggaggcggccgagcgcgccggggtgc |
| | ccgccttcctggagacctccgcgccccgcaacctccccttctacgagcggctcggcttcaccgtcaccgcc |
| | gacgtcgaggtgcccgaaggaccgcgcacctggtgcatgacccgcaagcccggtgccggcagcggag |
| | agggcagaggaagtcttctaacatgcggtgacgtggaggagaatcccggccctaggctcgagggcagtg |
| | gaatgtccatgaacaattccaaacagctagtgtctcctgcagctgcgcttctttcaaacacaacctgccagac |
| | ggaaggtaactcgactatgggcagtggatctggatccggtggctcgagttttactgtcacggttcccaagga |
| | cctatatgtggtagagtatggtagcaatatgacaattgaatgcaaattcccagtagaaaaacaattagacctg |
| | gctgcactaattgtctattgggaaatggaggataagaacattattcaatttgtgcatggagaggaagacctga |
| | aggttcagcatagtagctacagacagagggcccggctgttgaaggaccagctctccctgggaaatgctgc |
| | acttcagatcacagatgtgaaattgcaggatgcaggggtgtaccgctgcatgatcagctatggtggtgccga |
| | ctacaagcgaattactgtgaaagtcaatgccccatacaacaaaatcaaccaaagaattttggttgtggatcca |
| | gtcacctctgaacatgaactgacatgtcaggctgagggctaccccaaggccgaagtcatctggacaagca |
| | gtgaccatcaagtcctgagtggtaagaccaccaccaccaattccaagagagaggagaagcttttcaatgtg |
| | accagcacactgagaatcaacacaacaactaatgagattttctactgcacttttaggagattagatcctgagg |
| | aaaaccatacagctgaattggtcatcccagaactacctctggcacatcctccaaatgaaaggactcacttggt |
| | aattctgggagccatcttattatgccttggtgtagcactgacattcatcttccgtttaagaaaagggagaatgat |
| | ggatgtgaaaaaatgtggcatccaagatacaaactcaaagaagcaaagtgatacacatttggaggagacg |
| | ggcagtggatctggatccggtggctcgagt |
| | Engineered mNeonGreen plasmid comprising PTGFR and PD-L1 variant |
| SEQ ID NO: 48 | agcagtggaatggtgagcaagggcgaggaggataacatggcctctctcccagcgacacatgagttacac |
| | atctttggctccatcaacggtgtggactttgacatggtgggtcagggcaccggcaatccaaatgatggttatg |
| | aggagttaaacctgaagtccaccaagggtgacctccagttctccccctggattctggtccctcatatcgggta |
| | tggcttccatcagtacctgccctaccctgacgggatgtcgcctttccaggccgccatggtagatggctccgg |
| | ctaccaagtccatcgcacaatgcagtttgaagatggtgcctcccttactgttaactaccgctacacctacgag |
| | ggaagccacatcaaaggagaggcccaggtgaaggggactggtttccctgctgacggtcctgtgatgacca |
| | actcgctgaccgctgcggactggtgcaggtcgaagaagacttaccccaacgacaaaaccatcatcagtac |
| | ctttaagtggagttacaccactggaaatggcaagcgctaccggagcactgcgcggaccacctacacctttg |
| | ccaagccaatggcggctaactatctgaagaaccagccgatgtacgtgttccgtaagacggagctcaagcac |
| | tccaagaccgagctcaacttcaaggagtggcaaaaggcctttaccgatgtgatgggcatggacgagctgta |
| | caagggcagcggagagggcagaggaagtcttctaacatgcggtgacgtggaggagaatcccggcccta |
| | ggctcgagggcagtggaatgtccatgaacaattccaaacagctagtgtctcctgcagctgcgcttctttcaaa |
| | cacaacctgccagacggaaggtaactcgactatgggcagtggatctggatccggtggctcgagttttactgt |
| | cacggttcccaaggacctatatgtggtagagtatggtagcaatatgacaattgaatgcaaattcccagtagaa |
| | aaacaattagacctggctgcactaattgtctattgggaaatggaggataagaacattattcaatttgtgcatgg |
| | agaggaagacctgaaggttcagcatagtagctacagacagagggcccggctgttgaaggaccagctctcc |
| | ctgggaaatgctgcacttcagatcacagatgtgaaattgcaggatgcaggggtgtaccgctgcatgatcagc |
| | tatggtggtgccgactacaagcgaattactgtgaaagtcaatgccccatacaacaaaatcaaccaaagaattt |
| | tggttgtggatccagtcacctctgaacatgaactgacatgtcaggctgagggctaccccaaggccgaagtc |
| | atctggacaagcagtgaccatcaagtcctgagtggtaagaccaccaccaccaattccaagagagaggaga |
| | agcttttcaatgtgaccagcacactgagaatcaacacaacaactaatgagattttctactgcacttttaggagat |
| | tagatcctgaggaaaaccatacagctgaattggtcatcccagaactacctctggcacatcctccaaatgaaa |
| | ggactcacttggtaattctgggagccatcttattatgccttggtgtagcactgacattcatcttccgtttaagaaa |
| | agggagaatgatggatgtgaaaaaatgtggcatccaagatacaaactcaaagaagcaaagtgatacacatt |
| | tggaggagacgggcagtggatctggatccggtggctcgagt |
| | Engineered PuroR plasmid comprising IGSF8 and PD-L1 variant |
| SEQ ID NO: 49 | agcagtggaatgaccgagtacaagcccacggtgcgcctcgccacccgcgacgacgtccccagggccgt |
| | acgcaccctcgccgccgcgttcgccgactaccccgccacgcgccacaccgtcgatccggaccgccacat |
| | cgagcgggtcaccgagctgcaagaactcttcctcacgcgcgtcgggctcgacatcggcaaggtgtgggtc |
| | gcggacgacggcgccgcggtggcggtctggaccacgccggagagcgtcgaagcgggggcggtgttcg |
| | ccgagatcggcccgcgcatggccgagttgagcggttcccggctggccgcgcagcaacagatggaaggc |
| | ctcctggcgccgcaccggcccaaggagcccgcgtggttcctggccaccgtcggcgtctcgcccgaccac |
| | cagggcaagggtctgggcagcgccgtcgtgctccccggagtggaggcggccgagcgcgccggggtgc |
| | ccgccttcctggagacctccgcgccccgcaacctccccttctacgagcggctcggcttcaccgtcaccgcc |
| | gacgtcgaggtgcccgaaggaccgcgcacctggtgcatgacccgcaagcccggtgccggcagcggag |
| | agggcagaggaagtcttctaacatgcggtgacgtggaggagaatcccggccctaggctcgagggcagtg |
| | gaatgggcgccctcaggcccacgctgctgccgccttcgctgccgctgctgctgctgctaatgctaggaatg |
| | ggatgctgggccggtaactcgactatgggcagtggatctggatccggtggctcgagttttactgtcacggttc |
| | ccaaggacctatatgtggtagagtatggtagcaatatgacaattgaatgcaaattcccagtagaaaaacaatt |
| | agacctggctgcactaattgtctattgggaaatggaggataagaacattattcaatttgtgcatggagaggaa |
| | gacctgaaggttcagcatagtagctacagacagagggcccggctgttgaaggaccagctctccctgggaa |
| | atgctgcacttcagatcacagatgtgaaattgcaggatgcaggggtgtaccgctgcatgatcagctatggtg |
| | gtgccgactacaagcgaattactgtgaaagtcaatgccccatacaacaaaatcaaccaaagaattttggttgt |
| | ggatccagtcacctctgaacatgaactgacatgtcaggctgagggctaccccaaggccgaagtcatctgga |
| | caagcagtgaccatcaagtcctgagtggtaagaccaccaccaccaattccaagagagaggagaagcttttc |
| | aatgtgaccagcacactgagaatcaacacaacaactaatgagattttctactgcacttttaggagattagatcc |
| | tgaggaaaaccatacagctgaattggtcatcccagaactacctctggcacatcctccaaatgaaaggactca |
| | cttggtaattctgggagccatcttattatgccttggtgtagcactgacattcatcttccgtttaagaaaagggag |
| | aatgatggatgtgaaaaaatgtggcatccaagatacaaactcaaagaagcaaagtgatacacatttggagg |
| | agacgggcagtggatctggatccggtggctcgagt |
| | Engineered mNeoGreen plasmid comprising IGSF8 and PD-L1 variant |
| SEQ ID NO: 50 | agcagtggaatggtgagcaagggcgaggaggataacatggcctctctcccagcgacacatgagttacac |
| | atctttggctccatcaacggtgtggactttgacatggtgggtcagggcaccggcaatccaaatgatggttatg |
| | aggagttaaacctgaagtccaccaagggtgacctccagttctccccctggattctggtccctcatatcgggta |
| | tggcttccatcagtacctgccctaccctgacgggatgtcgcctttccaggccgccatggtagatggctccgg |
| | ctaccaagtccatcgcacaatgcagtttgaagatggtgcctcccttactgttaactaccgctacacctacgag |
| | ggaagccacatcaaaggagaggcccaggtgaaggggactggtttccctgctgacggtcctgtgatgacca |
| | actcgctgaccgctgcggactggtgcaggtcgaagaagacttaccccaacgacaaaaccatcatcagtac |
| | ctttaagtggagttacaccactggaaatggcaagcgctaccggagcactgcgcggaccacctacacctttg |
| | ccaagccaatggcggctaactatctgaagaaccagccgatgtacgtgttccgtaagacggagctcaagcac |
| | tccaagaccgagctcaacttcaaggagtggcaaaaggcctttaccgatgtgatgggcatggacgagctgta |
| | caagggcagcggagagggcagaggaagtcttctaacatgcggtgacgtggaggagaatcccggcccta |
| | ggctcgagggcagtggaatgggcgccctcaggcccacgctgctgccgccttcgctgccgctgctgctgct |
| | gctaatgctaggaatgggatgctgggccggtaactcgactatgggcagtggatctggatccggtggctcga |
| | gttttactgtcacggttcccaaggacctatatgtggtagagtatggtagcaatatgacaattgaatgcaaattcc |
| | cagtagaaaaacaattagacctggctgcactaattgtctattgggaaatggaggataagaacattattcaattt |
| | gtgcatggagaggaagacctgaaggttcagcatagtagctacagacagagggcccggctgttgaaggac |
| | cagctctccctgggaaatgctgcacttcagatcacagatgtgaaattgcaggatgcaggggtgtaccgctgc |
| | atgatcagctatggtggtgccgactacaagcgaattactgtgaaagtcaatgccccatacaacaaaatcaac |
| | caaagaattttggttgtggatccagtcacctctgaacatgaactgacatgtcaggctgagggctaccccaag |
| | gccgaagtcatctggacaagcagtgaccatcaagtcctgagtggtaagaccaccaccaccaattccaaga |
| | gagaggagaagcttttcaatgtgaccagcacactgagaatcaacacaacaactaatgagattttctactgcac |
| | ttttaggagattagatcctgaggaaaaccatacagctgaattggtcatcccagaactacctctggcacatcctc |
| | caaatgaaaggactcacttggtaattctgggagccatcttattatgccttggtgtagcactgacattcatcttcc |
| | gtttaagaaaagggagaatgatggatgtgaaaaaatgtggcatccaagatacaaactcaaagaagcaaagt |
| | gatacacatttggaggagacgggcagtggatctggatccggtggctcgagt |
| | Engineered PuroR plasmid comprising LAMP2B and affinity receiver variant |
| SEQ ID NO: 51 | agcagtggaatgaccgagtacaagcccacggtgcgcctcgccacccgcgacgacgtccccagggccgt |
| | acgcaccctcgccgccgcgttcgccgactaccccgccacgcgccacaccgtcgatccggaccgccacat |
| | cgagcgggtcaccgagctgcaagaactcttcctcacgcgcgtcgggctcgacatcggcaaggtgtgggtc |
| | gcggacgacggcgccgcggtggcggtctggaccacgccggagagcgtcgaagcgggggcggtgttcg |
| | ccgagatcggcccgcgcatggccgagttgagcggttcccggctggccgcgcagcaacagatggaaggc |
| | ctcctggcgccgcaccggcccaaggagcccgcgtggttcctggccaccgtcggcgtctcgcccgaccac |
| | cagggcaagggtctgggcagcgccgtcgtgctccccggagtggaggcggccgagcgcgccggggtgc |
| | ccgccttcctggagacctccgcgccccgcaacctccccttctacgagcggctcggcttcaccgtcaccgcc |
| | gacgtcgaggtgcccgaaggaccgcgcacctggtgcatgacccgcaagcccggtgccggcagcggag |
| | agggcagaggaagtcttctaacatgcggtgacgtggaggagaatcccggccctaggctcgagggcagtg |
| | gaatggtgtgcttccgcctcttcccggttccgggctcagggctcgttctggtctgcctagtcctgggagctgt |
| | gcggtcttatgcaggtaactcgactatgggcagtggatctggatccggtggctcgagtatggtaaccacctta |
| | tcaggtttatcaggtgagcaaggtccgtccggtgatatgacaactgaagaagatagtgctacccatattaaatt |
| | ctcaaaacgtgatgaggacggccgtgagttagctggtgcaactatggagttgcgtgattcatctggtaaaact |
| | attagtacatggatttcagatggacatgtgaaggatttctacctgtatccaggaaaatatacatttgtcgaaacc |
| | gcagcaccagacggttatgaggtagcaactccaattgaatttacagttaatgaggacggtcaggttactgtag |
| | atggtgaagcaactgaaggtgacgctcatactggatccagtggtagcggcagtggatctggatccggtggc |
| | tcgagt |
| | Engineered PuroR plasmid comprising LAMP2B and PD-L1 variant |
| SEQ ID NO: 52 | agcagtggaatgaccgagtacaagcccacggtgcgcctcgccacccgcgacgacgtccccagggccgt |
| | acgcaccctcgccgccgcgttcgccgactaccccgccacgcgccacaccgtcgatccggaccgccacat |
| | cgagcgggtcaccgagctgcaagaactcttcctcacgcgcgtcgggctcgacatcggcaaggtgtgggtc |
| | gcggacgacggcgccgcggtggcggtctggaccacgccggagagcgtcgaagcgggggcggtgttcg |
| | ccgagatcggcccgcgcatggccgagttgagcggttcccggctggccgcgcagcaacagatggaaggc |
| | ctcctggcgccgcaccggcccaaggagcccgcgtggttcctggccaccgtcggcgtctcgcccgaccac |
| | cagggcaagggtctgggcagcgccgtcgtgctccccggagtggaggcggccgagcgcgccggggtgc |
| | ccgccttcctggagacctccgcgccccgcaacctccccttctacgagcggctcggcttcaccgtcaccgcc |
| | gacgtcgaggtgcccgaaggaccgcgcacctggtgcatgacccgcaagcccggtgccggcagcggag |
| | agggcagaggaagtcttctaacatgcggtgacgtggaggagaatcccggccctaggctcgagggcagtg |
| | gaatggtgtgcttccgcctcttcccggttccgggctcagggctcgttctggtctgcctagtcctgggagctgt |
| | gcggtcttatgcaggtaactcgactatgggcagtggatctggatccggtggctcgagttttactgtcacggttc |
| | ccaaggacctatatgtggtagagtatggtagcaatatgacaattgaatgcaaattcccagtagaaaaacaatt |
| | agacctggctgcactaattgtctattgggaaatggaggataagaacattattcaatttgtgcatggagaggaa |
| | gacctgaaggttcagcatagtagctacagacagagggcccggctgttgaaggaccagctctccctgggaa |
| | atgctgcacttcagatcacagatgtgaaattgcaggatgcaggggtgtaccgctgcatgatcagctatggtg |
| | gtgccgactacaagcgaattactgtgaaagtcaatgccccatacaacaaaatcaaccaaagaattttggttgt |
| | ggatccagtcacctctgaacatgaactgacatgtcaggctgagggctaccccaaggccgaagtcatctgga |
| | caagcagtgaccatcaagtcctgagtggtaagaccaccaccaccaattccaagagagaggagaagcttttc |
| | aatgtgaccagcacactgagaatcaacacaacaactaatgagattttctactgcacttttaggagattagatcc |
| | tgaggaaaaccatacagctgaattggtcatcccagaactacctctggcacatcctccaaatgaaaggactca |
| | cttggtaattctgggagccatcttattatgccttggtgtagcactgacattcatcttccgtttaagaaaagggag |
| | aatgatggatgtgaaaaaatgtggcatccaagatacaaactcaaagaagcaaagtgatacacatttggagg |
| | agacgggcagtggatctggatccggtggctcgagt |
| | Engineered mNeonGreen plasmid comprising LAMP2B and affinity receiver variant |
| SEQ ID NO: 53 | agcagtggaatggtgagcaagggcgaggaggataacatggcctctctcccagcgacacatgagttacaca |
| | tctttggctccatcaacggtgtggactttgacatggtgggtcagggcaccggcaatccaaatgatggttatga |
| | ggagttaaacctgaagtccaccaagggtgacctccagttctccccctggattctggtccctcatatcgggtat |
| | ggcttccatcagtacctgccctaccctgacgggatgtcgcctttccaggccgccatggtagatggctccggc |
| | taccaagtccatcgcacaatgcagtttgaagatggtgcctcccttactgttaactaccgctacacctacgagg |
| | gaagccacatcaaaggagaggcccaggtgaaggggactggtttccctgctgacggtcctgtgatgaccaa |
| | ctcgctgaccgctgcggactggtgcaggtcgaagaagacttaccccaacgacaaaaccatcatcagtacct |
| | ttaagtggagttacaccactggaaatggcaagcgctaccggagcactgcgcggaccacctacacctttgcc |
| | aagccaatggcggctaactatctgaagaaccagccgatgtacgtgttccgtaagacggagctcaagcactc |
| | caagaccgagctcaacttcaaggagtggcaaaaggcctttaccgatgtgatgggcatggacgagctgtaca |
| | agggcagcggagagggcagaggaagtcttctaacatgcggtgacgtggaggagaatcccggccctagg |
| | ctcgagggcagtggaatggtgtgcttccgcctcttcccggttccgggctcagggctcgttctggtctgcctag |
| | tcctgggagctgtgcggtcttatgcaggtaactcgactatgggcagtggatctggatccggtggctcgagtat |
| | ggtaaccaccttatcaggtttatcaggtgagcaaggtccgtccggtgatatgacaactgaagaagatagtgct |
| | acccatattaaattctcaaaacgtgatgaggacggccgtgagttagctggtgcaactatggagttgcgtgatt |
| | catctggtaaaactattagtacatggatttcagatggacatgtgaaggatttctacctgtatccaggaaaatata |
| | catttgtcgaaaccgcagcaccagacggttatgaggtagcaactccaattgaatttacagttaatgaggacg |
| | gtcaggttactgtagatggtgaagcaactgaaggtgacgctcatactggatccagtggtagcggcagtggat |
| | ctggatccggtggctcgagt |
| | Engineered PuroR plasmid comprising PTGFR and affinity receiver variant |
| SEQ ID NO: 54 | agcagtggaatgaccgagtacaagcccacggtgcgcctcgccacccgcgacgacgtccccagggccgt |
| | acgcaccctcgccgccgcgttcgccgactaccccgccacgcgccacaccgtcgatccggaccgccacat |
| | cgagcgggtcaccgagctgcaagaactcttcctcacgcgcgtcgggctcgacatcggcaaggtgtgggtc |
| | gcggacgacggcgccgcggtggcggtctggaccacgccggagagcgtcgaagcgggggcggtgttcg |
| | ccgagatcggcccgcgcatggccgagttgagcggttcccggctggccgcgcagcaacagatggaaggc |
| | ctcctggcgccgcaccggcccaaggagcccgcgtggttcctggccaccgtcggcgtctcgcccgaccac |
| | cagggcaagggtctgggcagcgccgtcgtgctccccggagtggaggcggccgagcgcgccggggtgc |
| | ccgccttcctggagacctccgcgccccgcaacctccccttctacgagcggctcggcttcaccgtcaccgcc |
| | gacgtcgaggtgcccgaaggaccgcgcacctggtgcatgacccgcaagcccggtgccggcagcggag |
| | agggcagaggaagtcttctaacatgcggtgacgtggaggagaatcccggccctaggctcgagggcagtg |
| | gaatgtccatgaacaattccaaacagctagtgtctcctgcagctgcgcttctttcaaacacaacctgccagac |
| | ggaaggtaactcgactatgggcagtggatctggatccggtggctcgagtatggtaaccaccttatcaggttta |
| | tcaggtgagcaaggtccgtccggtgatatgacaactgaagaagatagtgctacccatattaaattctcaaaac |
| | gtgatgaggacggccgtgagttagctggtgcaactatggagttgcgtgattcatctggtaaaactattagtac |
| | atggatttcagatggacatgtgaaggatttctacctgtatccaggaaaatatacatttgtcgaaaccgcagcac |
| | cagacggttatgaggtagcaactccaattgaatttacagttaatgaggacggtcaggttactgtagatggtga |
| | agcaactgaaggtgacgctcatactggatccagtggtagcggcagtggatctggatccggtggctcgagt |
| | Engineered mNeonGreen plasmid comprising PTGFR and affinity receiver variant |
| SEQ ID NO: 55 | agcagtggaatggtgagcaagggcgaggaggataacatggcctctctcccagcgacacatgagttacaca |
| | tctttggctccatcaacggtgtggactttgacatggtgggtcagggcaccggcaatccaaatgatggttatga |
| | ggagttaaacctgaagtccaccaagggtgacctccagttctccccctggattctggtccctcatatcgggtat |
| | ggcttccatcagtacctgccctaccctgacgggatgtcgcctttccaggccgccatggtagatggctccggc |
| | taccaagtccatcgcacaatgcagtttgaagatggtgcctcccttactgttaactaccgctacacctacgagg |
| | gaagccacatcaaaggagaggcccaggtgaaggggactggtttccctgctgacggtcctgtgatgaccaa |
| | ctcgctgaccgctgcggactggtgcaggtcgaagaagacttaccccaacgacaaaaccatcatcagtacct |
| | ttaagtggagttacaccactggaaatggcaagcgctaccggagcactgcgcggaccacctacacctttgcc |
| | aagccaatggcggctaactatctgaagaaccagccgatgtacgtgttccgtaagacggagctcaagcactc |
| | caagaccgagctcaacttcaaggagtggcaaaaggcctttaccgatgtgatgggcatggacgagctgtaca |
| | agggcagcggagagggcagaggaagtcttctaacatgcggtgacgtggaggagaatcccggccctagg |
| | ctcgagggcagtggaatgtccatgaacaattccaaacagctagtgtctcctgcagctgcgcttctttcaaaca |
| | caacctgccagacggaaggtaactcgactatgggcagtggatctggatccggtggctcgagtatggtaacc |
| | accttatcaggtttatcaggtgagcaaggtccgtccggtgatatgacaactgaagaagatagtgctacccata |
| | ttaaattctcaaaacgtgatgaggacggccgtgagttagctggtgcaactatggagttgcgtgattcatctggt |
| | aaaactattagtacatggatttcagatggacatgtgaaggatttctacctgtatccaggaaaatatacatttgtc |
| | gaaaccgcagcaccagacggttatgaggtagcaactccaattgaatttacagttaatgaggacggtcaggtt |
| | actgtagatggtgaagcaactgaaggtgacgctcatactggatccagtggtagcggcagtggatctggatc |
| | cggtggctcgagt |
| | Engineered PuroR plasmid comprising IGSF8 and affinity receiver variant |
| SEQ ID NO: 56 | agcagtggaatgaccgagtacaagcccacggtgcgcctcgccacccgcgacgacgtccccagggccgt |
| | acgcaccctcgccgccgcgttcgccgactaccccgccacgcgccacaccgtcgatccggaccgccacat |
| | cgagcgggtcaccgagctgcaagaactcttcctcacgcgcgtcgggctcgacatcggcaaggtgtgggtc |
| | gcggacgacggcgccgcggtggcggtctggaccacgccggagagcgtcgaagcgggggcggtgttcg |
| | ccgagatcggcccgcgcatggccgagttgagcggttcccggctggccgcgcagcaacagatggaaggc |
| | ctcctggcgccgcaccggcccaaggagcccgcgtggttcctggccaccgtcggcgtctcgcccgaccac |
| | cagggcaagggtctgggcagcgccgtcgtgctccccggagtggaggcggccgagcgcgccggggtgc |
| | ccgccttcctggagacctccgcgccccgcaacctccccttctacgagcggctcggcttcaccgtcaccgcc |
| | gacgtcgaggtgcccgaaggaccgcgcacctggtgcatgacccgcaagcccggtgccggcagcggag |
| | agggcagaggaagtcttctaacatgcggtgacgtggaggagaatcccggccctaggctcgagggcagtg |
| | gaatgggcgccctcaggcccacgctgctgccgccttcgctgccgctgctgctgctgctaatgctaggaatg |
| | ggatgctgggccggtaactcgactatgggcagtggatctggatccggtggctcgagtatggtaaccacctta |
| | tcaggtttatcaggtgagcaaggtccgtccggtgatatgacaactgaagaagatagtgctacccatattaaatt |
| | ctcaaaacgtgatgaggacggccgtgagttagctggtgcaactatggagttgcgtgattcatctggtaaaact |
| | attagtacatggatttcagatggacatgtgaaggatttctacctgtatccaggaaaatatacatttgtcgaaacc |
| | gcagcaccagacggttatgaggtagcaactccaattgaatttacagttaatgaggacggtcaggttactgtag |
| | atggtgaagcaactgaaggtgacgctcatactggatccagtggtagcggcagtggatctggatccggtggc |
| | tcgagt |
| | Engineered mNeoGreen plasmid comprising IGSF8 and affinity receiver variant |
| SEQ ID NO: 57 | agcagtggaatggtgagcaagggcgaggaggataacatggcctctctcccagcgacacatgagttacaca |
| | tctttggctccatcaacggtgtggactttgacatggtgggtcagggcaccggcaatccaaatgatggttatga |
| | ggagttaaacctgaagtccaccaagggtgacctccagttctccccctggattctggtccctcatatcgggtat |
| | ggcttccatcagtacctgccctaccctgacgggatgtcgcctttccaggccgccatggtagatggctccggc |
| | taccaagtccatcgcacaatgcagtttgaagatggtgcctcccttactgttaactaccgctacacctacgagg |
| | gaagccacatcaaaggagaggcccaggtgaaggggactggtttccctgctgacggtcctgtgatgaccaa |
| | ctcgctgaccgctgcggactggtgcaggtcgaagaagacttaccccaacgacaaaaccatcatcagtacct |
| | ttaagtggagttacaccactggaaatggcaagcgctaccggagcactgcgcggaccacctacacctttgcc |
| | aagccaatggcggctaactatctgaagaaccagccgatgtacgtgttccgtaagacggagctcaagcactc |
| | caagaccgagctcaacttcaaggagtggcaaaaggcctttaccgatgtgatgggcatggacgagctgtaca |
| | agggcagcggagagggcagaggaagtcttctaacatgcggtgacgtggaggagaatcccggccctagg |
| | ctcgagggcagtggaatgggcgccctcaggcccacgctgctgccgccttcgctgccgctgctgctgctgct |
| | aatgctaggaatgggatgctgggccggtaactcgactatgggcagtggatctggatccggtggctcgagta |
| | tggtaaccaccttatcaggtttatcaggtgagcaaggtccgtccggtgatatgacaactgaagaagatagtgc |
| | tacccatattaaattctcaaaacgtgatgaggacggccgtgagttagctggtgcaactatggagttgcgtgatt |
| | catctggtaaaactattagtacatggatttcagatggacatgtgaaggatttctacctgtatccaggaaaatata |
| | catttgtcgaaaccgcagcaccagacggttatgaggtagcaactccaattgaatttacagttaatgaggacg |
| | gtcaggttactgtagatggtgaagcaactgaaggtgacgctcatactggatccagtggtagcggcagtggat |
| | ctggatccggtggctcgagt |

In some embodiments, the construct is encoded by a polynucleotide sequence that is at least 50%, 55%, 60%, 65%, 70%, 75%, 80%, 85%, 90%, 95%, or 99% identical to SEQ ID NO: 4 (Table 3). In some embodiments, the construct is encoded by a polynucleotide sequence that is at least 50%, 55%, 60%, 65%, 70%, 75%, 80%, 85%, 90%, 95%, or 99% identical to SEQ ID NO: 5 (Table 3). In some embodiments, the construct is encoded by a polynucleotide sequence that is at least 50%, 55%, 60%, 65%, 70%, 75%, 80%, 85%, 90%, 95%, or 99% identical to SEQ ID NO: 6 (Table 3). In some embodiments, the construct is encoded by a polynucleotide sequence that is at least 50%, 55%, 60%, 65%, 70%, 75%, 80%, 85%, 90%, 95%, or 99% identical to SEQ ID NO: 7 (Table 3). In some embodiments, the construct is encoded by a polynucleotide sequence that is at least 50%, 55%, 60%, 65%, 70%, 75%, 80%, 85%, 90%, 95%, or 99% identical to SEQ ID NO: 8 (Table 3). In some embodiments, the construct is encoded by a polynucleotide sequence that is at least 50%, 55%, 60%, 65%, 70%, 75%, 80%, 85%, 90%, 95%, or 99% identical to SEQ ID NO: 9 (Table 3). In some embodiments, the construct is encoded by a polynucleotide sequence that is at least 50%, 55%, 60%, 65%, 70%, 75%, 80%, 85%, 90%, 95%, or 99% identical to SEQ ID NO: 10 (Table 3). In some embodiments, the construct is encoded by a polynucleotide sequence that is at least 50%, 55%, 60%, 65%, 70%, 75%, 80%, 85%, 90%, 95%, or 99% identical to SEQ ID NO: 11 (Table 3). In some embodiments, the construct is encoded by a polynucleotide sequence that is at least 50%, 55%, 60%, 65%, 70%, 75%, 80%, 85%, 90%, 95%, or 99% identical to SEQ ID NO: 12 (Table 3). In some embodiments, the construct is encoded by a polynucleotide sequence that is at least 50%, 55%, 60%, 65%, 70%, 75%, 80%, 85%, 90%, 95%, or 99% identical to SEQ ID NO: 13 (Table 3). In some embodiments, the construct is encoded by a polynucleotide sequence that is at least 50%, 55%, 60%, 65%, 70%, 75%, 80%, 85%, 90%, 95%, or 99% identical to SEQ ID NO: 14 (Table 3). In some embodiments, the construct is encoded by a polynucleotide sequence that is at least 50%, 55%, 60%, 65%, 70%, 75%, 80%, 85%, 90%, 95%, or 99% identical to SEQ ID NO: 15 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 45 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 46 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 47 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 48 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 49 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 50 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 51 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 52 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 53 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 54 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 55 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 56 (Table 3). In some embodiments, the construct comprises a sequence that is at least 10%, 20%, 30%, 40%, 50%, 60%, 70%, 75%, 80%, 85%, 90%, 95%, 99% or 100% homologous to the SEQ ID NO: 57 (Table 3).

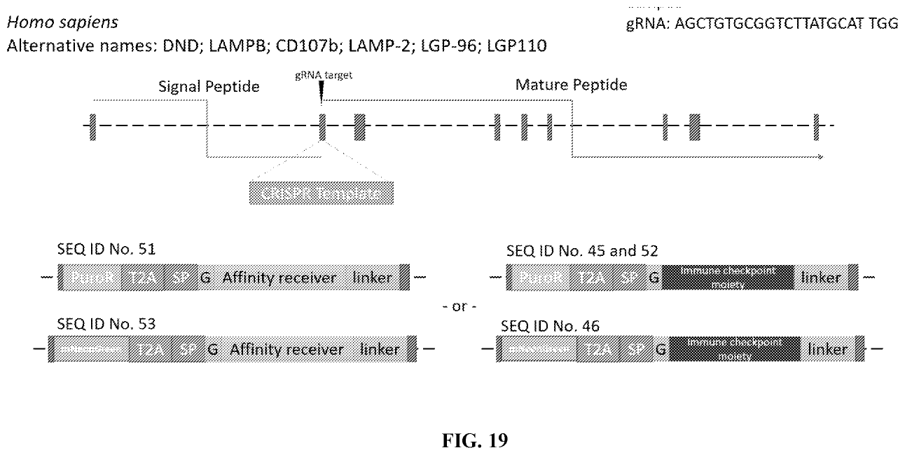

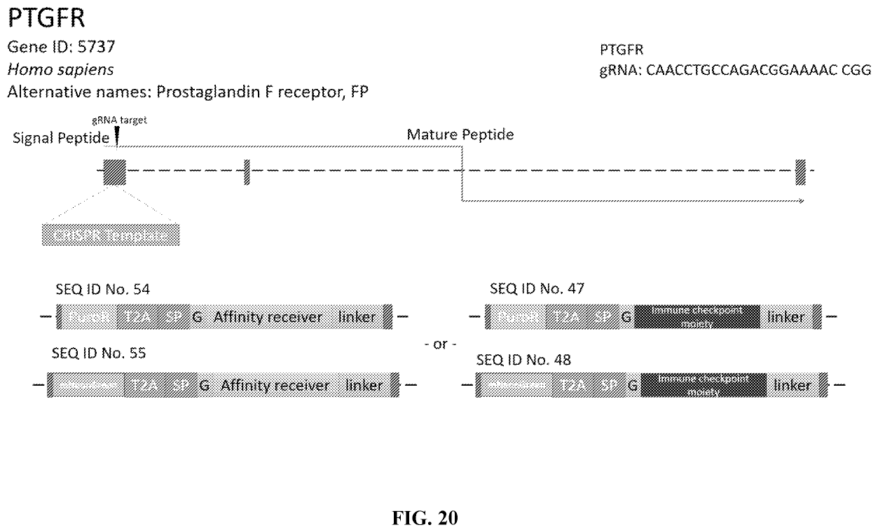

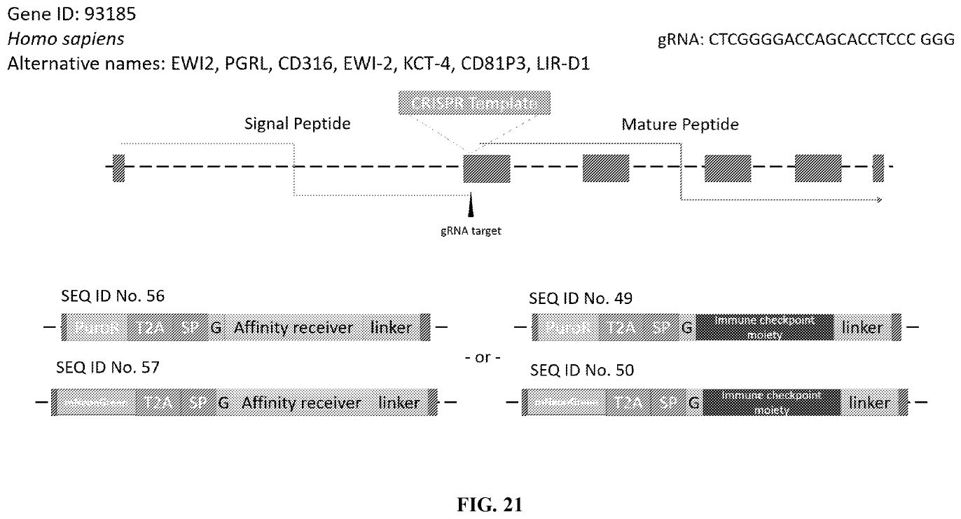

FIG. 19 demonstrates exemplary constructs that may be used as a CRISPR template for LAMP2B using SEQ ID No. 51, SEQ ID No. 45, SEQ ID No.52, SEQ ID No. 53, and SEQ ID No.46. FIG. 20 shows exemplary constructs that may be used as a CRISPR template for PTGFR using SEQ ID No. 54, SEQ ID No. 47, SEQ ID No. 55, and SEQ ID No. 48. FIG. 21 shows exemplary constructs that may be used as a CRISPR template for IGSF8 using SEQ ID No. 56, SEQ ID No. 49, SEQ ID No. 57, and SEQ ID No. 50.

In some embodiments, the immune checkpoint moiety comprises VISTA and is encoded from polynucleotide or a variation thereof or a fragment thereof that is at least 50%, 55%, 60%, 65%, 70%, 75%, 80%, 85%, 90%, 95%, or 99% identical to any one of SEQ ID NOS: 37-38 (Table 2). In some embodiments, the immune checkpoint moiety comprises the extracellular domain of VISTA. In some embodiments, the immune checkpoint moiety is the extracellular domain of VISTA. In some embodiments, the immune checkpoint moiety is PD-L1 and encoded from polynucleotide or a variation thereof or a fragment thereof that is at least 50%, 55%, 60%, 65%, 70%, 75%, 80%, 85%, 90%, 95%, or 99% identical to any one of SEQ ID NOS: 39-40 (Table 2). In some embodiments, the immune checkpoint moiety comprises the extracellular domain of PD-L1. In some embodiments, the immune checkpoint moiety is the extracellular domain of PD-L1. In some embodiments, the immune checkpoint moiety comprises IGSF11 (VSIG-3) and is encoded from polynucleotide or a variation thereof or a fragment thereof that is at least 50%, 55%, 60%, 65%, 70%, 75%, 80%, 85%, 90%, 95%, or 99% identical to any one of SEQ ID NOS: 43-44 (Table 2). In some embodiments, the immune checkpoint moiety is CTLA-4 and encoded from polynucleotide or a variation thereof or a fragment thereof that is at least 50%, 55%, 60%, 65%, 70%, 75%, 80%, 85%, 90%, 95%, or 99% identical to any one of SEQ ID NOS: 41-42 (Table 2). In some embodiments, the immune checkpoint moiety comprises the extracellular domain of CTLA-4. In some embodiments, the immune checkpoint moiety is the extracellular domain of CTLA-4.

In some embodiments, the immune checkpoint moiety is encoded by a heterologous polynucleotide. In some embodiments, the heterologous polynucleotide is introduced into in any one of the cells described herein. In some embodiments, the heterologous polynucleotide comprises mRNA, rRNA, SRP RNA, tRNA, tmRNA, snRNA, snoRNA, gRNA, aRNA, crRNA, lncRNA, miRNA, ncRNA, piRNA, siRNA, and shRNA. In some cases, the heterologous polynucleotide comprises mRNA. In some embodiments, the heterologous polynucleotide comprises DNA. In some cases, the heterologous polynucleotide is inserted into the transmembrane moiety described herein. In some embodiments, the heterologous polynucleotide is inserted into the transmembrane moiety described herein via homologous recombination. In some embodiments, the heterologous polynucleotide comprises a nucleic sequence encoding VISTA, PD-L1, CTLA-4, PD-L2, B7-1 (CD80), B7-2 (CD86), B7-H3 (CD276), B7-H2, B7-H3, B7-H4 (VTCN1), IDO, KIR, LAG3, A2AR, HVEM (CD270, TNFRSF14), Galectin 9, Galectin3, CEACAM1 (CD66a), OX-2 (CD200), PVR (CD155), PVRL2 (Nectin-2, CD112), FGL-1, PECAM-1, TSG-6, CD47, Stabilin-1 (Clever-1), Neuropilin 1, Neuropilin 2, CD158 (family), IGSF2 (CD101), CD155, GITRL, CD137L, OX40L, LIGHT, CD70, PD-1, RGMB, CTLA-4 (CD152), BTLA, CD160, Tim-3, CD200R, TIGIT, CD112R (PVRIG), LAG-3 (CD223), PECAM-1, CD44, IGSF11 (VSIG-3), or SIRP alpha (CD172a). In some embodiments, the immune checkpoint moiety is encoded from a heterologous polynucleotide encoding VISTA. In some embodiments, the immune checkpoint moiety is encoded from a heterologous polynucleotide encoding PD-L1. In some embodiments, the immune checkpoint moiety is encoded from a heterologous polynucleotide encoding IGSF11 (VSIG-3). In some embodiments, the immune checkpoint moiety is encoded from a heterologous polynucleotide encoding CTLA-4.

In some embodiments, the immune checkpoint moiety comprises a heterologous polynucleotide encoding a cytokine. In some embodiments, the immune checkpoint moiety comprises a polypeptide comprising a peptide sequence of the cytokine. Exemplary cytokines that can be utilized as the immune check point moiety includes 4-1BBL, acylation stimulating protein, adipokine, albinterferon, APRIL, Arh, BAFF, Bcl-6, CCL1, CCL1/TCA3, CCL11, CCL12/MCP-5, CCL13/MCP-4, CCL14, CCL15, CCL16, CCL17/TARC, CCL18, CCL19, CCL2, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CCR10, CCR3, CCR4, CCR5, CCR6, CCR7, CCR8, CD153, CD154, CD178, CD40LG, CD70, CD95L/CD178, Cerberus (protein), chemokines, CLCF1, CNTF, colony-stimulating factor, common b chain (CD131), common g chain (CD132), CX3CL1, CX3CR1, CXCL1, CXCL10, CXCL11, CXCL12, CXCL13, CXCL14, CXCL15, CXCL16, CXCL17, CXCL2, CXCL2/MIP-2, CXCL3, CXCL4, CXCL5, CXCL6, CXCL7, CXCL9, CXCR3, CXCR4, CXCR5, EDA-A1, Epo, erythropoietin, FAM19A1, FAM19A2, FAM19A3, FAM19A4, FAM19A5, Flt-3L, FMS-like tyrosine kinase 3 ligand, Foxp3, GATA-3, GcMAF, G-CSF, GITRL, GM-CSF, granulocyte colony-stimulating factor, granulocyte-macrophage colony-stimulating factor, hepatocyte growth factor, IFNA1, IFNA10, IFNA13, IFNA14, IFNA2, IFNA4, IFNA5/IFNaG, IFNA7, IFNA8, IFNB1, IFNE, IFNG, IFNZ, IFN-α, IFN-β, IFN-γ, IFNω/IFNW1, IL-1, IL-10, IL-10 family, IL-10-like, IL-11, IL-12, IL-13, IL-14, IL-15, IL-16, IL-17, IL-17 family, IL-17A-F, IL-18, IL-18BP, IL-19, IL-1A, IL-1B, IL-1F10, IL-1F3/IL-1RA, IL-1F5, IL-1F6, IL-1F7, IL-1F8, IL-1F9, IL-1-like, IL-1RA, IL-1RL2, IL-1α, IL-1β, IL-2, IL-20, IL-21, IL-22, IL-23, IL-24, IL-28A, IL-28B, IL-29, IL-3, IL-31, IL-33, IL-35, IL-4, IL-5, IL-6, IL-6-like, IL-7, IL-8/CXCL8, IL-9, inflammasome, interferome, interferon, interferon beta-1a, interferon beta-1b, interferon gamma, interferon type I, interferon type II, interferon type III, interferons, interleukin, interleukin 1 receptor antagonist, Interleukin 8, IRF4, Leptin, leukemia inhibitory factor (LIF), leukocyte-promoting factor, LIGHT, LTA/TNFB, LT-β, lymphokine, lymphotoxin, lymphotoxin alpha, lymphotoxin beta, macrophage colony-stimulating factor, macrophage inflammatory protein, macrophage-activating factor, M-CSF, MHC class III, miscellaneous hematopoietins, monokine, MSP, myokine, myonectin, nicotinamide phosphoribosyltransferase, oncostatin M (OSM), oprelvekin, OX40L, platelet factor 4, promegapoietin, RANKL, SCF, STAT3, STAT4, STAT6, stromal cell-derived factor 1, TALL-1, TBX21, TGF-α, TGF-β, TGF-β1, TGF-β2, TGF-β3, TNF, TNFSF10, TNFSF11, TNFSF12, TNFSF13, TNFSF14, TNFSF15, TNFSF4, TNFSF8, TNF-α, TNF-β, Tpo, TRAIL, TRANCE, TWEAK, vascular endothelial growth inhibitor, XCL1, or XCL2.

In some embodiments, the immune checkpoint moiety is complexed with a transmembrane moiety described herein. In some embodiments, the immune checkpoint moiety is non-covalently complexed with the transmembrane moiety described herein. In some embodiments, the immune checkpoint moiety is covalently complexed with the transmembrane moiety described herein. In some embodiments, the immune check point moiety comprises the immune check point moiety conjugated with the transmembrane moiety via the affinity receiver/affinity tag system. In some embodiments, the immune check point moiety is coupled to the affinity receiver and the transmembrane moiety is coupled to the affinity tag, and the affinity tag is coupled to the affinity receiver. In some embodiments, the immune check point moiety is coupled to the affinity tag and the transmembrane moiety is coupled to the affinity receiver, and the affinity receiver is coupled to the affinity tag. In some embodiments, the immune checkpoint moiety is expressed as part of a fusion protein comprising both the immune checkpoint moiety and the transmembrane moiety. In some embodiments, the immune checkpoint moiety is expressed as part of a fusion protein comprising both the immune checkpoint moiety and a fragment of the transmembrane moiety. In some embodiments, the N-terminus of the immune checkpoint moiety is fused to the transmembrane moiety. In some embodiments, the C-terminus of the immune checkpoint moiety is fused to the transmembrane moiety described herein. In some embodiments, the immune checkpoint moiety is fused and flanked by the transmembrane moiety on both N and C-terminus of the immune checkpoint moiety. For example, the immune checkpoint moiety is inserted into a transmembrane moiety as part of a fusion peptide, where the N-terminus of the fusion peptide comprises a fragment of the transmembrane moiety, followed by the immune checkpoint moiety (or a variation there or a fragment thereof), and followed by the C-terminus of the fusion peptide comprising another fragment of the transmembrane moiety. In some embodiments, the immune checkpoint moiety comprises the immune checkpoint moiety complexed with the transmembrane moiety. In some embodiments, the immune checkpoint moiety comprises the immune checkpoint moiety non-covalently complexed with the transmembrane moiety. In some embodiments, the immune checkpoint moiety comprises the immune checkpoint moiety covalently complexed with the transmembrane moiety. In some embodiments, the immune check point moiety comprises the immune check point moiety conjugated with the transmembrane moiety via the affinity receiver/affinity tag system. In some embodiments, the immune check point moiety is coupled to the affinity receiver and the transmembrane moiety is coupled to the affinity tag, and the affinity tag is coupled to the affinity receiver. In some embodiments, the immune check point moiety is coupled to the affinity tag and the transmembrane moiety is coupled to the affinity receiver, and the affinity receiver is coupled to the affinity tag.

In some embodiments, the engineered extracellular vesicle comprises a plurality of the immune checkpoint moiety described herein. In some embodiments, the plurality of the immune checkpoint moieties is expressed on the surface of the engineered extracellular vesicle. In some embodiments, the plurality of the immune checkpoint moieties is expressed on the surface of the engineered extracellular vesicle as part of the fusion with the transmembrane moiety. In some embodiments, the engineered extracellular vesicle expresses at least one, ten, 100, 500, 1,000, 5,000, 10,000, 50,000, 100,000, 500,000, 1,000,000, 5,000,000, or more units of the immune checkpoint moiety. In some embodiments, the engineered extracellular vesicle delivers the expressed immune checkpoint moiety to a target cell or a target microenvironment.

In some embodiments, the engineered extracellular vesicle comprises a plurality of the immune checkpoint moiety described herein. In some embodiments, the plurality of the immune checkpoint moieties is encapsulated in the engineered extracellular vesicle. In some embodiments, the engineered extracellular vesicle encapsulates at least one, ten, 100, 500, 1,000, 5,000, 10,000, 50,000, 100,000, 500,000, 1,000,000, 5,000,000, or more units of the immune checkpoint moiety. In some embodiments, the engineered extracellular vesicle delivers the encapsulated immune checkpoint moiety to a target cell or a target microenvironment.

In some embodiments, the engineered extracellular vesicle secretes or releases a plurality of the immune checkpoint moiety described herein. In some embodiments, the engineered extracellular vesicle secretes at least one, ten, 100, 500, 1,000, 5,000, 10,000, 50,000, 100,000, 500,000, 1,000,000, 5,000,000, or more units of the immune checkpoint moiety. In some embodiments, the engineered extracellular vesicle secretes the immune checkpoint moiety to a target cell or a target environment.

In some embodiments, the plurality of the immune checkpoint moiety is expressed on the surface of the engineered extracellular vesicle. In some embodiments, the plurality of the immune checkpoint moieties is expressed as part of the fusion peptide comprising immune checkpoint moiety and transmembrane moiety. In some instances, the engineered extracellular vesicle comprising the immune checkpoint moiety expressed on the surface of the engineered extracellular vesicle contacts with a target cell or a target environment.

In some embodiments, the number of units of the immune checkpoint moiety that is expressed on the surface of the engineered extracellular vesicle is limited by a theoretical maximum as determined by the ratio between: the dimensions of the engineered extracellular vesicle; and the dimensions of the expressed immune checkpoint moiety or the expressed fusion peptide comprising the immune checkpoint moiety. In some embodiments, the platforms and methods described herein can generate and select for an extracellular vesicle expressing a number of units of immune checkpoint moiety that is at least 30%, 35%, 40%, 45%, 50%, 55%, 60%, 65%, 70%, 75%, 80%, 85%, 90%, 95%, or 99% of the theoretical maximum of number of units of immune checkpoint moiety that can expressed on the surface of the engineered extracellular vesicle.

### Fusion Proteins

Described herein, in some embodiments, is a composition comprising a fusion protein or polypeptide, with the fusion protein or polypeptide comprising an immune checkpoint moiety and a transmembrane moiety.

In some embodiments, the fusion peptide or polypeptide comprises a plurality of immune checkpoint moieties. In some embodiments, the fusion protein or polypeptide comprises both an immune checkpoint and a transmembrane moiety comprising a full-length protein, a variation thereof, or a fragment thereof. In some embodiments, the fusion peptide of polypeptide comprises a plurality of transmembrane moieties. In some embodiments, there is a plurality of immune checkpoint moieties. In some embodiments, there is a single immune checkpoint moiety.

In some embodiments, the immune checkpoint moiety is endogenous to the cell that is generating the engineered extracellular vesicles. In some embodiments, the immune checkpoint moiety is heterologous to the cell that is generating the engineered extracellular vesicles. In some embodiments, the immune checkpoint moiety comprises therapeutic properties for treating diseases or disorders. In some embodiments, the immune checkpoint moiety comprises therapeutic properties for treating an inflammatory or autoimmune disease or disorder described herein. In some embodiments, the immune checkpoint moiety targets and modulates activities of immune cells. In some embodiments, the immune cells comprises T cell, including cytotoxic T cell, Natural Killer T cell, Regulatory T cell, and T helper cells. In some embodiments, the immune cells comprises CD8+ cells. In some embodiments, the immune cells comprises CD25+ cells. In some embodiments, the immune cells comprises CD4+ cells. In some embodiments, the immune cells comprises CD8+CD25+ cells. In some cases, the immune cell comprises cell that expresses CD4. In some cases, the immune cell comprises cell that expresses CD4 and CD 25 (CD4+CD25+). In some cases, the immune cell comprises cell that expresses FOXP3. In some cases, the immune cell comprises cell that expresses CD4, CD25, and FOXP3 (CD4+CD25+FOXP3+).

In some embodiments, the one or more immune checkpoint moieties comprises the extracellular domain of the respective immune checkpoint moieties. In some embodiments, the one or more immune checkpoint moieties are the extracellular domain of the respective immune checkpoint moieties. For example, in some embodiments, the one or more immune checkpoint moieties comprises an extracellular domain of PD-L1 and an extracellular domain of CD200. In some embodiments, the extracellular domain of the immune checkpoint moiety is linked (e.g., directly linked) to an affinity tag. In some embodiments, the one or more immune checkpoints comprise a first extracellular domain of a first immune checkpoint moiety that is linked (e.g., directly linked) to a first affinity tag, and a second extracellular domain of a second immune checkpoint moiety that is linked (e.g., directly linked) to a second affinity tag.

In some embodiments, the one or more immune checkpoint moieties comprises VISTA, CTLA-4, PD-L1, PD-1, IGSF11 (VSIG-3), or a combination thereof. In some embodiments, the immune checkpoint moiety comprises a polypeptide sequence that is at least about 60%, about 65%, about 70%, about 75%, about 80%, about 85%, about 90%, about 95%, about 96%, about 97%, about 98%, about 99%, or from 60-100%, 60-90%, 60-80%, 60-70%, 70-100%, 70-90%, 70-80%, 80-100%, 80-90%, 90-100% identical to VISTA, PD-L1, CTLA-4, PD-L2, B7-1 (CD80), B7-2 (CD86), B7-H3 (CD276), B7-H2, B7-H4 (VTCN1), HVEM (CD270, TNFRSF14), Galectin 9, Galectin3, CEACAM1 (CD66a), OX-2 (CD200), PVR (CD155), PVRL2 (Nectin-2, CD112), FGL-1, PECAM-1, TSG-6, CD47, Stabilin-1 (Clever-1), Neuropilin 1, Neuropilin 2, CD158 (family), IGSF2 (CD101), CD155, GITRL, CD137L, OX40L, LIGHT, CD70, PD-1, RGMB, CTLA-4 (CD152), BTLA, CD160, Tim-3, CD200R, TIGIT, CD112R (PVRIG), LAG-3 (CD223), PECAM-1, CD44, SIRP alpha (CD172a), IGSF11 (VSIG-3), or a combination thereof. In some embodiments, the one or more immune checkpoint moieties comprises the extracellular domain of VISTA, PD-L1, CTLA-4, PD-L2, B7-1 (CD80), B7-2 (CD86), B7-H3 (CD276), B7-H2, B7-H4 (VTCN1), HVEM (CD270, TNFRSF14), Galectin 9, Galectin3, CEACAM1 (CD66a), OX-2 (CD200), PVR (CD155), PVRL2 (Nectin-2, CD112), FGL-1, PECAM-1, TSG-6, CD47, Stabilin-1 (Clever-1), Neuropilin 1, Neuropilin 2, CD158 (family), IGSF2 (CD101), CD155, GITRL, CD137L, OX40L, LIGHT, CD70, PD-1, RGMB, CTLA-4 (CD152), BTLA, CD160, Tim-3, CD200R, TIGIT, CD112R (PVRIG), LAG-3 (CD223), PECAM-1, CD44, SIRP alpha (CD172a), IGSF11 (VSIG-3), or a combination thereof. In some embodiments, the one or more immune checkpoint moieties is the extracellular domain of VISTA, PD-L1, CTLA-4, PD-L2, B7-1 (CD80), B7-2 (CD86), B7-H3 (CD276), B7-H2, B7-H4 (VTCN1), HVEM (CD270, TNFRSF14), Galectin 9, Galectin3, CEACAM1 (CD66a), OX-2 (CD200), PVR (CD155), PVRL2 (Nectin-2, CD112), FGL-1, PECAM-1, TSG-6, CD47, Stabilin-1 (Clever-1), Neuropilin 1, Neuropilin 2, CD158 (family), IGSF2 (CD101), CD155, GITRL, CD137L, OX40L, LIGHT, CD70, PD-1, RGMB, CTLA-4 (CD152), BTLA, CD160, Tim-3, CD200R, TIGIT, CD112R (PVRIG), LAG-3 (CD223), PECAM-1, CD44, SIRP alpha (CD172a), IGSF11 (VSIG-3), or a combination thereof. In some embodiments, the immune checkpoint moiety is encoded by a polynucleotide sequence encoding VISTA, PD-L1, IGSF11 (VSIG-3), or CLLA-4. In some embodiments, the immune checkpoint moiety comprises a polynucleotide sequence encoding a polypeptide or a variation thereof or a fragment thereof. In some embodiments, the immune checkpoint moiety comprises a polypeptide or a variation thereof or a fragment thereof. In some embodiments, the immune checkpoint moiety is encoded by a polynucleotide sequence that is at least 50%, 55%, 60%, 65%, 70%, 75%, 80%, 85%, 90%, 95%, or 99% identical to any one of SEQ ID NOS: 37-44 (Table 2).

In some embodiments, the immune checkpoint moiety is encoded by a polynucleotide sequence that is at least 50%, 55%, 60%, 65%, 70%, 75%, 80%, 85%, 90%, 95%, or 99% identical to SEQ ID NO: 37. In some embodiments, the immune checkpoint moiety is encoded by a polynucleotide sequence that is at least 50%, 55%, 60%, 65%, 70%, 75%, 80%, 85%, 90%, 95%, or 99% identical to SEQ ID NO: 38. In some embodiments, the immune checkpoint moiety is encoded by a polynucleotide sequence that is at least 50%, 55%, 60%, 65%, 70%, 75%, 80%, 85%, 90%, 95%, or 99% identical to SEQ ID NO: 39. In some embodiments, the immune checkpoint moiety is encoded by a polynucleotide sequence that is at least 50%, 55%, 60%, 65%, 70%, 75%, 80%, 85%, 90%, 95%, or 99% identical to SEQ ID NO: 40. In some embodiments, the immune checkpoint moiety is encoded by a polynucleotide sequence that is at least 50%, 55%, 60%, 65%, 70%, 75%, 80%, 85%, 90%, 95%, or 99% identical to SEQ ID NO: 41. In some embodiments, the immune checkpoint moiety is encoded by a polynucleotide sequence that is at least 50%, 55%, 60%, 65%, 70%, 75%, 80%, 85%, 90%, 95%, or 99% identical to SEQ ID NO: 42. In some embodiments, the immune checkpoint moiety is encoded by a polynucleotide sequence that is at least 50%, 55%, 60%, 65%, 70%, 75%, 80%, 85%, 90%, 95%, or 99% identical to SEQ ID NO: 43. In some embodiments, the immune checkpoint moiety is encoded by a polynucleotide sequence that is at least 50%, 55%, 60%, 65%, 70%, 75%, 80%, 85%, 90%, 95%, or 99% identical to SEQ ID NO: 44.

In some embodiments, the immune checkpoint moiety comprises VISTA and is encoded from polynucleotide or a variation thereof or a fragment thereof that is at least 50%, 55%, 60%, 65%, 70%, 75%, 80%, 85%, 90%, 95%, or 99% identical to any one of SEQ ID NOS: 37-38. In some embodiments, the immune checkpoint moiety comprises PD-L1 and is encoded from polynucleotide or a variation thereof or a fragment thereof that is at least 50%, 55%, 60%, 65%, 70%, 75%, 80%, 85%, 90%, 95%, or 99% identical to any one of SEQ ID NOS: 39-40. In some embodiments, the immune checkpoint moiety comprises IGSF11 (VSIG-3) and is encoded from polynucleotide or a variation thereof or a fragment thereof that is at least 50%, 55%, 60%, 65%, 70%, 75%, 80%, 85%, 90%, 95%, or 99% identical to any one of SEQ ID NOS: 43-44. In some embodiments, the immune checkpoint moiety comprises CTLA-4 and is encoded from polynucleotide or a variation thereof or a fragment thereof that is at least 50%, 55%, 60%, 65%, 70%, 75%, 80%, 85%, 90%, 95%, or 99% identical to any one of SEQ ID NOS: 41-42.

In some embodiments, the immune checkpoint moiety is encoded by a heterologous polynucleotide. In some embodiments, the heterologous polynucleotide is introduced into in any one of the cells described herein. In some embodiments, the heterologous polynucleotide comprises mRNA, rRNA, SRP RNA, tRNA, tmRNA, snRNA, snoRNA, gRNA, aRNA, crRNA, lncRNA, miRNA, ncRNA, piRNA, siRNA, and shRNA. In some cases, the heterologous polynucleotide comprises mRNA. In some embodiments, the heterologous polynucleotide comprises DNA. In some cases, the heterologous polynucleotide is inserted into the transmembrane moiety described herein. In some embodiments, the heterologous polynucleotide is inserted into the transmembrane moiety described herein via homologous recombination. In some embodiments, the heterologous polynucleotide comprises a nucleic sequence encoding VISTA, PD-L1, CTLA-4, PD-L2, B7-1 (CD80), B7-2 (CD86), B7-H3 (CD276), B7-H2, B7-H3, B7-H4 (VTCN1), IDO, KIR, LAG3, A2AR, HVEM (CD270, TNFRSF14), Galectin 9, Galectin3, CEACAM1 (CD66a), OX-2 (CD200), PVR (CD155), PVRL2 (Nectin-2, CD112), FGL-1, PECAM-1, TSG-6, CD47, Stabilin-1 (Clever-1), Neuropilin 1, Neuropilin 2, CD158 (family), IGSF2 (CD101), CD155, GITRL, CD137L, OX40L, LIGHT, CD70, PD-1, RGMB, CTLA-4 (CD152), BTLA, CD160, Tim-3, CD200R, TIGIT, CD112R (PVRIG), LAG-3 (CD223), PECAM-1, CD44, IGSF11 (VSIG-3), or SIRP alpha (CD172a). In some embodiments, the immune checkpoint moiety is encoded from a heterologous polynucleotide encoding VISTA. In some embodiments, the immune checkpoint moiety is encoded from a heterologous polynucleotide encoding PD-L1. In some embodiments, the immune checkpoint moiety is encoded from a heterologous polynucleotide encoding IGSF11 (VSIG-3). In some embodiments, the immune checkpoint moiety is encoded from a heterologous polynucleotide encoding CTLA-4.

In some embodiments, the immune checkpoint moiety comprises a heterologous polynucleotide encoding a cytokine. In some embodiments, the immune checkpoint moiety comprises a polypeptide comprising a peptide sequence of the cytokine. Exemplary cytokines that can be utilized as the immune check point moiety includes 4-1BBL, acylation stimulating protein, adipokine, albinterferon, APRIL, Arh, BAFF, Bcl-6, CCL1, CCL1/TCA3, CCL11, CCL12/MCP-5, CCL13/MCP-4, CCL14, CCL15, CCL16, CCL17/TARC, CCL18, CCL19, CCL2, CCL2/MCP-1, CCL20, CCL21, CCL22/MDC, CCL23, CCL24, CCL25, CCL26, CCL27, CCL28, CCL3, CCL3L3, CCL4, CCL4L1/LAG-1, CCL5, CCL6, CCL7, CCL8, CCL9, CCR10, CCR3, CCR4, CCR5, CCR6, CCR7, CCR8, CD153, CD154, CD178, CD40LG, CD70, CD95L/CD178, Cerberus (protein), chemokines, CLCF1, CNTF, colony-stimulating factor, common b chain (CD131), common g chain (CD132), CX3CL1, CX3CR1, CXCL1, CXCL10, CXCL11, CXCL12, CXCL13, CXCL14, CXCL15, CXCL16, CXCL17, CXCL2, CXCL2/MIP-2, CXCL3, CXCL4, CXCL5, CXCL6, CXCL7, CXCL9, CXCR3, CXCR4, CXCR5, EDA-A1, Epo, erythropoietin, FAM19A1, FAM19A2, FAM19A3, FAM19A4, FAM19A5, Flt-3L, FMS-like tyrosine kinase 3 ligand, Foxp3, GATA-3, GcMAF, G-CSF, GITRL, GM-CSF, granulocyte colony-stimulating factor, granulocyte-macrophage colony-stimulating factor, hepatocyte growth factor, IFNA1, IFNA10, IFNA13, IFNA14, IFNA2, IFNA4, IFNA5/IFNaG, IFNA7, IFNA8, IFNB1, IFNE, IFNG, IFNZ, IFN-α, IFN-β, IFN-γ, IFNω/IFNW1, IL-1, IL-10, IL-10 family, IL-10-like, IL-11, IL-12, IL-13, IL-14, IL-15, IL-16, IL-17, IL-17 family, IL-17A-F, IL-18, IL-18BP, IL-19, IL-1A, IL-1B, IL-1F10, IL-1F3/IL-1RA, IL-1F5, IL-1F6, IL-1F7, IL-1F8, IL-1F9, IL-1-like, IL-1RA, IL-1RL2, IL-1α, IL-1β, IL-2, IL-20, IL-21, IL-22, IL-23, IL-24, IL-28A, IL-28B, IL-29, IL-3, IL-31, IL-33, IL-35, IL-4, IL-5, IL-6, IL-6-like, IL-7, IL-8/CXCL8, IL-9, inflammasome, interferome, interferon, interferon beta-1a, interferon beta-1b, interferon gamma, interferon type I, interferon type II, interferon type III, interferons, interleukin, interleukin 1 receptor antagonist, Interleukin 8, IRF4, Leptin, leukemia inhibitory factor (LIF), leukocyte-promoting factor, LIGHT, LTA/TNFB, LT-β, lymphokine, lymphotoxin, lymphotoxin alpha, lymphotoxin beta, macrophage colony-stimulating factor, macrophage inflammatory protein, macrophage-activating factor, M-CSF, MHC class III, miscellaneous hematopoietins, monokine, MSP, myokine, myonectin, nicotinamide phosphoribosyltransferase, oncostatin M (OSM), oprelvekin, OX40L, platelet factor 4, promegapoietin, RANKL, SCF, STAT3, STAT4, STAT6, stromal cell-derived factor 1, TALL-1, TBX21, TGF-α, TGF-β, TGF-β1, TGF-β2, TGF-β3, TNF, TNFSF10, TNFSF11, TNFSF12, TNFSF13, TNFSF14, TNFSF15, TNFSF4, TNFSF8, TNF-α, TNF-β, Tpo, TRAIL, TRANCE, TWEAK, vascular endothelial growth inhibitor, XCL1, or XCL2.

In some embodiments, the immune checkpoint moiety is complexed with the transmembrane moiety described herein to form the fusion proteins or polypeptides described herein. In some embodiments, the immune checkpoint moiety is non-covalently complexed with the transmembrane moiety described herein. In some embodiments, the immune checkpoint moiety is covalently complexed with the transmembrane moiety described herein. In some embodiments, the immune check point moiety comprises the immune check point moiety conjugated with the transmembrane moiety via the affinity receiver/affinity tag system. In some embodiments, the immune check point moiety is coupled to the affinity receiver and the transmembrane moiety is coupled to the affinity tag, and the affinity tag is coupled to the affinity receiver. In some embodiments, the immune check point moiety is coupled to the affinity tag and the transmembrane moiety is coupled to the affinity receiver, and the affinity receiver is coupled to the affinity tag. In some embodiments, the immune checkpoint moiety is expressed as part of a fusion protein comprising both the immune checkpoint moiety and the transmembrane moiety. In some embodiments, the immune checkpoint moiety is expressed as part of a fusion protein comprising both the immune checkpoint moiety and a fragment of the transmembrane moiety. In some embodiments, the N-terminus of the immune checkpoint moiety is fused to the transmembrane moiety. In some embodiments, the C-terminus of the immune checkpoint moiety is fused to the transmembrane moiety described herein. In some embodiments, the immune checkpoint moiety is fused and flanked by the transmembrane moiety on both N and C-terminus of the immune checkpoint moiety. For example, the immune checkpoint moiety is inserted into a transmembrane moiety as part of a fusion peptide, where the N-terminus of the fusion peptide comprises a fragment of the transmembrane moiety, followed by the immune checkpoint moiety (or a variation there or a fragment thereof), and followed by the C-terminus of the fusion peptide comprising another fragment of the transmembrane moiety. In some embodiments, the immune checkpoint moiety comprises the fusion peptide, where the immune checkpoint moiety is fused to the transmembrane moiety. In some embodiments, the immune checkpoint moiety comprises the immune checkpoint moiety complexed with the transmembrane moiety. In some embodiments, the immune checkpoint moiety comprises the immune checkpoint moiety non-covalently complexed with the transmembrane moiety. In some embodiments, the immune checkpoint moiety comprises the immune checkpoint moiety covalently complexed with the transmembrane moiety. In some embodiments, the immune check point moiety comprises the immune check point moiety conjugated with the transmembrane moiety via the affinity receiver/affinity tag system. In some embodiments, the immune check point moiety is coupled to the affinity receiver and the transmembrane moiety is coupled to the affinity tag, and the affinity tag is coupled to the affinity receiver. In some embodiments, the immune check point moiety is coupled to the affinity tag and the transmembrane moiety is coupled to the affinity receiver, and the affinity receiver is coupled to the affinity tag.

In some embodiments, a plurality of immune checkpoint moieties are expressed on the surface of an extracellular vesicle. In some embodiments, the extracellular vesicle is an engineered extracellular vesicle. In some embodiments, the number of units of the immune checkpoint moiety that can be expressed on the surface of the engineered extracellular vesicle is limited by a theoretical maximum as determined by the ratio between: the dimensions of the engineered extracellular vesicle; and the dimensions of the expressed immune checkpoint moiety or the expressed fusion peptide comprising the immune checkpoint moiety

In some embodiments, the composition fusion protein or polypeptide is expressed on the surface of an extracellular vesicle.

In some embodiments, the fusion protein or polypeptide is encoded for by a heterologous polynucleotide.

In some embodiments, the fusion protein or polypeptide comprise at least one transmembrane moiety. In some embodiments, the transmembrane moiety comprises a full-length protein or a variation thereof or a fragment thereof. In some embodiments, the transmembrane moiety is endogenous to the cell that is generating the engineered extracellular vesicles. In some embodiments, the immune checkpoint moiety is heterologous to the cell that is generating the engineered extracellular vesicles. In some embodiments, the transmembrane moiety is selected from a group consisting of: 14-3-3 protein zeta/delta, 14-3-3 protein epsilon, 78 kDa glucose-regulated protein, acetylcholinesterase/AChE-S, AChE-E, actin, cytoplasmic 1 (ACTA), ADAM10, alkaline phosphatase, alpha-enolase, alpha-synuclein, aminopeptidase N, amyloid beta A4/APP, annexin 5A, annexin A2, AP-1, ATF3, ATP citrate lyase, ATPase, beta actin (ACTB), beta-amyloid 42, caveolin-1, CD10, CD11a, CD11b, CD11c, CD14, CD142, CD146, CD163, CD24, CD26/DPP4, CD29/ITGB1, CD3, CD37, CD41, CD42a, CD44, CD45, CD47, CD49, CD49d, CD53, CD63, CD64, CD69, CD73 CD81, CD82, CD9, CD90, claudin, claudin-1 cofilin-1, complement-binding proteins CD55 and CD59, cytosolic heat shock protein 90 alpha, cytosolic heat shock protein 90 beta, EBV LMP1, EBV LMP2A, EF-1alpha-1, EF2, EFGR EGFR VIII, emmprin/CD147, enolase 1 alpha (ENO1), EPCAM, ERBB2, tetraspanins (CD9, CD63 and CD81), fatty acid synthase, fetuin-A, flotillin-1, flotillin-2, fructose-bisphosphate aldolase A, glyceraldehyde-3-phosphate dehydrogenase (GAPDH), glycophorin A, GPC1, GPI-anchored 5′nucleotidase, GTPase, heat shock protein 8 (HSPA8), heat shock proteins (HSP70 and HSP90), heparan sulfate proteoglycans, heparinase, heterotrimeric G proteins, HIV Gag, HIV Nef, HLA-DRA, HLA-G, HSV gB, HTLV-1 Tax, huntingtin, ICAM1, integrins, lactadherin, LAMP1/2, LAMP2A, LAMP2B, LAMP2C, leucine-rich receptor kinase 2, L-lactate dehydrogenase A chain, lysosome-associated membrane glycoprotein 1, lysosome-associated membrane glycoprotein 2, MHC class I, MHC class II, MUC1, multidrug resistance-associated protein, muscle pyruvate kinase (PKM2), N-cadherin, NKCC2, PDCD6IP/Alix, PECAM1, phosphoglycerate kinase, placental prion proteins, prostate-specific antigen (PSA), pyruvate kinase (PKM), Rab-14, Rab-5a, Rab-5b, Rab-5c, Rab-7, Rap 1B, resistin, sonic hedgehog (SHH), surviving, syndecan-1, syndecan-4, syntenin-1, transferrin receptor (TFR2), TSG101, TSPAN8, tumor-associated glycoprotein tetraspanin-8, tyrosine 3 monooxygenase/tryptophan 5-monooxygenase activation protein, TYRP-2, or vacuolar-sorting-associated protein 35 (VPS35)

In some embodiments, the transmembrane moiety comprises LAMP2. In some embodiments, the transmembrane moiety comprises LAMP-like domain 1 of LAMP2. In some embodiments, the transmembrane moiety comprises LAMP2B. In some embodiments, the transmembrane moiety comprises a polypeptide sequence that is at least 50%, 55%, 60%, 65%, 70%, 75%, 80%, 85%, 90%, 95%, or 99% identical to SEQ ID NO: 1 (Table 1). In some embodiments, the transmembrane moiety is encoded from a polynucleotide sequence comprising homology arms. In some instances, the immune checkpoint moiety described herein is inserted into the transmembrane moiety by homologous recombination as induced by the homology arms of the transmembrane moiety. In some embodiments, the transmembrane moiety comprises an N-terminus homology arm or a C-terminus homology arm. In some embodiments, the N-terminus homology of the transmembrane moiety is encoded from a polynucleotide sequence that is at least 50%, 55%, 60%, 65%, 70%, 75%, 80%, 85%, 90%, 95%, or 99% identical to SEQ ID NO: 2 (Table 1). In some embodiments, the C-terminus homology of the transmembrane moiety is encoded from a polynucleotide sequence that is at least 50%, 55%, 60%, 65%, 70%, 75%, 80%, 85%, 90%, 95%, or 99% identical to SEQ ID NO: 3 (Table 1).

In some embodiments, the transmembrane moiety is complexed with the immune checkpoint moiety described herein to generate the fusion proteins or polypeptides described herein. In some embodiments, the transmembrane moiety is non-covalently complex with the immune checkpoint moiety described herein. In some embodiments, the transmembrane moiety is covalently complexed with the immune checkpoint moiety described herein. In some embodiments, the transmembrane moiety is fused to the immune checkpoint moiety described herein at the N-terminus of the transmembrane moiety. In some embodiments, the transmembrane moiety is fused to the immune checkpoint moiety described herein at the C-terminus of the transmembrane moiety. In some embodiments, the immune checkpoint moiety is inserted (e.g. via homology recombination) at any locus of the transmembrane moiety.

In some embodiments, the transmembrane moiety is covalently connected to the immune checkpoint moiety by a peptidyl linker. In some cases, the linker is a flexible linker, a rigid linker, or a cleavable linker. In some embodiments, the flexible linker is (GGGGS)n where n=1, 2, 3, or 4; KESGSVSSEQLAQFRSLD (SEQ ID NO: 59); EGKSSGSGSESKST (SEQ ID NO: 60); GGGGGG (SEQ ID NO: 61); GGGGGGGG (SEQ ID NO: 62); GSAGSAAGSGEF (SEQ ID NO: 63); RKRR (SEQ ID NO: 64); SS (SEQ ID NO: 65); or LE (SEQ ID NO: 66). In some instances, the rigid linker is (EAAAK)n where n=1, 2, 3, or 4; A(EAAAK)nA where n=2, 3, 4, or 5; A(EAAAK)4ALEA(EAAAK)4A (SEQ ID NO: 68); AEAAAKEAAAKA (SEQ ID NO: 99); or LEAGCKNFFPRSFTSCGSLE (SEQ ID NO: 70). In some aspects, the cleavable linker is LEAGCKNFFPRSFTSCGSLE (SEQ ID NO: 71); CRRRRRREAEAC (SEQ ID NO: 72); GGIEGRGS (SEQ ID NO: 73); or TRHRQPRGWEQL (SEQ ID NO: 74).

### Growth Factor

In some embodiments, the composition described herein comprises one or more growth factors or fragments thereof. Examples of the growth factor include, without limitation, EGF, FGF, NGF, PDGF, VEGF, IGF, GMCSF, GCSF, TGF, BMP, HGF, GDF, MSF, SGF, GDF, brain-derived neurotrophic factor (BNDF), glial cell-derived neurotrophic factor (GDNF), neurotrophin nerve growth factor (NGF), neurotrophin-3, neurotrophin-4, ciliary neurotrophic factor, artemin, neurturin, IL-2, and TGF-Beta.

In some embodiments, the growth factor is conjugated with the transmembrane moiety described herein. In some embodiments, the growth factor is non-covalently conjugated with the transmembrane moiety described herein. In some embodiments, the growth factor is covalently conjugated with the transmembrane moiety described herein. In some embodiments, the growth factor is conjugated with the transmembrane moiety via the affinity receiver/affinity tag system. In some embodiments, the growth factor is expressed as part of a fusion protein comprising both the growth factor and the transmembrane moiety. In some embodiments, the growth factor is expressed as part of a fusion protein comprising both the growth factor and a fragment of the transmembrane moiety. In some embodiments, the N-terminus of the growth factor is fused to the transmembrane moiety. In some embodiments, the C-terminus of the growth factor is fused to the transmembrane moiety described herein. In some embodiments, the growth factor is fused and flanked by the transmembrane moiety on both N and C-terminus of the growth factor. For example, the growth factor is inserted into a transmembrane moiety as part of a fusion peptide, where the N-terminus of the fusion peptide comprises a fragment of the transmembrane moiety, followed by the growth factor (or a variation thereof or a fragment thereof), and followed by the C-terminus of the fusion peptide comprising another fragment of the transmembrane moiety. In some embodiments, the growth factor comprises the growth factor conjugated with the transmembrane moiety. In some embodiments, the growth factor comprises the growth factor non-covalently conjugated with the transmembrane moiety. In some embodiments, the growth factor comprises the growth factor covalently conjugated with the transmembrane moiety. In some embodiments, the growth factor comprises the growth factor conjugated with the transmembrane moiety via the affinity receiver/affinity tag system. In some embodiments, the growth factor is coupled to the affinity receiver and the transmembrane moiety is coupled to the affinity tag, and the affinity tag is coupled to the affinity receiver. In some embodiments, the growth factor is coupled to the affinity tag and the transmembrane moiety is coupled to the affinity receiver, and the affinity receiver is coupled to the affinity tag.

In some embodiments, the engineered extracellular vesicle described herein comprises a plurality of the growth factors. In some embodiments, the plurality of the growth factors is expressed on the surface of the engineered extracellular vesicle. In some embodiments, the plurality of the growth factors is expressed on the surface of the engineered extracellular vesicle as part of the fusion with the transmembrane moiety. In some embodiments, the engineered extracellular vesicle expresses at least one, ten, 100, 500, 1,000, 5,000, 10,000, 50,000, 100,000, 500,000, 1,000,000, 5,000,000, or more units of the growth factor. In some embodiments, the engineered extracellular vesicle delivers the expressed growth factor to a target cell or a target microenvironment.

### Affinity Receiver/Affinity Tag System

In some embodiments, the fusion peptide comprises any one of the transmembrane moieties or fragments thereof described herein and any one of the affinity receivers described herein. In some embodiments, the affinity receiver described herein recognize and reacts with the affinity tag to create a multiple-protein complex. Upon recognition, the affinity tag binds to the affinity receiver to form an intermolecular bond. In some embodiments, the affinity receiver and the affinity tag form a reversible noncovalent bond. In some embodiments, the affinity receiver and the affinity tag form an irreversible covalent bond. In some embodiments, the affinity receiver and the affinity tag form an isopeptide bond. In some embodiments, the affinity receiver and the affinity tag form a disulfide bond. This affinity receiver/affinity tag system allows ligation of two molecules (e.g., protein or peptide) that would otherwise be difficult or impossible to conjugate with traditional direct gene fusion methods.

In some embodiments, the protein, peptide, or molecule to be loaded (i.e., “passenger” protein, peptide, or molecule) is fused with an affinity tag that can bind to the affinity receiver on the anchor protein on the surface of an exosome. When a passenger protein having the affinity tag is mixed with an exosome carrying the anchor protein having the affinity receiver, the affinity tag binds the affinity receiver, bringing the passenger protein to the surface of the exosome. This loading approach using an affinity receiver-affinity tag system is superior to traditional methods because some passenger proteins cannot be directly loaded onto exosomes using traditional methods, for example, signaling proteins, immune checkpoint molecules, secreted growth factors and large proteins.

In some embodiments, the affinity receiver is a protein with a reactive Lys. The affinity tag is a 13-residue peptide with a reactive Asp. Upon mixing, an isopeptide bond is formed between the side chains of Lys in the affinity receiver and Asp in the affinity tag. In some embodiments, the affinity receiver and the affinity tag is genetically fused to the N- or C-terminus of proteins, and in some cases within internal loops of proteins. In preferred embodiments, neither the affinity receiver nor the affinity tag contains cysteine.

In various embodiments comprising the fusion peptide comprising the affinity receiver, an affinity tag is conjugated to any one of the immune checkpoint moieties or growth factors disclosed herein. In some embodiments, the affinity receiver and affinity tag are conjugated to form a complex of the transmembrane moiety and the immunity checkpoint moiety or growth factor wherein the transmembrane moiety and the immune checkpoint moiety or growth factor are coupled together via the affinity receiver and the affinity tag.

In preferred embodiments, the affinity receiver comprises a sequence that is at least 10%, 20%, 30%, 40%, 50%, 60%, 70%, 75%, 80%, 85%, 90%, 95%, 99% or 100% homologous to SEQ ID NO: 20. In preferred embodiments, the affinity receiver comprises a sequence that is at least 10%, 20%, 30%, 40%, 50%, 60%, 70%, 75%, 80%, 85%, 90%, 95%, 99% or 100% homologous to SEQ ID NO. 21.

In some embodiments, the affinity tag comprises a peptide having an amino acid sequence that is configured to bind to a portion of the affinity receiver. In some embodiments, the affinity tag binds to an affinity receiver through a covalent bond. In some embodiments, the affinity tag binds to an affinity receiver through an iso-peptide bond. In some embodiments, the affinity tag binds to an affinity receiver through a di-sulfide bond. In some embodiments, the affinity tag binds to an affinity receiver through electrical charges between one or more amino acids having positively charged side chains and one or more amino acids having negatively charged side chains.

TABLE 4
Exemplary affinity receiver sequences

| | |
|---|---|
| | Affinity receiver |
| SEQ ID NO: 20 | MVTTLSGLSGEQGPSGDMTTEEDSATHIKFSKRD |
| | EDGRELAGATMELRDSSGKTISTWISDGHVKDFY |
| | LYPGKYTFVETAAPDGYEVATPIEFTVNEDGQVT |
| | VDGEATEGDAHTGSSGS |
| SEQ ID NO: 21 | atggtaaccaccttatcaggtttatcaggtgagc |
| | aaggtccgtccggtgatatgacaactgaagaaga |
| | tagtgctacccatattaaattctcaaaacgtgat |
| | gaggacggccgtgagttagctggtgcaactatgg |
| | agttgcgtgattcatctggtaaaactattagtac |
| | atggatttcagatggacatgtgaaggatttctac |
| | ctgtatccaggaaaatatacatttgtcgaaaccg |
| | cagcaccagacggttatgaggtagcaactccaat |
| | tgaatttacagttaatgaggacggtcaggttact |
| | gtagatggtgaagcaactgaaggtgacgctcata |
| | ctggatccagtggtagc |

### Miscellaneous

In some embodiments, in addition to the transmembrane moiety or the immune checkpoint moiety, the composition can comprise a selection tag. In some embodiments, the composition described herein comprise one or more selection tags. In some embodiments, the selection tag is puromycin, hygromycin, neomycin, or blasticidin. In some embodiments, the selection tag is fluorescent protein. For example, the selection tag is green fluorescent protein, red fluorescent protein, blue fluorescent protein, or yellow fluorescent protein. In some embodiments, the selection tag is green fluorescent protein, such as mNeoGreen, mEGFP, Clover, Emerald, mEmerald, GFPmut2, GFPmut3, moxGFP, usGFP, muGFP, Sapphire, T-Sapphire, Topaz, SGFP1, SGFP2, cfSGFP2. In preferred embodiments, the selection marker is mNeonGreen. In some cases, the selection tag is PuroR or mNeonGreen. In some embodiments, the selection tag comprises a polypeptide sequence that is at least 50%, 55%, 60%, 65%, 70%, 75%, 80%, 85%, 90%, 95% or 99% identical to any one of SEQ ID NOS: 16-17. In some embodiments, the selection tag comprises a polypeptide sequence that is at least 50%, 55%, 60%, 65%, 70%, 75%, 80%, 85%, 90%, 95% or 99% identical to any one of SEQ ID NOS: 18-19.

TABLE 5
Exemplary selection tag sequences

| | |
|---|---|
| SEQ ID. 16 | PuroR |
| | atgaccgagtacaagcccacggtgcgcctcgccacccgcgacgacgtccccagggccgtacgcaccctcgc |
| | cgccgcgttcgccgactaccccgccacgcgccacaccgtcgatccggaccgccacatcgaggggtcaccg |
| | agctgcaagaactcttcctcacgcgcgtcgggctcgacatcggcaaggtgtgggtcgcggacgacggcgccg |
| | cggtggcggtctggaccacgccggagagcgtcgaagcgggggcggtgttcgccgagatcggcccgcgcat |
| | ggccgagttgagcggttcccggctggccgcgcagcaacagatggaaggcctcctggcgccgcaccggccca |
| | aggagcccgcgtggttcctggccaccgtcggcgtctcgcccgaccaccagggcaagggtctgggcagcgcc |
| | gtcgtgctccccggagtggaggcggccgagcgcgccggggtgcccgccttcctggagacctccgcgccccg |
| | caacctccccttctacgagcggctcggcttcaccgtcaccgccgacgtcgaggtgcccgaaggaccgcgcac |
| | ctggtgcatgacccgcaagcccggtgcc |
| SEQ ID. 17 | MTEYKPTVRLATRDDVPRAVRTLAAAFADYPATRHTVDPDRHIER VT |
| | ELQELFLTRVGLDIGKVWVADDGAAVAVWTTPESVEAGAVFAEIGPR |
| | MAELSGSRLAAQQQMEGLLAPHRPKEPAWFLATVGVSPDHQGKGLG |
| | SAVVLPGVEAAERAGVPAFLETSAPRNLPFYERLGFTVTADVEVPEGP |
| | RTWCMTRKPGA |
| | mNeonGreen |
| SEQ ID. 18 | atggtgagcaagggcgaggaggataacatggcctctctcccagcgacacatgagttacacatctttggctccat |
| | caacggtgtggactttgacatggtgggtcagggcaccggcaatccaaatgatggttatgaggagttaaacctga |
| | agtccaccaagggtgacctccagttctccccctggattctggtccctcatatcgggtatggcttccatcagtacctg |
| | ccctaccctgacgggatgtcgcctttccaggccgccatggtagatggctccggctaccaagtccatcgcacaat |
| | gcagtttgaagatggtgcctcccttactgttaactaccgctacacctacgagggaagccacatcaaaggagagg |
| | cccaggtgaaggggactggtttccctgctgacggtcctgtgatgaccaactcgctgaccgctgcggactggtgc |
| | aggtcgaagaagacttaccccaacgacaaaaccatcatcagtacctttaagtggagttacaccactggaaatgg |
| | caagcgctaccggagcactgcgcggaccacctacacctttgccaagccaatggcggctaactatctgaagaac |
| | cagccgatgtacgtgttccgtaagacggagctcaagcactccaagaccgagctcaacttcaaggagtggcaaa |
| | aggcctttaccgatgtgatgggcatggacgagctgtacaag |
| SEQ ID. 19 | MVSKGEEDNMASLPATHELHIFGSINGVDFDMVGQGTGNPNDGYEEL |
| | NLKSTKGDLQFSPWILVPHIGYGFHQYLPYPDGMSPFQAAMVDGSGY |
| | QVHRTMQFEDGASLTVNYRYTYEGSHIKGEAQVKGTGFPADGPVMT |
| | NSLTAADWCRSKKTYPNDKTIISTFKWSYTTGNGKRYRSTARTTYTFA |
| | KPMAANYLKNQPMYVFRKTELKHSKTELNFKEWQKAFTDVMGMDELYK |

In some embodiments, in addition to the transmembrane moiety, the immune checkpoint moiety, and/or the selection tag, the composition can further comprise a self-cleavage peptide. In some embodiments, the composition described herein comprise one or more self-cleavage peptides. In some cases, the self-cleavage peptide is GSG-T2A. In some embodiments, the self-cleavage peptide comprises a polypeptide sequence that is at least 50%, 55%, 60%, 65%, 70%, 75%, 80%, 85%, 90%, 95% or 99% identical to SEQ ID NO: 22. In some embodiments, the self-cleavage peptide comprises a polypeptide sequence that is at least 50%, 55%, 60%, 65%, 70%, 75%, 80%, 85%, 90%, 95% or 99% identical to SEQ ID NO: 23.

TABLE 6
Exemplary self-cleavage peptide sequences

| | GSG T2A |
|---|---|
| SEQ ID. | ggcagcggagagggcagaggaagtcttctaacatgcggtg |
| 22 | acgtggaggagaatcccggccctaggctcgag |
| SEQ ID. | GSGEGRGSLLTCGDVEENPGPRLE |
| 23 | |

In some embodiments, in addition to the transmembrane moiety, the immune checkpoint moiety, the selection tag, and/or the self-cleavage peptide, the composition can comprise a glycosylation motif. In some embodiments, the composition described herein comprise one or more glycosylation motifs. In some embodiments, the glycosylation motif comprises a polypeptide sequence that is at least 50%, 55%, 60%, 65%, 70%, 75%, 80%, 85%, 90%, 95%, or 99% identical to SEQ ID NO: 24. In some embodiments, the glycosylation motif comprises a polypeptide sequence that is at least 50%, 55%, 60%, 65%, 70%, 75%, 80%, 85%, 90%, 95%, or 99% identical to SEQ ID NO: 25.

TABLE 7
Exemplary glycosylation motif sequences

| | |
|---|---|
| | GNSTM Glycosylation motif |
| SEQ ID. 24 | ggtaactcgactatg |
| SEQ ID. 25 | GNSTM |

In some embodiments, in addition to the transmembrane moiety, the immune checkpoint moiety, the selection tag, the self-cleavage peptide, and/or the glycosylation motif, the composition can comprise a spacer. In some embodiments, the composition described herein comprise one or more spacers. In some embodiments, the spacer comprises a polypeptide sequence that is at least 50%, 55%, 60%, 65%, 70%, 75%, 80%, 85%, 90%, 95%, or 99% identical to SEQ ID NO: 26. In some embodiments, the spacer comprises a polypeptide sequence that is at least 50%, 55%, 60%, 65%, 70%, 75%, 80%, 85%, 90%, 95%, or 99% identical to SEQ ID NO: 27.

TABLE 8
Exemplary spacer sequences

| | |
|---|---|
| SEQ ID. 26 | ggcagtggatctggatccggtggctcgagt |
| SEQ ID. 27 | GSGSGSGGSS |

In some embodiments, in addition to the transmembrane moiety, the immune checkpoint moiety, the selection tag, the self-cleavage peptide, the glycosylation motif, and/or the spacer, the composition can comprise gRNA. In some embodiments, the composition described herein comprise one or more gRNAs. In some cases, the gRNA is LAMP2B gRNA, PTGFR gNRA, or IGSF gNRA. In some embodiments, the gRNA comprises a polypeptide sequence that is at least 50%, 55%, 60%, 65%, 70%, 75%, 80%, 85%, 90%, 95%, or 99% identical to SEQ ID NO: 28. In some embodiments, the gRNA comprises a polypeptide sequence that is at least 50%, 55%, 60%, 65%, 70%, 75%, 80%, 85%, 90%, 95%, or 99% identical to SEQ ID NO: 29. In some embodiments, the gRNA comprises a polypeptide sequence that is at least 50%, 55%, 60%, 65%, 70%, 75%, 80%, 85%, 90%, 95%, or 99% identical to SEQ ID NO: 30.

TABLE 9
Exemplary gRNA sequences

| | |
|---|---|
| | LAMP2B gRNA |
| SEQ ID. 28 | AGCTGTGCGGTCTTATGCAT TGG |
| | PTGFR gRNA |
| SEQ ID. 29 | CAACCTGCCAGACGGAAAAC CGG |
| | IGSF8 gRNA |
| SEQ ID. 30 | CTCGGGGACCAGCACCTCCC GGG |

In some embodiments, in addition to the transmembrane moiety, the immune checkpoint moiety, the selection tag, the self-cleavage peptide, the glycosylation motif, the spacer and/or gRNA, the composition can comprise a linker. In some embodiments, the composition described herein comprise one or more linkers. In some embodiments, the linker is a flexible linker and encoded from a polypeptide sequence that is at least 50%, 55%, 60%, 65%, 70%, 75%, 80%, 85%, 90%, 95%, or 99% identical to any one of SEQ ID NOS: 58-66. In some embodiments, the linker is a rigid linker and encoded from a polypeptide sequence that is at least 50%, 55%, 60%, 65%, 70%, 75%, 80%, 85%, 90%, 95%, or 99% identical to any one of SEQ ID NOS: 67-70. In some embodiments, the linker is a cleavable linker and encoded from a polypeptide sequence that is at least 50%, 55%, 60%, 65%, 70%, 75%, 80%, 85%, 90%, 95%, or 99% identical to any one of SEQ ID NOS: 71-74.

In some embodiments, the transmembrane moiety is complexed with the immune checkpoint moiety described herein to generate the fusion proteins or polypeptides described herein. In some embodiments, the transmembrane moiety is non-covalently complex with the immune checkpoint moiety described herein. In some embodiments, the transmembrane moiety is covalently complexed with the immune checkpoint moiety described herein. In some embodiments, the transmembrane moiety is fused to the immune checkpoint moiety described herein at the N-terminus of the transmembrane moiety. In some embodiments, the transmembrane moiety is fused to the immune checkpoint moiety described herein at the C-terminus of the transmembrane moiety. In some embodiments, the immune checkpoint moiety is inserted (e.g. via homology recombination) at any locus of the transmembrane moiety. In some embodiments, the transmembrane moiety is covalently connected to the immune checkpoint moiety by a peptidyl linker. In some cases, the linker is a flexible linker, a rigid linker, or a cleavable linker. In some embodiments, the flexible linker is (GGGGS)n where n=1, 2, 3, or 4; KESGSVSSEQLAQFRSLD (SEQ ID NO: 59); EGKSSGSGSESKST (SEQ ID NO: 60); GGGGGG (SEQ ID NO: 61); GGGGGGGG (SEQ ID NO: 62); GSAGSAAGSGEF (SEQ ID NO: 63); RKRR (SEQ ID NO: 64); SS (SEQ ID NO: 65); or LE (SEQ ID NO: 66). In some instances, the rigid linker is (EAAAK)n where n=1, 2, 3, or 4; A(EAAAK)nA where n=2, 3, 4, or 5; A(EAAAK)4ALEA(EAAAK)4A (SEQ ID NO: 68); AEAAAKEAAAKA (SEQ ID NO: 99); or LEAGCKNFFPRSFTSCGSLE (SEQ ID NO: 70). In some aspects, the cleavable linker is LEAGCKNFFPRSFTSCGSLE (SEQ ID NO: 71); CRRRRRREAEAC (SEQ ID NO: 72); GGIEGRGS (SEQ ID NO: 73); or TRHRQPRGWEQL (SEQ ID NO: 74).

TABLE 10
Exemplary linker sequences

| | |
|---|---|
| | Flexible linker |
| SEQ ID NO: 58 | GGGGS |
| SEQ ID NO: 59 | KESGSVSSEQLAQFRSLD |
| SEQ ID NO: 60 | EGKSSGSGSESKST |
| SEQ ID NO: 61 | GGGGGG |
| SEQ ID NO: 62 | GGGGGGGG |
| SEQ ID NO: 63 | GSAGSAAGSGEF |
| SEQ ID NO: 64 | RKRR |
| SEQ ID NO: 65 | SS |
| SEQ ID NO: 66 | LE |
| | Rigid linker |
| SEQ ID NO: 67 | EAAAK |
| SEQ ID NO: 68 | A(EAAAK)4ALEA(EAAAK)4A |
| SEQ ID NO: 69 | AEAAAKEAAAKA |
| SEQ ID NO: 70 | LEAGCKNFFPRSFTSCGSLE |
| | Cleavable linker |
| SEQ ID NO: 71 | LEAGCKNFFPRSFTSCGSLE |
| SEQ ID NO: 72 | CRRRRRREAEAC |
| SEQ ID NO: 73 | GGIEGRGS |
| SEQ ID NO: 74 | TRHRQPRGWEQL |

### Methods

### Generating Engineered Extracellular Vesicles

Described herein, in some embodiments, are methods for generating the compositions descried herein utilizing the platforms described herein. In some embodiments, described herein are methods for generating the engineered extracellular vesicles described herein. In some instances, the engineered extracellular vesicles is isolated from or released by a cell described herein. For example, engineered extracellular vesicles are generated from lysing the cells to release the engineered extracellular vesicles. In some cases, the cells secrete or release the engineered extracellular vesicles, where the engineered extracellular vesicles can then be isolated. An exemplary workflow for generating and isolating the engineered extracellular vesicles described herein is illustrated in FIG. 6.

In some embodiments, the cells for generating the engineered extracellular vesicle are cells from cell lines, stem cells, primary cells, or differentiated cells. In some embodiments, the cells are selected from the group consisting of human embryonic fibroblasts (HEF), dendritic cells, mesenchymal stem cells, bone marrow-derived dendritic cells, bone marrow derived stromal cells, adipose stromal cells, endothelial cells, enucleated cells, neural stem cells, immature dendritic cells, and immune cells. bone marrow stromal cells, marrow derived adult progenitor cells (MAPCs), endothelial progenitor cells (EPC), blast cells, intermediate progenitor cells formed in the subventricular zone, neural stem cells, muscle stem cells, satellite cells, liver stem cells, hematopoietic stem cells, bone marrow stromal cells, epidermal stem cells, embryonic stem cells, umbilical cord stem cells, precursor cells, muscle precursor cells, myoblast, cardiomyoblast, neural precursor cells, glial precursor cells, neuronal precursor cells, or hepatoblasts.

In some embodiments, the cell for generating the engineered extracellular vesicles is a genetically modified cell, where a genetic modification moiety is introduced into the modified cell. In some embodiments, at least one heterologous polynucleotide encoding any one of the immune checkpoint moieties described herein is introduced into the modified cell. In some embodiments, the heterologous polynucleotide encodes any one of the targeting moieties described herein. In some embodiments, the heterologous polynucleotide encodes any one of the transmembrane moieties described herein. In some embodiments, the heterologous polynucleotide encodes any one of the fusion peptides described herein. In some embodiments, the heterologous polynucleotide encodes any one of the immune evasion moieties described herein. In some embodiments, the heterologous polynucleotide is integrated into the chromosome of the modified cell. In some embodiments, the heterologous polynucleotide is not integrated into the chromosome of the modified cell. In some embodiments, the heterologous polynucleotide is a vector or plasmid comprising nucleic acid sequence encoding the transmembrane moiety. In some embodiments, the heterologous polynucleotide is a vector or plasmid comprising nucleic acid sequences encoding both the transmembrane moiety and the immune checkpoint moiety. In some embodiments, the heterologous polynucleotide is a vector or plasmid comprising nucleic acid sequences encoding the immune checkpoint moiety being flanked by the transmembrane moiety (FIG. 5). In such case, the immune checkpoint moiety is inserted via homologous recombination.

In some embodiments, the genetic modification moiety regulates the expressions of the heterologous polynucleotide encoding the transmembrane moiety and the immune checkpoint moiety. In some embodiments, the genetic modification moiety increases the expressions of the heterologous polynucleotide. In some embodiments, the genetic modification moiety comprises a CRISPR-Cas polypeptide. In some embodiments, the genetic modification moiety is, for example, Class 1 CRISPR-associated (Cas) polypeptides, Class 2 Cas polypeptides, type I Cas polypeptides, type II Cas polypeptides, type III Cas polypeptides, type IV Cas polypeptides, type V Cas polypeptides, and type VI, CRISPR-associated RNA binding proteins, or a functional fragment thereof. Cas polypeptides suitable for use with the present disclosure can include Cas9, Cas12, Cas13, Cpf1 (or Cas12a), C2C1, C2C2 (or Cas13a), Cas13b, Cas13c, Cas13d, C2C3, Cas1, Cas1B, Cas2, Cas3, Cas4, Cas5, Cas5e (CasD), Cas6, Cas6e, Cas6f, Cas7, Cas8a, Cas8a1, Cas8a2, Cas8b, Cas8c, Csn1, Csx12, Cas10, Cas10d, Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cul966; any derivative thereof, any variant thereof; or any fragment thereof. In some embodiments, Cas13 can include, but are not limited to, Cas13a, Cas13b, Cas13c, and Cas 13d (e.g., CasRx). CRISPR/Cas can be DNA and/or RNA cleaving or can exhibit reduced cleavage activity. Genetic modification moiety can be configured to complex with at least one heterologous RNA polynucleotide. In some cases, the genetic modification moiety can be fused with a transcription activator or transcription repressor.

Any suitable nuclease (e.g., endonuclease) can be used in as the genetic modification moiety. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute (Ago) proteins (e.g., prokaryotic Argonaute (pAgo), archaeal Argonaute (aAgo), eukaryotic Argonaute (eAgo), and Natronobacterium gregoryi Argonaute (NgAgo)); Adenosine deaminases acting on RNA (ADAR); CIRT, PUF, homing endonuclease, or any functional fragment thereof, any derivative thereof, any variant thereof, and any fragment thereof.

In some embodiments, the genetic modification moiety is CRISPR/Cas9. In some embodiments, the CRISPR/Cas9 cleaves the nucleic acid sequence encoding the transmembrane moiety. Such cleavage event allows the heterologous polynucleotide encoding the immune checkpoint moiety to be inserted into a locus of the transmembrane moiety via homologous recombination.

A genetic modification moiety as disclosed herein can be coupled (e.g., linked or fused) to additional peptide sequences which are not involved in regulating gene expression, for example linker sequences, targeting sequences, etc. The term “targeting sequence,” as used herein, refers to a nucleotide sequence and the corresponding amino acid sequence which encodes a targeting polypeptide which mediates the localization (or retention) of a protein to a sub-cellular location, e.g., plasma membrane or membrane of a given organelle, nucleus, cytosol, mitochondria, endoplasmic reticulum (ER), Golgi, chloroplast, apoplast, peroxisome or other organelle. For example, a targeting sequence can direct a protein (e.g., a receptor polypeptide or an adaptor polypeptide) to a nucleus utilizing a nuclear localization signal (NLS); outside of a nucleus of a cell, for example to the cytoplasm, utilizing a nuclear export signal (NES); mitochondria utilizing a mitochondrial targeting signal; the endoplasmic reticulum (ER) utilizing an ER-retention signal; a peroxisome utilizing a peroxisomal targeting signal; plasma membrane utilizing a membrane localization signal; or combinations thereof.

A genetic modification moiety as disclosed herein can be a part of a fusion construct (e.g., a fusion protein). As used herein, “fusion” can refer to a protein and/or nucleic acid comprising one or more non-native sequences (e.g., moieties). A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-directed polypeptide (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as Alexa fluor dyes, Cyanine3 dye, Cyanine5 dye.

A fusion can refer to any protein with a functional effect. For example, a fusion protein can comprise methyltransferase activity, demethylase activity, dismutase activity, alkylation activity, depurination activity, oxidation activity, pyrimidine dimer forming activity, integrase activity, transposase activity, recombinase activity, polymerase activity (e.g., a reverse transcriptase activity), ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodelling activity, protease activity, oxidoreductase activity, transferase activity, hydrolase activity, lyase activity, isomerase activity, synthase activity, synthetase activity, or demyristoylation activity. An effector protein can modify a genomic locus. A fusion protein can be a fusion in a Cas protein. A fusion protein can be a non-native sequence in a Cas protein.

In some embodiments, the genetic modification moiety is fused to one or more transcription repressor domains, activator domains, epigenetic domains, recombinase domains, transposase domains, flippase domains, nickase domains, or any combination thereof. The activator domain can include one or more tandem activation domains located at the carboxyl terminus of the protein. In some cases, the genetic modification moiety includes one or more tandem repressor domains located at the carboxyl terminus of the protein. Non-limiting exemplary activation domains include GAL4, herpes simplex activation domain VP16, VP64 (a tetramer of the herpes simplex activation domain VP16), NF-κB p65 subunit, Epstein-Barr virus R transactivator (Rta). Non-limiting exemplary repression domains include the KRAB (Kruppel-associated box) domain of Kox1, the Mad mSIN3 interaction domain (SID), or ERF repressor domain (ERD). In some embodiments, the genetic modification moiety includes one or more tandem repressor domains located at the amino terminus of the protein.

In some embodiments, the nuclease disclosed herein is a protein that lacks nucleic acid cleavage activity. In some cases, a Cas protein is a dead Cas protein. A dead Cas protein comprises a protein that lacks nucleic acid cleavage activity. A Cas protein can comprise a modified form of a wild type Cas protein. The modified form of the wild type Cas protein can comprise an amino acid change (e.g., deletion, insertion, or substitution) that reduces the nucleic acid-cleaving activity of the Cas protein. For example, the modified form of the Cas protein can have less than 90%, less than 80%, less than 70%, less than 60%, less than 50%, less than 40%, less than 30%, less than 20%, less than 10%, less than 5%, or less than 1% of the nucleic acid-cleaving activity of the wild-type Cas protein (e.g., Cas9 from S. pyogenes). The modified form of Cas protein can have no substantial nucleic acid-cleaving activity. When a Cas protein is a modified form that has no substantial nucleic acid-cleaving activity, it can be referred to as enzymatically inactive and/or “dead” (abbreviated by “d”). A dead Cas protein (e.g., dCas, dCas9) can bind to a target polynucleotide but may not cleave the target polynucleotide. In some aspects, a dead Cas protein is a dead Cas9 protein.

In some embodiments, a dCas (e.g., dCas9) polypeptide can associate with a single guide RNA (sgRNA) to activate or repress transcription of target DNA. sgRNAs can be introduced into cells expressing the engineered chimeric receptor polypeptide. In some cases, such cells contain one or more different sgRNAs that target the same nucleic acid. In other cases, the sgRNAs target different nucleic acids in the cell.

In some embodiments, the genetic modification moiety can comprise a catalytically inactive Cas polypeptide, where the nuclease activity of the Cas polypeptide is eliminated or substantially eliminated.

In some instances, the genetic modification moiety can comprise a catalytically inactivated Cas9 (dCas9), any derivative thereof; any variant thereof; or any fragment thereof.

In some instances, the genetic modification moiety can comprise a catalytically inactivated Cas12 (dCas12), any derivative thereof, any variant thereof, or any fragment thereof.

In some instances, the genetic modification moiety can comprise a catalytically inactivated Cas13 (dCas13); any derivative thereof, any variant thereof; or any fragment thereof.

In some embodiments, the genetic modification moiety is complexed with the at least one heterologous polynucleotide as described herein. In some embodiments, the at least one heterologous polynucleotide is either heterologous DNA polynucleotide or heterologous RNA polynucleotide. In some embodiments, the genetic modification moiety is complexed with at least one heterologous RNA polynucleotide. In some embodiments, the complexing with the at least one heterologous RNA polynucleotide direct and target the genetic modification moiety to the portion of the heterologous polynucleotide.

In some cases, the compositions and methods described herein comprise at least one heterologous polynucleotide. In some cases, the compositions and methods described herein comprise a plurality of heterologous nucleic acids. In some embodiments, the polynucleotide is deoxyribonucleic acid (DNA). In some cases, the DNA sequence is single-stranded or doubled-stranded. In some embodiments, the at least one heterologous nucleic acid polynucleotide is ribonucleic acid (RNA).

In some embodiments, the genetic modification moiety is complexed with the at least one heterologous RNA polynucleotide. The at least one heterologous RNA polynucleotide can comprise a nucleic-acid targeting region that comprises a complementary sequence to a nucleic acid sequence of the heterologous polynucleotide that encodes any one of the moieties described herein for specificity of the genetic modification moiety-dependent targeting. In some embodiments, the at least one heterologous RNA polynucleotide is guide nucleic acid (or guide RNA) comprising two separate nucleic acid molecules, which is referred to as a double guide nucleic acid or a single nucleic acid molecule, which is referred to as a single guide nucleic acid (e.g., sgRNA). In some embodiments, the guide nucleic acid is a single guide nucleic acid comprising a fused CRISPR RNA (crRNA) and a transactivating crRNA (tracrRNA). In some embodiments, the guide nucleic acid is a single guide nucleic acid comprising a crRNA. In some embodiments, the guide nucleic acid is a single guide nucleic acid comprising a crRNA but lacking a tracrRNA. In some embodiments, the guide nucleic acid is a double guide nucleic acid comprising non-fused crRNA and tracrRNA. An exemplary double guide nucleic acid can comprise a crRNA-like molecule and a tracrRNA-like molecule. An exemplary single guide nucleic acid can comprise a crRNA-like molecule. An exemplary single guide nucleic acid can comprise a fused crRNA-like molecule and a tracrRNA-like molecule.

A crRNA can comprise the nucleic acid-targeting segment (e.g., spacer region) of the guide nucleic acid and a stretch of nucleotides that can form one half of a double-stranded duplex of the Cas protein-binding segment of the guide nucleic acid.

A tracrRNA can comprise a stretch of nucleotides that forms the other half of the double-stranded duplex of the Cas protein-binding segment of the gRNA. A stretch of nucleotides of a crRNA can be complementary to and hybridize with a stretch of nucleotides of a tracrRNA to form the double-stranded duplex of the Cas protein-binding domain of the guide nucleic acid.

The crRNA and tracrRNA can hybridize to form a guide nucleic acid. The crRNA can also provide a single-stranded nucleic acid targeting segment (e.g., a spacer region) that hybridizes to a target nucleic acid recognition sequence (e.g., protospacer). The sequence of a crRNA, including spacer region, or tracrRNA molecule can be designed to be specific to the species in which the guide nucleic acid is to be used. In some embodiments, the nucleic acid-targeting region of a guide nucleic acid can be between 18 to 72 nucleotides in length.

The nucleotide sequence of the guide nucleic acid that is complementary to a nucleotide sequence (target sequence) of the target nucleic acid can have a length of, for example, at least about 12 nt, at least about 15 nt, at least about 18 nt, at least about 19 nt, at least about 20 nt, at least about 25 nt, at least about 30 nt, at least about 35 nt or at least about 40 nt. The nucleotide sequence of the guide nucleic acid that is complementary to a nucleotide sequence (target sequence) of the target nucleic acid can have a length of from about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the guide nucleic acid (e.g. guide RNA or gRNA) can direct the CRISPR/Cas9 described herein to cleave the transmembrane moiety to induce homologous recombination in order to insert the immune checkpoint moiety in the flanking regions of the transmembrane moiety.

A protospacer sequence of a targeted polynucleotide can be identified by identifying a PAM within a region of interest and selecting a region of a desired size upstream or downstream of the PAM as the protospacer. A corresponding spacer sequence can be designed by determining the complementary sequence of the protospacer region.

A spacer sequence can be identified using a computer program (e.g., machine readable code). The computer program can use variables such as predicted melting temperature, secondary structure formation, and predicted annealing temperature, sequence identity, genomic context, chromatin accessibility, % GC, frequency of genomic occurrence, methylation status, presence of SNPs, and the like.

The percent complementarity between the nucleic acid-targeting sequence (e.g., a spacer sequence of the at least one heterologous polypeptide as disclosed herein) and the target nucleic acid (e.g., a protospacer sequence of the heterologous polynucleotide encoding any one of the moieties described herein) can be at least 50%, at least 60%, at least 70%, at least 75%, at least 80%, at least 85%, at least 90%, at least 95%, at least 97%, at least 98%, at least 99%, or 100%. The percent complementarity between the nucleic acid-targeting sequence and the target nucleic acid can be at least 60%, at least 70%, at least 75%, at least 80%, at least 85%, at least 90%, at least 95%, at least 97%, at least 98%, at least 99%, or 100% over about 20 contiguous nucleotides.

The Cas protein-binding segment of a guide nucleic acid can comprise two stretches of nucleotides (e.g., crRNA and tracrRNA) that are complementary to one another. The two stretches of nucleotides (e.g., crRNA and tracrRNA) that are complementary to one another can be covalently linked by intervening nucleotides (e.g., a linker in the case of a single guide nucleic acid). The two stretches of nucleotides (e.g., crRNA and tracrRNA) that are complementary to one another can hybridize to form a double stranded RNA duplex or hairpin of the Cas protein-binding segment, thus resulting in a stem-loop structure. The crRNA and the tracrRNA can be covalently linked via the 3′ end of the crRNA and the 5′ end of the tracrRNA. Alternatively, tracrRNA and crRNA can be covalently linked via the 5′ end of the tracrRNA and the 3′ end of the crRNA.

In some embodiments, the genetic modification moiety and the heterologous polynucleotide are delivered into the cell via the use of expression vectors. In the context of an expression vector, the vector can be readily introduced into a host cell, e.g., mammalian, bacterial, yeast, or insect cell by any method in the art. For example, the expression vector can be transferred into a host cell by physical, chemical, or biological means. In some embodiments, the genetic modification moiety and the heterologous polynucleotide are delivered into the cell via physical methods such as calcium phosphate precipitation, lipofection, particle bombardment, microinjection, gene gun, electroporation, and the like. Methods for producing cells comprising vectors and/or exogenous nucleic acids are suitable for methods herein. One method for the introduction of a polynucleotide into a host cell is calcium phosphate transfection. In some embodiments, the genetic modification moiety and the heterologous polynucleotide are delivered into the cell via biological methods such as the use of DNA and RNA vectors. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAVs), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV vectors include AAV1, AAV2, AAV4, AAV5, AAV6, AAV7, AAV8, or AAV9 serotype. In some instances, viral vector is a chimeric viral vector, comprising viral portions from two or more viruses. In additional instances, the viral vector is a recombinant viral vector. In some embodiments, the genetic modification moiety and the heterologous polynucleotide are delivered into the cell via chemical means such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system. In some embodiments, the genetic modification moiety and the heterologous polynucleotide are delivered into the cell via a non-viral delivery system. Non-viral delivery system can be liposome. The use of lipid formulations is contemplated for the introduction of the nucleic acids into a host cell (in vitro, ex vivo or in vivo). In another aspect, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes. In some embodiments, the genetic modification moiety and the heterologous polynucleotide are delivered into the cell are packaged and delivered to the cell via extracellular vesicles. The engineered extracellular vesicles can be any membrane-bound particles. In some embodiments, the engineered extracellular vesicles are any membrane-bound particles secreted by at least one cell. In some instances, the engineered extracellular vesicles are any membrane-bound particles synthesized in vitro. In some instances, the engineered extracellular vesicles are any membrane-bound particles synthesized without a cell. In some cases, the engineered extracellular vesicles are exosomes, microvesicles, retrovirus-like particles, apoptotic bodies, apoptosomes, oncosomes, exophers, enveloped viruses, exomeres, or other very large extracellular vesicles.

### Identifying and Isolating Homogeneous or Heterogenous Populations of Engineered Extracellular Vesicles

Described herein, in some embodiments, are methods for utilizing the platforms described herein to generate compositions comprising a homogeneous or a heterogenous population of engineered extracellular vesicles. In some embodiments, the method identifies and isolates the homogeneous or the heterogenous population of engineered extracellular vesicles based on the dimensions (e.g. diameters or sizes) of the engineered extracellular vesicles. In some embodiments, the method identifies and isolates the homogeneous or the heterogenous population of engineered extracellular vesicles based on the mass of the engineered extracellular vesicles. In some embodiments, the method identifies and isolates the homogeneous or the heterogenous population of engineered extracellular vesicles based on the number of units of immune checkpoint moiety encapsulated, secreted, released, or expressed on the surface of the engineered extracellular vesicle. In some embodiments, the method identifies and isolates the homogeneous or the heterogenous population of engineered extracellular vesicles based on a combination of the dimensions and the number of units if immune checkpoint moiety encapsulated, secreted, or expressed on the surface of the extrasellar vesicle. In some embodiments, the method identifies and isolates the homogeneous or the heterogenous population of engineered extracellular vesicles based on a combination of dimensions and the number of units of immune checkpoint moiety expressed on the surface of the engineered extracellular vesicle.

In some embodiments, the method of identifying and isolating the homogeneous or the heterogenous population of engineered extracellular vesicles comprises performing differential ultracentrifugation to isolate a homogeneous or a heterogenous population of engineered extracellular vesicle based on density. In some embodiments the method comprises performing filtration or ultrafiltration to isolate homogeneous or heterogenous population of engineered extracellular vesicles based on weights or sizes. In some embodiments, the method comprises performing HPLC. In some embodiments, the method comprises performing extracellular vesicle precipitation, where water-excluding polymers such as polyethylene glycol (PEG) can tie up water molecules and force less soluble components out of solution. As such, the precipitate containing extracellular vesicle cam ne isolated by means of either low-speed centrifugation or filtration. In some embodiments, the method comprises performing affinity-based capture by capturing the engineered extracellular vesicles by immunoaffinity. Examples of proteins or epitope displayed on the surface of the engineered extracellular vesicles include CD9, CD63. CD81. Alix, caveolin-1, CD41, CD4, flotillin, Rab5, HSC70, and Lamp-3. In some embodiments, the method comprises performing microfluidics-based isolation method for extracellular vesicle for identifying and isolating a homogeneous or a heterogenous population of engineered extracellular vesicle based on size, density, and immunoaffinity, innovative sorting mechanisms such as acoustic, electrophoretic and electromagnetic manipulations are implemented. With the use of such devices, significant reductions in sample volume, reagent consumption, and isolation time are expected.

In some embodiments, the method of identifying and isolating the homogeneous or the heterogenous population of engineered extracellular vesicles comprises basing on the number of immune checkpoint moiety expressed on the surface of the engineered extracellular vesicle. In some embodiments, the method comprises immunoassay, where antibody recognizing the immune checkpoint moiety is used. In some embodiments, the antibody is conjugated to a detectable moiety. In some embodiments, the signal detected from the antibody recognizing and binding to the immune checkpoint moiety correlates with the number of immune checkpoint moiety expressed on the surface of the engineered extracellular vesicle. Exemplary detectable moiety includes an enzymatic moiety (e.g., horseradish peroxidase (HRP), beta-galactosidase, alkaline phosphatase, etc), fluorescent dye, luminescent moiety, radioactive moiety, colorimetric label, colored latex particle or nanoparticle, and metal-conjugated moiety such as metallic nanolayer, metallic nanoparticle, or metallic nanoshell-conjugated moiety. In some embodiments, the detectable moiety is directly or indirectly tagged for a colorimetric assay (e.g., for detection of HRP or beta-galactosidase activity), visual inspection using light microscopy, immunofluorescence microscopy, confocal microscopy, by flow cytometry (FACS), autoradiography electron microscopy, immunostaining, or subcellular fractionation.

In some embodiments, the method of identifying and isolating the homogeneous or a heterogenous population of engineered extracellular vehicles comprises identifying and isolating the homogeneous or a heterogenous population of engineered extracellular vesicles based on both diameter and number of units of immune checkpoint moiety expressed on the surface of the engineered extracellular vesicle. For example, the method identifies and isolates a homogeneous or a heterogenous population of engineered extracellular vesicles comprising a diameter of about 50 nm and about 2000 units of immune checkpoint moiety expressed on the surface of the engineered extracellular vesicles. In some embodiments, the method identifies and isolates a homogeneous or a heterogenous population of engineered extracellular vesicles comprising a diameter of about 10 nm, 20 nm, 30 nm, 40 nm, 50 nm, 60 nm, 70, nm, 80 nm, 90 nm, 100 nm, 110 nm, 120 nm, 130 nm, 140 nm, 150 nm, 160 nm, 170 nm, 180 nm, 190 nm, 200 nm, or more and 500 units, 1000 units, 1500 units, 2000 units, 2500 units, 3000 units, 3500 units, 4000 units, 4500 units, 5000 units, 5500 units, 6000 units, 7500 units, 8000 units, 8500 units, 9000 units, 9500 units, 10000 units, 11000 nuts, 12000 units, 13000 units, 14000 units, 15000 units, or more of the immune checkpoint expressed on the surface of the engineered extracellular vesicle.

In some embodiments, the method of identifying and isolating the homogenous or a heterogenous population of engineered extracellular vehicles comprises identifying and isolating the homogenous or a heterogenous population of engineered extracellular vesicles based on a selection marker fused to the engineered extracellular vehicles. Examples of the selection marker include, without limitation, puromycin, hygromycin, neomycin, and blasticidin. In preferred embodiments, the selection marker is puromycin. For example, the puromycin fused to the engineered extracellular vehicles can be detected by standard immunoblotting assay, enzyme-linked immunosorbent assay (ELISA), or heterologous enzyme immunoassay (EIA). In some embodiments, the selection marker described herein is fluorescent protein. Examples of the florescent protein include, without limitation, green fluorescent protein, red fluorescent protein, blue fluorescent protein, and yellow fluorescent protein. In some embodiments, the selection marker is green fluorescent protein, such as mNeoGreen, mEGFP, Clover, Emerald, mEmerald, GFPmut2, GFPmut3, moxGFP, usGFP, muGFP, Sapphire, T-Sapphire, Topaz, SGFP1, SGFP2, cfSGFP2. In preferred embodiments, the selection marker is mNeonGreen. For example, fluorescent protein fused to the engineered extracellular vehicles can be detected by microscopy, western blot analysis, enzyme-linked immunosorbent assay (ELISA),

### Treatment

Disclosed herein, in some embodiments, are methods of treating a disease or a disorder in a subject, comprising administrating of therapeutic effective amount of the compositions or pharmaceutical compositions described herein to the subject. In some embodiments, the disease or disorder is an autoimmune disease, including Rheumatoid arthritis, Systemic lupus erythematosus, Psoriasis, Type 1 diabetes mellitus, Multiple sclerosis, Inflammatory bowel disease, Celiac disease, Crohn's disease, Graves' disease, Juvenile arthritis, Lyme disease chronic, Optic neuritis, Psoriatic arthritis, Scleritis, Scleroderma, Ulcerative colitis (UC), Uveitis, Inflammatory eye conditions, Vitiligo, COPD, complication from Organ transplantation, or graft-versus-host disease. In some cases, the disease or disorder is Acute Respiratory Distress Syndrome (ARDS). In some cases, the ARDS is caused by infection of coronavirus. In some cases, the coronavirus is SARS-CoV-2 (FIG. 2).

Disclosed herein, in some embodiments, are methods of treating a disease or a disorder in a subject, comprising administrating a therapeutic effective amount of the compositions or pharmaceutical compositions described herein (e.g., comprising an engineered extracellular vesicle) to the subject.

In some embodiments, the disease or disorder comprises multiple sclerosis (MS). MS is a serious inflammatory and neurodegenerative disease of the brain, spinal cord, and optic nerves that may result in 1 or more episodes of neurologic dysfunction with a variable course of recovery as well as disease progression. Central nervous system (CNS) lesions may be disseminated in time and space. MS usually has a long preclinical period with silent lesions on magnetic resonance imaging (MRI) at the time of clinical onset and subtle deficits on clinical testing may be present years before symptom onset. Disease activity may be evidenced by new or enlarging or enhancing lesions on MRI, new clinical manifestations, or progression of preexisting symptoms. MS is thought to be an autoimmune disease likely due to a combination of genetic predisposition and environmental influence.

In some embodiment, the extracellular vesicle comprises Brain-derived neurotrophic factor (BDNF) and CD200. In some embodiment, the extracellular vesicle comprises an extracellular domain of BDNF and an extracellular domain CD200.

In some embodiments, MS comprises progressive MS. MS may be characterized as progressive MS if clinical progression has occurred in the assessment period (usually annual). In some embodiments, progressive MS comprises primary progressive MS. In primary progressive MS, patients may have a steady decline in neurologic function from disease onset, without acute attacks. In some embodiments, progressive MS comprises secondary progressive MS. In secondary progressive MS, patients may initially have relapsing-remitting MS and then develop a progressive course later in the disease, with or without continued relapses.

In some embodiments, the method comprises steps of: contacting the cell with the compositions or pharmaceutical compositions as described herein; upon said contacting, the immune checkpoint moiety is delivered to the target cell. In some embodiments, the immune checkpoint moiety modulates immune response or of the target cell (FIG. 3). In some cases, the target cell comprises immune cells such as monocyte, T cell, Regulatory T cell (Treg), B cell, dendritic cell, macrophage, NK cell, or NKT cell. In some cases, the immune cell comprises cell that expresses CD8, CD25, or both CD8 and CD25 (CD8+CD25+). In some cases, the immune cell comprises cell that expresses CD4. In some cases, the immune cell comprises cell that expresses CD4 and CD 25 (CD4+CD25+). In some cases, the immune cell comprises cell that expresses FOXP3. In some cases, the immune cell comprises cell that expresses CD4, CD25, and FOXP3 (CD4+CD25+FOXP3+). In some embodiments, the contacting occurs in vivo, ex vivo, or in vitro. In some embodiment, the composition or pharmaceutical composition can directly be administered to the subject.

In some embodiments, the composition or pharmaceutical composition to be administered to the subject can comprise different combinations of the engineered extracellular vesicles expressing a plurality of immune checkpoint moiety. FIG. 7 illustrates exemplary combinations of the immune checkpoint moieties comprising PD-L1, CTLA-4, VISTA, and IGSF11 (VSIG-3). The therapeutic effects of the different combinations of the engineered extracellular vesicles can be determined by the methods in the examples described herein and in FIGS. 8-16.

In some embodiments, the composition or pharmaceutical composition is administered to the subject alone (e.g., standalone treatment). In some embodiments, the composition is administered in combination with an additional agent. In some embodiments, the composition is a first-line treatment for the disease or condition. In some embodiments, the composition is a second-line, third-line, or fourth-line treatment, for the autoimmune disease.

In general, methods disclosed herein comprise administering a composition by oral administration. However, in some instances, methods comprise administering a composition by intraperitoneal injection. In some instances, methods comprise administering a composition in the form of an anal suppository. In some instances, methods comprise administering a composition by intravenous (“i.v.”) administration. It is conceivable that one can also administer compositions disclosed herein by other routes, such as subcutaneous injection, intramuscular injection, intradermal injection, transdermal injection percutaneous administration, intranasal administration, intralymphatic injection, rectal administration intragastric administration, or any other suitable parenteral administration. In some embodiments, routes for local delivery closer to site of injury or inflammation are preferred over systemic routes. Routes, dosage, time points, and duration of administrating therapeutics can be adjusted. In some embodiments, administration of therapeutics is prior to, or after, onset of either, or both, acute and chronic symptoms of the disease or condition.

An effective dose and dosage of the compositions to prevent or treat the autoimmune diseases herein is defined by an observed beneficial response related to the autoimmune disease or condition, or symptom of the autoimmune disease. In some instances, the beneficial response comprises reduction of symptoms of autoimmune disease. Additional beneficial response comprises preventing, alleviating, arresting, or curing the autoimmune disease. In instances where the composition is not therapeutically effective or is not providing a sufficient alleviation of the disease or condition, or symptom of the disease or condition, then the dosage amount and/or route of administration can be changed, or an additional agent can be administered to the subject, along with the composition. In some embodiments, as a patient is started on a regimen of a composition, the patient is also weaned off (e.g., stepwise decrease in dose) a second treatment regimen.

Suitable dose and dosage administrated to a subject is determined by factors including, but no limited to, the particular composition, disease condition and its severity, the identity (e.g., weight, sex, or age) of the subject in need of treatment, and can be determined according to the particular circumstances surrounding the case, including, e.g., the specific agent being administered, the route of administration, the condition being treated, and the subject being treated.

In some embodiments, the administration of the composition is hourly, once every 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours 22 hours, 23 hours, 1 day, 2 days, 3 days, 4 days, 5 days, 6 days, 7 days, 8 days, 9 days, 10 days, 11 days, 12 days, 13 days, 14 days, 15 days, 1 month, 2 months, 3 months, 4 months, 5 months, 6 months, 7 months, 8 months, 9 months, 10 months, 11 months, 1 year, 2 years, 3 years, 4 years, or 5 years, or 10 years. The effective dosage ranges can be adjusted based on subject's response to the treatment. Some routes of administration will require higher concentrations of effective amount of therapeutics than other routes.

In certain embodiments, where the patient's condition does not improve, upon the doctor's discretion the administration of composition is administered chronically, that is, for an extended period of time, including throughout the duration of the patient's life in order to ameliorate or otherwise control or limit the symptoms of the patient's disease or condition. In certain embodiments wherein a patient's status does improve, the dose of composition being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered is temporarily reduced or temporarily suspended for a certain length of time (i.e., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, administration of the pharmaceutical composition lowers the occurrence of infection. In some embodiments, the incidence (e.g., average incidence) of infection is lowered by about 5% to about 10%, about 5% to about 15%, about 5% to about 20%, about 5% to about 25%, about 5% to about 30%, about 5% to about 40%, about 5% to about 50%, about 5% to about 60%, about 5% to about 70%, about 5% to about 80%, about 5% to about 90%, about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 20% to about 25%, about 20% to about 30%, about 20% to about 40%, about 20% to about 50%, about 20% to about 60%, about 20% to about 70%, about 20% to about 80%, about 20% to about 90%, about 25% to about 30%, about 25% to about 40%, about 25% to about 50%, about 25% to about 60%, about 25% to about 70%, about 25% to about 80%, about 25% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%.

In some embodiments, administration of the pharmaceutical composition results in a lower incidence of a sign or symptom associated with MS. For example, spasticity and muscle spasms, impaired mobility, upper extremity tremor, central neuropathic pain, fatigue, urinary symptoms, constipation, fecal incontinence, pseudobulbar effect, memory impairment, depression and/or anxiety, and combinations thereof. In some embodiments, administration of the pharmaceutical composition results in a lower incidence of a sign or symptom associated with MS by about 5% to about 10%, about 5% to about 15%, about 5% to about 20%, about 5% to about 25%, about 5% to about 30%, about 5% to about 40%, about 5% to about 50%, about 5% to about 60%, about 5% to about 70%, about 5% to about 80%, about 5% to about 90%, about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 20% to about 25%, about 20% to about 30%, about 20% to about 40%, about 20% to about 50%, about 20% to about 60%, about 20% to about 70%, about 20% to about 80%, about 20% to about 90%, about 25% to about 30%, about 25% to about 40%, about 25% to about 50%, about 25% to about 60%, about 25% to about 70%, about 25% to about 80%, about 25% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%.

In some embodiments, administration of the pharmaceutical composition results in a lower decelerated progression of neurologic disability. In some embodiments, administration of the pharmaceutical composition results in less (e.g., less progression) of lesions (e.g., T2-hyperintension lesions) (e.g., in the periventricular, cortical, juxtacortical, or infratentorial brain regions). In some embodiments, administration of the pharmaceutical composition results in less (e.g., less progression) of CSF-specific oligoclonal bands.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the composition described herein lies within a range of circulating concentrations that include the ED50 with minimal toxicity. In certain embodiments, the daily dosage range and/or the unit dosage amount varies within this range depending upon the dosage form employed and the route of administration utilized.

A composition can be used alone or in combination with an additional agent. In some cases, an “additional agent” as used herein is administered alone. The composition and the additional agent can be administered together or sequentially. The combination therapies can be administered within the same day, or can be administered one or more days, weeks, months, or years apart. Examples of additional agent can include other immune modulators such as antibodies targeting cytokines or small molecules.

### Platforms

Described herein, in some embodiments, are platforms for generating the engineered extracellular vesicles described herein (e.g., engineered exosomes). In some embodiments, the platforms conform to good manufacturing practices (GMP) standard. In some embodiments, the compositions comprising the engineered extracellular vesicle are generated according to good manufacturing practices (GMP). In some embodiments, the compositions comprise a pathogen level that is substantially free of pathogens. In some embodiments, the compositions comprise a contaminant level that is substantially free of contaminants. In some embodiments, the compositions comprise low immunogenicity.

In some embodiments, the compositions described herein are generated and isolated via hypotonic treatment and centrifugation. In some embodiments, the engineered extracellular vesicles are isolated from mesenchymal stem cells (MSCs) expressing the engineered extracellular vesicles primarily by using hypotonic treatment such that the MSCs rupture and extracellular vesicles are released. In some instances, the MSCs are resuspended in hypotonic solution to induce cell swelling. In some embodiments, the platform comprises phase-contrast microscopy to monitor cell swelling. In some embodiments, the platform comprises cell culturing modules. In some embodiments, the platform comprises modules for culturing the cells described herein. In some embodiments, the platform comprises modules for collecting the engineered extracellular vesicles released by the cells described herein. In some embodiments, the platform comprises a homogenizer to rupture the swollen cells to release extracellular vesicles. In some embodiments, the platform comprises means for separating the ruptured cells in a gradient (e.g., a sucrose gradient) to separate out the engineered extracellular vesicles. In some embodiments, the platform comprises other components to generate extracellular vesicles other approaches of lysing the MSC such as mild sonication, freeze-thaw, French-press, or needle-passaging. In some embodiments, the platform comprises centrifuges to centrifuge and isolate the fraction comprising the engineered extracellular vesicles. In some embodiments, the platform comprises means for separating a fraction comprising the engineered extracellular vesicle by floatation in a discontinuous sucrose density gradient.

In some embodiments, the platform comprises modules for generating the engineered extracellular vesicles by extrusion. In some embodiments, the extrusion process separates and isolates the engineered extracellular vesicles based on the sizes or diameters of the engineered extracellular vesicles. Exemplary extrusion process comprises the use of membranes with various pore sizes. The membranes can separate the engineered extracellular vesicles based on the sizes or diameters of the engineered extracellular vesicles from a solution comprising the ruptured MSC. Extracellular vesicles can be further isolated and reduced in size by continued extrusion following extrusion with increasingly smaller membrane pore sizes, ranging from 150 nm to 10 nm. When the final extrusion is complete, extracellular vesicle can be are pelleted by centrifugation. In some embodiments, the platform comprises components for performing sonication, extrusion, high pressure/homogenization, microfluidization, or detergent dialysis. In some embodiments, the platform comprises components for determining unit numbers of immune checkpoint moiety per extracellular vesicle.

### Pharmaceutical Compositions

Described herein, in some embodiments, are pharmaceutical compositions comprising the compositions described herein. In some cases, the pharmaceutical composition comprises the engineered extracellular vesicle described herein. In some embodiments, the pharmaceutical composition comprises both the composition comprising the engineered extracellular vesicle and the cells that release the engineered extracellular vesicles. A pharmaceutical composition, as used herein, refers to a mixture of a therapeutic agent comprising the engineered extracellular vesicle, with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more therapeutic agent (e.g., one or more therapeutic agents and one or more additional agents) as discussed herein. In practicing the methods of treatment or use provided herein, therapeutically effective amounts of therapeutic agents described herein are administered in a pharmaceutical composition to a mammal having a disease, disorder, or condition to be treated, e.g., an autoimmune disease. In some embodiments, the mammal is a human. A therapeutically effective amount can vary widely depending on the severity of the disease, the age and relative health of the subject, the potency of the therapeutic agent used and other factors. The therapeutic agents can be used singly or in combination with one or more therapeutic agents as components of mixtures.

The pharmaceutical formulations described herein are administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a therapeutic agent are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a therapeutic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, a therapeutic agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch. Formulations that include a therapeutic agent are prepared as solutions in saline, employing benzyl alcohol or other suitable preservatives, fluorocarbons, and/or other solubilizing or dispersing agents known in the art. Preferably these compositions and formulations are prepared with suitable nontoxic pharmaceutically acceptable ingredients. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the therapeutic agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, dyestuffs or pigments are added to the tablets or coatings for identification or to characterize different combinations of active therapeutic agent doses.

In some embodiments, pharmaceutical formulations of a therapeutic agent are in the form of a capsules, including push fit capsules made of gelatin, as well as soft, sealed capsules made of gelatin and a plasticizer, such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened, and the contents sprinkled on food prior to eating.

All formulations for oral administration are in dosages suitable for such administration. In one aspect, solid oral dosage forms are prepared by mixing a therapeutic agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of a therapeutic agent from the formulation. In other embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

Buccal formulations that include a therapeutic agent are administered using a variety of formulations known in the art. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formulated in a conventional manner.

For intravenous injections, a therapeutic agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

In some embodiments, the pharmaceutical dosage forms are formulated to provide a controlled release of a therapeutic agent. Controlled release refers to the release of the therapeutic agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein are delivered using a pulsatile dosage form. A pulsatile dosage form is capable of providing one or more immediate release pulses at predetermined time points after a controlled lag time or at specific sites. In one embodiment, the pulsatile dosage form includes at least two groups of particles, (i.e., multiparticulate) each containing the formulation described herein. The first group of particles provides a substantially immediate dose of a therapeutic agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a therapeutic agent and at least one dispersing agent or suspending agent for oral administration to a subject. The formulations may be powder and/or granule for suspension, and upon admixture with water, a substantially uniform suspension is obtained.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. Antimicrobial agents or preservatives can also be included in the formulation.

An aerosol formulation for inhalations and inhalants can be designed so that the agent or combination of agents is carried into the respiratory tree of the subject when administered by the nasal or oral respiratory route. Inhalation solutions can be administered, for example, by a nebulizer. Inhalations or insufflations, comprising finely powdered or liquid drugs, can be delivered to the respiratory system as a pharmaceutical aerosol of a solution or suspension of the agent or combination of agents in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants can include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Hydrocarbon propellants useful include, for example, propane, isobutane, n-butane, pentane, isopentane and neopentane. A blend of hydrocarbons can also be used as a propellant. Ether propellants include, for example, dimethyl ether as well as the ethers. An aerosol formulation can also comprise more than one propellant. For example, the aerosol formulation can comprise more than one propellant from the same class, such as two or more fluorocarbons; or more than one, more than two, more than three propellants from different classes, such as a fluorohydrocarbon and a hydrocarbon. Pharmaceutical compositions of the present disclosure can also be dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

The aerosol formulation can be packaged under pressure and can be formulated as an aerosol using solutions, suspensions, emulsions, powders and semisolid preparations. For example, a solution aerosol formulation can comprise a solution of an agent such as a transporter, carrier, or ion channel inhibitor in (substantially) pure propellant or as a mixture of propellant and solvent. The solvent can be used to dissolve the agent and/or retard the evaporation of the propellant. Solvents can include, for example, water, ethanol and glycols. Any combination of suitable solvents can be use, optionally combined with preservatives, antioxidants, and/or other aerosol components.

An aerosol formulation can be a dispersion or suspension. A suspension aerosol formulation can comprise a suspension of an agent or combination of agents, e.g., a transporter, carrier, or ion channel inhibitor, and a dispersing agent. Dispersing agents can include, for example, sorbitan trioleate, oleyl alcohol, oleic acid, lecithin and corn oil. A suspension aerosol formulation can also include lubricants, preservatives, antioxidant, and/or other aerosol components.

An aerosol formulation can similarly be formulated as an emulsion. An emulsion aerosol formulation can include, for example, an alcohol such as ethanol, a surfactant, water and a propellant, as well as an agent or combination of agents, e.g., a transporter, carrier, or ion channel. The surfactant used can be nonionic, anionic or cationic. One example of an emulsion aerosol formulation comprises, for example, ethanol, surfactant, water and propellant. Another example of an emulsion aerosol formulation comprises, for example, vegetable oil, glyceryl monostearate and propane.

### Kits

Disclosed herein, in some embodiments, are kits for using the compositions, the pharmaceutical compositions, or the cells described herein. In some embodiments, the kits disclosed herein is used to treat a disease or disorder in a subject; or select a subject for treatment and/or monitor a treatment disclosed herein. In some embodiments, the kit comprises the pharmaceutical compositions, or the cells described herein, which can be used to perform the methods described herein. Kits comprise an assemblage of materials or components, including at least one of the compositions. Thus, in some embodiments the kit contains a composition including of the pharmaceutical composition, for the treatment of the disease or disorder described herein.

In some instances, the kits described herein comprise components for selecting for a homogenous population of the engineered extracellular vesicles. In some instances, the kits described herein comprise components for selecting for a heterogenous population of the engineered extracellular vesicles In some embodiments, the kit comprises the components for assaying the number of units of the immune checkpoint moiety expressed on the surface of the engineered extracellular vesicle. In some embodiments, the kit comprises components for performing assays such as enzyme-linked immunosorbent assay (ELISA), single-molecular array (Simoa), PCR, and qPCR. The exact nature of the components configured in the kit depends on its intended purpose. For example, some embodiments are configured for the purpose of treating a disease or condition disclosed herein (e.g., autoimmune disease) in a subject. In some embodiments, the kit is configured particularly for the purpose of treating mammalian subjects. In some embodiments, the kit is configured particularly for the purpose of treating human subjects.

Instructions for use may be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

Disclosed herein, in some embodiments are methods of treating a disease or a disorder in a subject, comprising administrating a therapeutic effective amount of the compositions or pharmaceutical compositions described herein to the subject. In some embodiments, the disease or disorder comprises graft-versus-host disease (GVHD). GVHD may be a multisystem disorder which can resemble an autoimmune or immunologic disorder, and may be mediated by alloreactive and autoreactive donor T cells and B cells.

In some embodiments, GVHD comprises ocular GVHD. Ocular GVHD may involve both cell-mediated and humoral immunity and may lead to infiltration and inflammation of the lacrimal gland, conjunctiva and ocular surface. Ocular GVHD may involve infiltration of mononuclear cells around medium-size ducts in lacrimal gland acinar tissue leads to loss of acinar lobules replaced by fibrosis in patients with ocular chronic GVHD. The inflammation may eventually cause a decrease in the density of conjunctival goblet cells as well as scarring of the lacrimal gland and conjunctiva. A history of allogenic bone marrow transplant may present in patients with this disease. Affected patients often complain of dry eye symptoms, such as chronic redness, eye pain, photophobia, foreign body sensation, and decreased vision. If left untreated, ocular GVHD may lead to extensive vision loss and possible blindness. Therefore, there is a need to treat ocular GVHD.

In some embodiments, the exosomes used for treating ocular GVHD comprise any of the exosomes described herein (e.g., exosomes comprising any one or more of a passenger (e.g., passenger protein, passenger peptide, or passenger molecule)). In some embodiments, the exosomes used for treating ocular GVHD are loaded with PD-L1 (e.g., an extracellular domain of PD-L1) & CD200 (e.g., an extracellular domain of CD200). In some embodiments, the exosomes used for treating ocular GVHD are loaded with IL-2. In some embodiments, the exosomes used for treating ocular GVHD are loaded with any one or more of the passengers (e.g., there is a heterologous mix of payloads). In some embodiments, the exosomes used for treating ocular GVHD are loaded with different passengers. PD-L1 may be useful in suppressing different populations of T cells and may even induce regulatory T cell activation. CD200 may be an inhibitor of inflammatory macrophage/microglia activity. Thus, presentation of CD200 and PD-L1 may allow for a useful mechanism for engaging T cells and macrophages.

In some embodiments, administration of a pharmaceutical composition comprising the exosomes disclosed herein (e.g., exosomes loaded with PD-L1 & CD200, IL-2, or a combination thereof) is used for In some embodiments, administration of a pharmaceutical composition results in an the treatment of dry eyes.

In some embodiments, administration of a pharmaceutical composition comprising the exosomes disclosed herein is used for the treating ocular GVHD. Administration of the pharmaceutical composition may result in increased tear production. In some embodiments, administration of the pharmaceutical composition results in improved Schirmer test results. In some embodiments, Schirmer test results improve by at least 5%, at least 10%, at least 15%, at least 20%, at least 25%, at least 30%, at least 40%, at least 45%, or at least 50%. In some embodiments, an improvement in Schrimer test results comprises a normal test result (e.g., more than 10 mm of moisture on filter paper after 5 minutes) when the subject previously comprised an abnormal Schirmer test result.

In some embodiments, administration of the pharmaceutical composition may result in improvement of ocular GVHD manifestations, such as, for example, new-onset dryness, grittiness, or eye pain. In some embodiments, clinical manifestations of ocular GVHD include irritation burning, photophobia, redness, blurred vision, foreign body sensation, cicatricial conjunctivitis, keratoconjunctivitis sicca, confluent areas of punctate keratopathy, corneal ulcerations, scleritis, periorbital hyperpigmentation, blepharitis (eye lid erythema with edema and telangiectasia of lid margin), or a combination thereof. In some embodiments, administration of the pharmaceutical composition may result in improvement of one or more ocular GVHD manifestation by about 5% to about 90%. In some embodiments, administration of the pharmaceutical composition may result in improvement of one or more ocular GVHD manifestation by about 5% to about 10%, about 5% to about 15%, about 5% to about 20%, about 5% to about 25%, about 5% to about 30%, about 5% to about 40%, about 5% to about 50%, about 5% to about 60%, about 5% to about 70%, about 5% to about 80%, about 5% to about 90%, about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 20% to about 25%, about 20% to about 30%, about 20% to about 40%, about 20% to about 50%, about 20% to about 60%, about 20% to about 70%, about 20% to about 80%, about 20% to about 90%, about 25% to about 30%, about 25% to about 40%, about 25% to about 50%, about 25% to about 60%, about 25% to about 70%, about 25% to about 80%, about 25% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some embodiments, administration of the pharmaceutical composition may result in improvement of one or more ocular GVHD manifestation by about 5%, about 10%, about 15%, about 20%, about 25%, about 30%, about 40%, about 50%, about 60%, about 70%, about 80%, or about 90%. In some embodiments, administration of the pharmaceutical composition may result in improvement of one or more ocular GVHD manifestation by at least about 5%, about 10%, about 15%, about 20%, about 25%, about 30%, about 40%, about 50%, about 60%, about 70%, about 80%, or about 90%.

In some embodiments, administration of the pharmaceutical composition results in improved anterior slit lamp examination results. In some embodiments, administration of the pharmaceutical composition results in improved Schirmer test results and anterior slit lamp examination results.

In some embodiments, administration of the pharmaceutical composition results in an improved scoring eye involvement per 2014 NIH scoring criteria. In some embodiments, the improved scoring eye involvement criteria is improved by 1, 2, or 3.

In some embodiments, administration of the pharmaceutical composition results in less need for concurrent ocular GVHD treatment (e.g., artificial tears, lubricants, gels, scleral lenses, other topical immunosuppressive treatments (e.g., cyclosporin A, topical tacrolimus, topical corticosteroids, etc.), punctual plus, punctual cautery, systemic steroids, topical anti-inflammatories, etc). In some embodiments, administration of the pharmaceutical composition may result in less concurrent ocular GVHD treatment(s) by about 5% to about 90%. In some embodiments, administration of the pharmaceutical composition may result in less concurrent ocular GVHD treatment(s) by about 5% to about 10%, about 5% to about 15%, about 5% to about 20%, about 5% to about 25%, about 5% to about 30%, about 5% to about 40%, about 5% to about 50%, about 5% to about 60%, about 5% to about 70%, about 5% to about 80%, about 5% to about 90%, about 10% to about 15%, about 10% to about 20%, about 10% to about 25%, about 10% to about 30%, about 10% to about 40%, about 10% to about 50%, about 10% to about 60%, about 10% to about 70%, about 10% to about 80%, about 10% to about 90%, about 15% to about 20%, about 15% to about 25%, about 15% to about 30%, about 15% to about 40%, about 15% to about 50%, about 15% to about 60%, about 15% to about 70%, about 15% to about 80%, about 15% to about 90%, about 20% to about 25%, about 20% to about 30%, about 20% to about 40%, about 20% to about 50%, about 20% to about 60%, about 20% to about 70%, about 20% to about 80%, about 20% to about 90%, about 25% to about 30%, about 25% to about 40%, about 25% to about 50%, about 25% to about 60%, about 25% to about 70%, about 25% to about 80%, about 25% to about 90%, about 30% to about 40%, about 30% to about 50%, about 30% to about 60%, about 30% to about 70%, about 30% to about 80%, about 30% to about 90%, about 40% to about 50%, about 40% to about 60%, about 40% to about 70%, about 40% to about 80%, about 40% to about 90%, about 50% to about 60%, about 50% to about 70%, about 50% to about 80%, about 50% to about 90%, about 60% to about 70%, about 60% to about 80%, about 60% to about 90%, about 70% to about 80%, about 70% to about 90%, or about 80% to about 90%. In some embodiments, administration of the pharmaceutical composition may result in less concurrent ocular GVHD treatment(s) by about 5%, about 10%, about 15%, about 20%, about 25%, about 3000, about 40%, about 50%, about 60%, about 70%, about 80%, or about 90%. In some embodiments, administration of the pharmaceutical composition may result in less concurrent ocular GVHD treatment(s) by at least about 5%, about 10%, about 15%, about 20%, about 25%, about 30%, about 40%, about 50%, about 60%, about 70%, about 80%, or about 90%.

In some embodiments, the pharmaceutical composition is a topical formulation. In some embodiments, the pharmaceutical composition is a topical formulation for the eye. In some embodiments, treating ocular GVHD comprises administering the pharmaceutical composition topically (e.g., onto the eye or eyes). In some embodiments, the pharmaceutical composition is an intravitreal formulation. In some embodiments, treating ocular GVHD comprises administering the pharmaceutical composition intravitreally. While preferred embodiments of the present invention have been shown and described herein, it can be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions can now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary. While preferred embodiments of the present invention have been shown and described herein, it can be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions can now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/of” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to ±10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “individual,” “patient,” or “subject” are used interchangeably. None of the terms require or are limited to situation characterized by the supervision (e.g., constant or intermittent) of a health care worker (e.g., a doctor, a registered nurse, a nurse practitioner, a physician's assistant, an orderly, or a hospice worker).

The terms “expression” or “expressing” refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which can be located upstream or downstream of the coding sequence. The term “gene” is to be interpreted broadly, and can encompass mRNA, cDNA, cRNA and genomic DNA forms of a gene. In some uses, the term “gene” encompasses the transcribed sequences, including 5′ and 3′ untranslated regions (5′-UTR and 3′-UTR), exons and introns. In some genes, the transcribed region can contain “open reading frames” that encode polypeptides. In some uses of the term, a “gene” comprises only the coding sequences (e.g., an “open reading frame” or “coding region”) necessary for encoding a polypeptide. In some aspects, genes do not encode a polypeptide, for example, ribosomal RNA genes (rRNA) and transfer RNA (tRNA) genes. In some aspects, the term “gene” includes not only the transcribed sequences, but in addition, also includes non-transcribed regions including upstream and downstream regulatory regions, enhancers and promoters. The term “gene” can encompass mRNA, cDNA and genomic forms of a gene.

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g., altered backbone, sugar, or nucleobase). Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

As used herein, the terms “polypeptide,” “peptide” and “protein” can be used interchangeably herein in reference to a polymer of amino acid residues. A protein can refer to a full-length polypeptide as translated from a coding open reading frame, or as processed to its mature form, while a polypeptide or peptide can refer to a degradation fragment or a processing fragment of a protein that nonetheless uniquely or identifiably maps to a particular protein. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

As used herein, the terms “fragment,” or equivalent terms can refer to a portion of a protein that has less than the full length of the protein and optionally maintains the function of the protein. Further, when the portion of the protein is blasted against the protein, the portion of the protein sequence can align, for example, at least with 80% identity to a part of the protein sequence.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g., thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g., the EMBOSS Needle aligner available at www.ebi.ac.uk/Tools/psa/emboss_needle/nucleotide.html, optionally with default settings), the BLAST algorithm. Optimal alignment can be assessed using any suitable parameters of a chosen algorithm, including default parameters.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are commonly known. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “in vivo” can be used to describe an event that takes place in a subject's body.

As used herein, the term “ex vivo” can be used to describe an event that takes place outside of a subject's body. An “ex vivo” assay cannot be performed on a subject. Rather, it can be performed upon a sample separate from a subject. Ex vivo can be used to describe an event occurring in an intact cell outside a subject's body.

As used herein, the term “in vitro” can be used to describe an event that takes places contained in a container for holding laboratory reagent such that it is separated from the living biological source organism from which the material is obtained. In vitro assays can encompass cell-based assays in which cells alive or dead are employed. In vitro assays can also encompass a cell-free assay in which no intact cells are employed.

“Treating” or “treatment” can refer to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. A therapeutic benefit can refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject can still be afflicted with the underlying disorder. A prophylactic effect can include delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease cannot have been made.

The term “effective amount” and “therapeutically effective amount,” as used interchangeably herein, generally refer to the quantity of a composition, for example a composition comprising immune cells such as lymphocytes (e.g., T lymphocytes and/or NK cells) comprising a system of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio.

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

U.S. Pat. Nos. 9,547,003, 10,247,727, and 10,527,609, are all incorporated by reference in their entirety herein.

### EXAMPLES

### Example 1: Corneal Transplant in Mice

Murine orthotopic corneal allo-transplants are employed using 1B6 cornea donors and MHC-mismatched B ALB/c recipients, which typically result in graft rejection rates of >80% Treatment with engineered exosomes) (e.g., exosomes described herein or exosomes loaded by the methods disclosed herein (e.g., exosomes with affinity receiver(s)/tag(s) (e.g., engineered exosomes loaded with PD-L1 & CD200) are tested at different doses and time points (days 3-9) after transplantation. Corneal graft survival is assessed weekly by clinical corneal clarity score (examples of such measuring may be shown in PCT/US2019/018554, which is herein incorporated in its entirety). The percent survival (e.g., of the corneal allografts treated with loaded exosomes) in relation to days post-transplant is measured. The effect of treatment with exosome(s) is measured by B6-BALB/c MHC-mismatched graft rejection post orthotopic corneal allograft.

The effect of engineered exosomes on Allogenic Corneal Transplant Survival C57BL/6->BALB/c is studied. Post orthotopic corneal allograft treatment with the engineered exosomes delays of B6-BALB/c MHC-mismatched graft rejection. The study is conducted and analyzed using varying doses of engineered exosomes.

The dose response of treatment with the varying doses of exosomes will be measured in relation to corneal allograft survival. The dose response will demonstrate that post-transplant exosomes(s) treatment promotes corneal allograft survival.

Animals treated with doses of engineered exosomes (e.g., in doses of g of loaded exosome/kg or mg of engineered exosome/kg at multiple time-points (ex. Days 6, 7, +/−9) are used to determine the rejection of corneal transplant (“CT”). The doses of engineered exosome(s) will delay the rejection of CT.

Furthermore, employing escalating engineered exosome doses (e.g., in the range of g of loaded exosome/kg or mg of engineered exosome/kg) corroborate the timing and/or correlate with increasing, significant corneal graft survival. Decreased MLR reactivity against donor 136 antigens is observed in engineered exosome treated recipients, consistent with deletion of graft rejecting allo-reactive T cells.

A dramatic effect on neovascularization in engineered exosome treated pre-vascularized high-risk CT recipients will be observed with the use of corneal transplants and an analysis of neo-vascularization after transplant of MHC-mismatched corneas in untreated and engineered exosome treated recipients. Photographic and slit lamp analyses will demonstrate markedly diminished overall number, size and location of vessels in exosome treated CT recipients. Within engineered exosome treated mice, long-term acceptors—but not rejectors—will demonstrated minimal/no corneal neo-vascularization. In total, the findings will illustrate that engineered exosomes can effectively and significantly prolong CT

The above data will indicate that engineered exosomes inhibits both graft rejecting T cells and corneal neo vascularization and that the latter involves reduction of angiogenic signals from infiltrating hematopoietic as well as parenchymal cells. These findings will suggest that loaded exosomes can provide a clinically effective treatment to markedly improve corneal allograft survival.

As noted above, the overall strategy of combining deletion of anti-graft rejecting T cells, inhibition of neovascularization and local expansion of Treg cells is particularly attractive because it is possible to exploit the ability of engineered exosome treatment to spare Tregs and therefore promote the opportunity to target and expand this population. The research will demonstrate that systemic administration of engineered exosomes results in marked Treg expansion Following corneal allotransplant and administration of post-transplant loaded exosomes, introduction of the present two-pathway strategy effectively expands Treg cells. In particular, Treg expansion in peripheral blood after engineered exosome treatment following B6 corneal allografts in BALB/c recipients will occur. Specifically, TL1A-Ig+IL-2 will induce Treg expansion following post-corneal transplant administration of loaded exosomes. Recipient mice will be injected engineered exosomes beginning D.10 post-CT. Mice are bled on D.16 and stained for CD4 and FoxP3.

The techniques herein provide a method of use in which a strategy to limit/e.g., restrict the expansion of regulatory T cells (CD4⁺FoxP3⁺ T cells) is implemented within the ocular compartment Periorbital delivery of these proteins will result in dramatic and local Treg expansion within the ocular adnexa and draining cervical lymph nodes. In total, this will enable the unique opportunity to develop and employ a combinatorial mechanism (Treg cells+loaded exosomes) for the establishment of allograft tolerance to maintain permanent corneal graft survival. An experiment is performed in which the above strategy is used to expand Tregs after engineered exosome treatment and although the final treatment with IL-2 is 16 days after transplant, the survival of the allograft is significantly prolonged.

The techniques herein further provide a strategy to expand Tregs by administering a “two pathway” strategy via local injection. Importantly, marked elevation of Treg cells in the conjunctiva is also observed when the strategy is employed systemically. Tregs are in fact elevated as well in the conjunctiva. However, when the same doses of engineered exosomes are administered locally and systemically, greater expansion of Tregs in the conjunctiva is observed via the local delivery

If the engineered exosomes and IL-2 are delivered unilaterally, it increases the Treg percentage dn numbers in the draining cervical lymph nodes—and there is initially almost no increase in the contra-lateral lymph nodes, indicating that local delivery results in specific expansion only in the ipsilateral, i.e. side of injection (ipsilateral). In particular, local administration of engineered exosomes+IL-2 increases Treg cells in the ipsilateral—but not—contralateral draining (cervical) lymph nodes.

Furthermore, examination of distal nodes (ex. Inguinal) demonstrates that local periorbital injection will not affect Treg percentage and numbers in these non-draining/distal nodes. In particular, giving locally elevated cervical but not inguinal, while giving locally vs systemically will not increase Tregs in inguinal nodes. In fact, injection of greater amounts of engineered exosomes+IL-2 locally fails to affect the distal nodes compared to lower levels of systemically administered compounds.

In total, the techniques herein provide that use of current anti-TNFRSF25 reagents—including evolving compounds following the use of engineered exosomes or independently provides a powerful combinatorial strategy of deletion and expansion resulting in a balance of Teffector/Tregulatory cells which will promote long-term survival of corneal allografts.

The deletion/expansion strategy developed herein can be employed to treat a number of ocular inflammatory disorders including dry eye, uveitis, scleritis and mucous membrane pemphigoid as well as posterior segment diseases.

### Example 2: Ocular Graft-Versus-Host Disease (GVHD)

While not wishing to be bound by any particular theory, the loaded exosomes provided herein (e.g., exosomes comprising the affinity receiver(s)/tag(s) disclosed herein (e.g., exosomes loaded with PD-L1 & CD200) will interfere with the functional activities of both infiltrating inflammatory as well as ocular parenchymal cells, such as cornea/conjunctiva epithelial cells, keratocytes and conjunctival stromal elements. Loaded exosomes will inhibit molecules that promote transcription of, e.g., inflammatory molecules IL-1β, TNFα, IL-6, IL-17, INFγ, CCL2, CXCL10, IL-4 and IL-10, associated with ophthalmologic inflammation, in particular keratoconjunctivitis sicca. The loaded exosomes also target the NFKβ pathway associated with induction of inflammatory proteins.

A novel pre-clinical MHC-matched allogeneic hematopoietic stem cell transplantation HSCT model (“MUD”—Matched Unrelated Donor) is developed that results in systemic and ocular GVHD with onset kinetics similar to that observed in patients using the procedures set forth in US20190247302A1, which is hereby incorporated in its entirety herein. C3H SW (H-2b, Ly9.1+) mice are transplanted with B6 (H-2b, Ly9.1−) T-cell depleted bone marrow (TCD-BM) and T cells. First, only mice receiving TCD-BM+ T cells undergo weight loss and began exhibiting clinical signs of GVHD ^{~}3 wks post-HSCT. Second, the mice also develop ocular surface disease evidenced by progression of ocular surface damage characterized by increased lid margin swelling, conjunctiva inflammation and lacrimal gland fibrosis that is associated with poor tear production, corneal staining and ulceration by week 6 after transplantation Ocular GVHD occurs in >60% of patients that undergo allogeneic HSCT and similar to our pre-clinical model, is also characterized by dry eye, meibomian gland dysfunction, conjunctiva damage, punctate keratopathy, corneal ulceration and perforation.

Histological analyses of the ocular adnexa of these animals demonstrate that only mice that developed systemic and ocular GVHD exhibited corneal thickening and epithelial irregularity, as well as dense inflammatory cell infiltrates. Significantly, in contrast to the CD8>CD4 systemic GVHD immune phenotype, the CD4>CD8 ratio immune phenotype in the ocular compartment is distinct. Furthermore, the detection of IFN-γ and TNF-α, suggests that Th1 allo-rx effector cells and M1 inflammatory mΦ are involved in ocular GVHD. Thymic injury in these mice suggests that the failure of intrathymic central tolerance may result in the involvement of donor and/or host pathogenic T cells with self-rx to ocular antigens. Based on the results, it is believed that both allo-rx donor T cells and/or self-rx T cells infiltrate the ocular surface and orchestrate the recruitment of inflammatory M1 mΦ that contribute to ocular damage

Intra-vital time-lapsed fluorescence microscopy is used to non-invasively monitor the tempo of EGFP+ transplanted donor T cells in the eye. C3H.SW mice are transplanted using B6-EGFP (1-12b) donors and EGFP expression is correlated with systemic and ocular GVHD onset and progression. The data demonstrates that recruitment of EGFP+ cells into the ocular adnexa begins between 3-4 weeks after transplantation, and inflammation of the lid margin appears to be a very sensitive marker of onset of disease. To address the requirement for CD4 and CD8 in the induction of ocular GVHD, transplants are performed using purified CD4 and CD8 subsets obtained from B6 donor mice. In contrast to control mice that received TCD-BM, mice that receive both T cells developed systemic and ocular GVHD However, the development of ocular GVHD is clinically more significant in mice that receive CD4 T cells only in comparison to CD8 T cells transplanted mice, suggesting that CD4 T cell subset may be more relevant in the development of ocular GVHD.

Furthermore, chemokine and cytokine RNA findings from ocular tissue in mice with GVHD find, a) T cell chemo-attractant, CXCL10 and CXCR3 expression b) cytokines reflective of M1 inflammatory mΦ and Th1 effector cells. The presence of these effector molecules is demonstrated at the ocular surface of patients with ocular GVHD. To test the hypothesis that a potential mechanism of inflammatory T cell recruitment into the ocular adnexa of mice with GVHD may be dependent on the CXCL10/CXCR3 axis, transplants using T cells from B6-CXCR3 knock out mice are performed. The data demonstrates that mice transplanted with CXCR3 knock out T cells develope less ocular GVHD in contrast to mice that received wild type T cells. The data suggests that inhibiting the recruitment of ocular GVHD T cells using anti-chemokine receptors compounds may be a strategic approach to prevent or treat ocular GVHD, especially if it can be delivered locally.

Studies with epigenetic regulators are conducted to investigate if such compounds alter the phenotype and/or function of inflammatory cells recruited to this compartment. To test the role of bromodomain inhibition in ocular inflammation, an in vivo model of corneal inflammation induced by LPS is developed. Loaded exosomes are administered systemically by intraperitoneal injection beginning ^{~}15 hours prior to the induction of LPS keratitis.

This model represents clinically relevant ophthalmic pathological damage cause by bacterial infections. Corneal infiltrates are collected from multiple corneas and analyzed by flow cytometry for the intracellular production of TNFα in CD11b+(macrophage) ocular infiltrating cells. The data demonstrates that, similar to the results of in vitro experiments, loaded exosomes effectively reduce the production of pro-inflammatory cytokines in vivo, specifically in the eye. This is demonstrated by flow cytometric analysis gated on CD11b+ corneal infiltrate and flow cytometric protein staining and cell numbers in corneal infiltrate.

The ability of loaded exosomes to regulate several cytokines is examined using a macrophage cell line (RAW cells) after stimulating these cells with LPS to induce inflammatory molecules and first demonstrating significant down regulation of TNFα RNA. In vitro data demonstrates that, following stimulation with lipopolysaccharide (LPS—present on the cell walls of bacteria) to induce inflammatory molecules, significant downregulation of TNFα RNA is observed. The production of inflammatory cytokines TNFα, IL-6 and 11-12 by LPS-stimulated primary CD11b+(predominantly macrophages and dendritic cells) spleen cells is downregulated by loaded exosomes Studies then examine protein production and confirm that TNFα is reduced by treating RAW cells in vitro with multiple loaded exosomes (e.g., multiple loaded exosomes comprising multiple different loads). The production of TNFα, IL-6 and IL-12 by LPS stimulates primary CD11b+ spleen cells (predominantly macrophages and dendritic cells) is markedly down regulated by loaded exosomes as demonstrated by intracellular cytokine staining. Similar results are detected with RAW cells using additional loaded exosomes.

To assess the capacity of loaded exosomes to inhibit ocular inflammation, an in vivo model of corneal inflammation induced by LPS is utilized that represents clinically relevant ophthalmic inflammatory response. Loaded exosomes are first administered systemically by intraperitoneal injection beginning ^{~}15 hours prior to the induction of LPS keratitis Infiltrates are collected from multiple corneas and analyzed by flow cytometry for the intracellular production of TNFα in CD1 b+(macrophage) ocular infiltrating cells. Similar to the in vitro experiments, loaded exosomes reduce the production of a pro-inflammatory cytokines in vivo, specifically in the ocular compartment.

Next, to address whether local administration of loaded exosomes could be used to regulate ocular inflammatory responses, an ophthalmic formulation loaded exosomes are developed.

### a New Ophthalmic Formulation is Developed.

The loaded exosomes are diluted at higher concentrations when formulated with DMSO, polysorbate (Tween-80), preservative free 0.9% NaCl, and captisol (Captisol®), which is a polyanionic beta-cyclodextrin derivative providing. The new solution enables formulation of significantly higher concentrations of the loaded exosomes for local ophthalmic application. Using this new formulation, the concentrations of both DMSO and Tween-80 are able to be reduced (e.g., from 5% to 0.5% of each (which is tolerable to the eye)).

A formulation comprising loaded exosomes is delivered locally in the eye by subconjunctival injection (20 μg/kg/body weight) just prior and during the induction of in vivo LPS-induced keratitis. The data will demonstrate that the local ocular application of loaded exosomes ameliorates the development of corneal inflammation, as illustrated by clinically increased clarity. To confirm that a second type of loaded exosome could successfully be used in the ocular compartment to reduce inflammation, a second type of loaded exosome dissolved in the new ophthalmologic formulation was locally administered. The formulation is applied topically, (e.g., on the corneal surface 1 drop/3× per day). This topical application of the formulation comprising a second type of loaded exosome will also successfully ameliorate the inflammatory response in the cornea of LPS treated animals.

The ability of loaded exosomes to regulate production of dry eye-associated cytokines and chemokines produced by human ocular parenchymal cell populations is was examined. In vitro cells from the cornea and conjunctiva compartments of donated human eyes (Florida Lions Bank) are expanded. Primary cell lines re developed comprising either corneal keratocytes or conjunctival epithelial cells (HCEC) Following 3 hr. exposure to LPS, HCEC produced significant levels of IL-8, IL-13, TNFα, CCL2, and detectable levels of IL-6 RNA. HCEC incubated for the 3 hours with loaded exosomes demonstrate marked decreases in RNA levels of IL-8, TNFα, CCL2 and IL-6, and IL-1β was marginally reduced. Corneal keratocyte cultures are also examined for RNA production and are also regulated by loaded exosomes. The levels of IL-13, IL-6, and the chemokine CCL2 are effectively suppressed by loaded exosomes. These observations extend the potential of loaded exosomes to inhibit ocular inflammation. Additionally, differences may exist between the families of loaded exosomes such that particular loaded exosomes may be more useful under a specific set of disease conditions.

The effect of prior formulations on corneal clarity and corneal edema following LPS-induced keratitis is studied using clinical haze score, time after application of LPS+ loaded exosomes (topical); loaded exosomes, subconjunctival; solvent, subconjunctival). TLlocal administration of loaded exosomes using prior formulations demonstrates no significant effect on corneal pacification following LPS-induced keratitis.

The effect of a set of loaded exosomes of the instant disclosure (administered via subconjunctival injection) on corneal clarity and corneal edema following LPS-induced keratitis was studied using clinical haze score, time after application of LPS+loaded exosomes, solvent alone, and saline. Local subconjunctival administration of loaded exosomes prevents corneal opacification following LPS-induced keratitis.

The effect of a set of loaded exosomes of the instant disclosure (topical administration) on corneal clarity and corneal edema following LPS-induced keratitis was studied using clinical haze score, time after application of LPS+ loaded exosomes (topical), and solvent alone (subconjunctival) Topical administration of loaded exosomes prevents corneal opacification following LPS-induced keratitis.

The effect of loaded exosomes on a human ocular surface primary parenchymal cell line are studied. Cytokine/GAPDH mRNA levels are measured (ratio to no treatment) of IL-8, IL-1β, IL-6, TNFα, and CCL2 in human corneal endothelial cells are administered no treatment, three hours after exposure to LPS, and three hours after exposure to LPS and loaded exosomes. Cytokine/GAPDH mRNA levels are measured (ratio to no treatment) of IL-8, IL-10, and IL-6 in keratocytes administered no treatment, four hours after exposure to LPS, four hours after exposure to LPS and loaded exosomes, and four hours after exposure to LPS and comparator. Cytokine/GAPDH mRNA levels are measured (ratio to no treatment) of CCL2 in keratocytes administered no treatment, four hours after exposure to LPS, four hours after exposure to LPS and CCL2, and four hours after exposure to LPS and comparator. The data shows epigenetic regulation of human ocular surface primary parenchymal cell line responses to LPS-induced inflammation.

The formulation comprising loaded exosomes was delivered locally in the eye by subconjunctival injection in a series of experiments to conduct a therapeutic analysis of loaded exosomes for the treatment of ocular GVHD. The below disclosure shows loaded exosomes administration schedules for three independent experiments analyzing ocular GVHD (OGVHD). Experiment OGVHD #28 includes data from a therapeutic analysis of loaded exosomes for treatment of ocular GVHD in MUD B6->C3H.SW mice in which loaded exosomes was administered topically 2°/day and 3×/week (M/W/F). Experiment OGVHD #33 includes data from a therapeutic analysis of loaded exosomes which was administered topically 2×/day (M T W Th F) and subconjunctival every other Friday. The third experiment OGVHD #36 includes data from a therapeutic analysis of loaded exosomes for treatment which was administered topically 1×/day on W and F and subconjunctival on Mondays and Fridays

Individual eyelid scores and the overall clinical response over the course of the experiment OGVHD #28 are analyzed. Local treatment with the loaded exosomes decreased inflammation of the lid as evidenced by decreased lid edema. Similarly, the resolution of lid edema as seen using a different treatment protocol with loaded exosomes (e.g., OGVHD #33). There is an effect of the loaded exosomes being assessed, and suggest that modification of the treatment regimen may be able to increase efficacy.

The development of systemic GVHD in our MUD model of B6→C3H.SW as assessed by weight loss, clinical score, and overall survival. The progress of systemic GVHD in this mouse model, and demonstrate that the local administration of loaded exosomes does not affect the development of this GVHD.

The ocular clinical response to local application of loaded exosomes in these mice with systemic ocular GVHD is studied. In this experiment treatment is further modified to include topical and subconjunctival application. The results demonstrate the significant resolution of lid edema over the course of treatment for 6 weeks, which is initiated after GVHD onset. Additionally, individual eyelid scores are recorded throughout the duration of the experiment. The study demonstrates that ocular GVHD can be treated by local administration of engineered exosomes.

The data demonstrates that local manipulation of the ocular infiltration and regulatory pathways associated with donor and potential host resident inflammatory cells in the ocular adnexa can be targeted to efficiently prevent and treat ophthalmic GVHD. The use of engineered exosomes provides important advantages in regulating inflammation in the ocular compartment.

### Example 3: Treatment Effect on Ocular GVHD

Mice (C3H.SW, H2b, Ly9.1P) are placed in a holding device to transport them for irradiation (total body irradiation 10.5 cGy using a Gamma Cell 40 [35-40 rad/min] ~3 to 4 hours prior to transplantation) and are optionally provided antibiotic water (gentamycin, 25 mg/gallon) from day 3 to day 14 post-transplant for prophylaxis against bacterial infection. The mice are then either 1) treated with varying doses of pharmaceutical compositions comprising loaded exosomes (e.g., the engineered exosomes comprising PD-L1 and CD200) for a period of time (e.g., 1 day, 2 days, 3 days, 4 days, 5 days, 6 days, 7 days, 10 days, 14 days, 21 days, 28 days, 35 days, 42 days, 49 days, or 60 days) or 2) are controls. In most experiments, the donor cells are obtained from unmanipulated mice of various genetic backgrounds Donor B6 mice (H12b, Ly9.1) are euthanized by cervical dislocation, and tissues are harvested and processed.

Femurs and tibiae are removed from donor mice and bone marrow cells (BMCs) flushed with cold RPMI-1640 using a syringe fitted with a 26-gauge needle. Donor marrow inoculum (TCD-BM) is prepared using anti-Thy-1.2 Miltenyi MACS (San Diego, CA, USA) magnetic beads and negative selection to remove T cells, washed, and adjusted before transplant to 1 3 107/mL. To prepare donor T cells, spleen and lymph node cells are incubated on anti-sIg-coated (Millipore, Darmstadt, Germany) plastic dishes for 45 minutes at 48 C to remove B cells. Nonadherent cells are harvested, and a small aliquot was stained with anti-CD4 and anti-CD8 mAb (13D Pharmingen, San Diego, CA, USA) to determine precise percentage contributions. Cell suspensions containing donor bone marrow and T cells are adjusted in serum-free RPMI to the desired concentration (4.6 3 106/mL) for intravenous (0.5 mL) injection (2.3 3 106 T cells/mouse).

### Flow Cytometry

Lymph nodes, spleens, and peripheral blood are collected, and single-cell suspensions are prepared in PBS+2% fetal calf serum b azide. Corneas are harvested, pooled, and incubated in 13 PBS (pH 7.2-7.4) supplemented with EDTA (20 mM) for 15 minutes at 378 C. After washing, corneas are sliced into small fragments and incubated with collagenase (82 units/cornea, Sigma-Aldrich Corp., St Louis, MO, USA) for 60 minutes at 378 C. Following the incubation, the corneas are dissociated into single-cell suspension and filtered using cell strainers (13D Falcon, Franklin Lakes, NJ, USA). After one wash with staining buffer (13 PBS supplemented with 1% FBS and 0.1% sodium azide (Sigma-Aldrich Corp), cells are stained for CD4, CD8, CD62L, and CD44 (eBiosciences, San Diego, CA, USA). Fluorescence-activated cell sorting (FACS) analysis of live lymphocytes was performed using Becton Dickinson LSRII and FACS Diva software (BD, San Jose, CA, USA) and FlowJosoftware (Tree Star, Ashland, OR, USA).

### Systemic and Ocular GVHD Assessment

Systemic GVHD is assessed using two analyses. Peripheral blood (and lymph node and spleen tissue in some experiments) are harvested to assess immune phenotype characteristic of GVHD using fluorescent conjugated mAbs to analyze CD4/CD8 ratio and B cell levels (anti-CD19 mAb). Animals are also monitored for clinical changes characteristic of GVHD. The onset/presence of GVHD is monitored before the HSCT and then weekly by a modified version of a standard scoring system. This system incorporates seven clinical traits weight loss, posture, activity, fur texture, skin integrity, alopecia, and presence of diarrhea. Each trait is scored from 0 to 2, with a range from 0 to 14. Ocular GVHD is assessed using two analyses. Corneal lysates are prepared as described above to assess immune phenotype characteristic of GVHD using mAbs to analyze CD4, CD8, CD62L, and CD44. Clinical assessment is performed on all mice at baseline, and then weekly thereafter. Mice are anesthetized with isoflurane gas, and ocular surface is assessed using corneal fluorescein staining, performed by applying 3 IL of a 0.5% sodium fluorescein solution (Sigma-Aldrich Corp.) onto the eye. After washing with a balanced salt solution, the cornea is examined and photographed using an automated fluorescence microscope (Leica MZ16FA; Leica Microsystems, Wetzlar, Germany) and a cobalt blue light 2 to 3 minutes after fluorescein application. Punctate epithelial staining is scored using a previously described grading system in which the cornea is divided into five areas and a score is determined for each area, generating a clinical index with a maximum score of 15.

### Histopathology and Immunohistochemistry

Mice that received treatment (e.g., the mice are treated with varying doses of pharmaceutical compositions comprising loaded exosomes (e.g., the loaded exosomes disclosed herein and/or using the methods disclosed herein) for a period of time (e.g., 1 day, 2 days, 3 days, 4 days, 5 days, 6 days, 7 days, 10 days, 14 days, 20 days, 21 days, 28 days, 30 days, 35 days, 40 days, 42 days, 45 days, 49 days, or 60 days)) and control mice (e.g., those receiving no treatment) are euthanized at 6 to 7 weeks after bone marrow transplantation. Eyes including the upper lids, conjunctiva, and fornix as well as lacrimal glands are harvested and embedded in OCT compound (Sakura Finetek USA, Inc., Torrance, CA, USA) and sectioned on a cryostat (Leica, Wetzlar, Germany) (208C) at 8 lm thick and collected on microscope slides. Histopathology was performed with hematoxylin/eosin (H&E) staining on corneal sections in order to evaluate parameters such as corneal thickness, inflammatory cell infiltration, vascularization, and integrity of the corneal endothelium. Lacrimal glands are stained with H&E, periodic acid Schiff (PAS), and Masson's trichrome. The counting of conjunctival goblet cells is performed as follows: 8-lm sections are obtained from the superior conjunctiva of all animals. They are initially formalin fixed on the slides and then stained with PAS. Goblet cell count is performed in at least 10 sections for each eye, and the average number was used for statistical analysis

Immunohistochemistry is performed using the following protocol. The sections are fixed with 3% formaldehyde for 25 minutes, then pretreated with a blocking solution containing 0.05% Tween 20 and 3% BSA in PBS for 1 hour at room temperature to saturate nonspecific binding sites. The sections are then incubated 1 hour at room temperature with the primary antibody CD4, CD8 (T cells), CD11b (monocytes/macrophages), and Ly6G (neutrophils) from BD Pharmingen diluted 1/100 in PBS-Tween and 1% BSA. Sections are then rinsed for 10 minutes in PBS-Tween and incubated with 1:2000 goat anti-rat Alexa Fluor 594 (Invitrogen, Carlsbad, CA, USA) for 1 hour at room temperature. The sections are washed for 10 minutes in PBS Tween (33) and 10 minutes in PBS (13), coverslipped with Vectashield with 40, 6-diamidino-2-phenylindole (DAPI; Vector Laboratory, Inc., Burlingame, CA, USA), and photographed in a Zeiss universal microscope (Carl Zeiss, Oberkochen, Germany) equipped for incident-light fluorescence.

### In Vivo Ocular Fluorescent Stereomicroscopy

Recipients of EGFP-labeled cell populations are evaluated at weekly time points using intravital fluorescent microscopy (Leica MZ16FA, EGFP 2 filter [excitation 480/40 nm, barrier 510 nm]), allowing measurement of EGFP expression in the cornea, which is quantified as mean green intensity (MGI). Photographs of the cornea are taken using the biomicroscope in a standardized fashion for all eyes. The image is analyzed using ImagePro software (Media Cybernetics, Rockville, MD, USA), and MGI is calculated for each cornea.

### Quantitative RT/PCR

Total RNA is isolated from corneas using the RNeasy kit (Qiagen, Valencia, CA, USA); then cDNA is generated with the Maxima First Strand cDNA Synthesis Kit (Fermentas, Thermo Fisher, Grand Island, NY, USA). Gene expression is measured by real-time quantitative PCR (qPCR) using various primers (see below). Quantitative PCR is performed using iQSYBR Green Supermix (Bio-Rad, Hercules, CA, USA) on a Roche Light Cycler real-time PCR instrument (Roche, Indianapolis, IN, USA). Relative gene expression is calculated using the DDCt method, with gene expression normalized to glyceraldehyde 3-phosphate dehydrogenase (GAPDH) expression. Each treatment is represented as relative expression (i.e., fold expression over reference group), where the control sample served as the reference with a set value of 1. Primer pairs for cytokines were generated by IDT (Coralville, IA, USA)
GAPDH, Fwd: AGGTCGGTGTGAACGGATTTG (SEQ ID NO: 75),
Rev: TGTAGACCATGTAGTTGAGGTCA (SEQ ID NO. 76)
IFNc, Fwd: ATGAACGCTACACACTGCATC (SEQ ID NO: 77),
Rev: CCATCCTTTTGCCAGTTCCTC (SEQ ID NO: 78)
TNFa, Fwd: CTGAACTTCGGGGTGATCGG (SEQ ID NO: 79),
Rev: GGCTTGTCACTCGAATTTTGAGA (SEQ ID NO. 80)
IL6, Fwd: TAGTCCTTCCTACCCCAATTTCC (SEQ ID NO: 81),
Rev: TTGGTCCTTAGCCACTCCTTC (SEQ ID NO: 82)

### Statistical Analysis

The unpaired t-test is used to compare between control and experimental for fluorescein staining and conjunctival goblet cell score. Both eyes are evaluated for ocular surface staining, and the eye with the worse fluorescein staining score was selected for statistical analysis. A P value of 0.05 is considered statistically significant. GraphPad Prism 5 (GraphPad Software, San Diego, CA, USA) is used for the analyses

### RESULTS

Clinical Ocular Changes in Control Recipients Compared to Recipients Receiving Pharmaceutical Compositions Comprising Loaded Exosomes Undergoing GVHD in a Major Histocompatibility Complex (MHC)-Matched, Minor Transplantation Antigen-Mismatched Allogeneic Hematopoietic Stem Cell Transplant Model

MHC-matched (H2b) C3H.SW mice (e.g., mice treated with engineered exosomes loaded with PD-L1 and CD200 and control mice) are lethally conditioned and several hours later received donor 36 BMCs replete with B6 T cells Several weeks post HSCT, animals receiving donor T cells lose weight and began to exhibit clinical signs or characteristic of GVHD including ruffled fur, hunching, and diarrhea. Animals receiving loaded exosomes do not exhibit clinical signs or characteristics (e.g., to the same extent as animals not receiving treatment). Recipients are examined for additional immunologic phenotypes characteristic of GVHD including decreased splenic cell numbers and diminished B cells. To monitor for changes in the ocular surface in recipients of HSCT, animals are anaesthetized and the corneal surface was assessed by clinical examination and fluorescein staining. Approximately 3 to 4 weeks following transplantation, increased fluorescein staining was observed only in the cornea of recipient control mice that received donor T cells, and this progressed to corneal ulcerative lesions by ~6 weeks, whereas mice receiving loaded exosomes do not exhibit increased fluorescein staining (e.g., to the same extent as control mice) Quantitative analyses for corneal staining and clinical changes demonstrate a difference in the tempo of induction between systemic and ocular GVHD in control mice as compared to mice receiving loaded exosomes

Histopathologic Damage of the Ocular Surface and Adnexa of Animals with GVHD

To characterize damage occurring in the ocular compartment of animals with GVHD, the eyes are assessed for histopathologic changes and cellular infiltrates in control mice and mice that received loaded exosomes. Histologic analyses demonstrate that only control mice develop systemic and ocular GVHD exhibited corneal thickening and epithelial irregularity, as well as dense inflammatory cell infiltrates. Immunohistochemistry revealed that in control mice, multiple mononuclear cells infiltrate the cornea as evidenced by CD11b+-stained cells as well as CD4+ and some CD8+ T cells Such features are not apparent in mice receiving loaded exosomes (e.g., to the same extent as the control mice). Control mice exhibit presence of Ly6G-staining cells supports whereas mice treated with loaded exosomes do not exhibit this feature (e.g., to the same extent as the control mice). Analyses of ocular adnexa in control mice indicates that the fornix region of the conjunctiva appeared atrophic and goblet cells were reduced in density and number. Such features are not apparent in mice receiving loaded exosomes (e.g., to the same extent as the control mice). In control mice, hematoxylin/eosin- and Masson's trichrome-stained sections of the lacrimal glands reveals periductal fibrosis and dense cellular infiltrates, which consist of predominantly macrophages (CD11b) and CD8+ T cells together with some CD4+ infiltrate. Such features are not apparent in mice receiving loaded exosomes (e.g., to the same extent as the control mice). The data indicates that similar to ocular GVHD occurring in patients who undergo HSCT, all the structures of the ocular adnexa in this preclinical model of GVHD are involved and can lead to sicca and scarring. The data also indicates that mice receiving loaded exosomes all the structures of the ocular adnexa in this preclinical model of GVHD are not involved (e.g., to the same extent as the control mice) and do not lead to sicca and scarring (e.g., to the same extent as in the control mice).

### Identification of Donor T-Cell Populations Infiltrating the Ocular Compartment in GVHD

The findings demonstrate the presence of immunologic cells recruited to the site of ocular inflammation in control mice, whereas mice receiving loaded exosomes do not exhibit these features (e.g., to the same extent as the control mice). There is a presence of lymphoid cells in the ocular compartment in control mice, but not in mice receiving engineered exosomes. Transplants are performed using T cells derived from B6-EFGP control mice and mice receiving engineered exosomes. Recipient C3H.SW control mice of transplants containing T cells from B6-EGFP donors together with marrow cells from congenic, FP CD45. 1 donors develop ulcerative lesions, and GFP+ cells are identified by intravital fluorescent microscopy of the eyes Mice receiving engineered exosomes do not exhibit these features (e.g., to the same extent as the control mice). Notably, the appearance of T cells was first identified 2 to 3 weeks post transplant and prior to corneal ulceration in control mice. Mice receiving engineered exosomes do not exhibit these features (e.g., to the same extent as the control mice).

To assess the relative T-cell subsets present, corneal lysates are prepared ~6 weeks post HSCT and cells stained for CD4 or CD8 expression. The CD4/CD8 ratio is typically inverted in the periphery of control mice undergoing GVHD and other lymphoid tissue. Mice receiving loaded exosomes do not exhibit these features (e.g., to the same extent as the control mice).

An experiment is performed using donor T cells from B6-CXCR6/EGFP knock-in mice (i.e., Bonzo/STRL33), which express EGFP regulated by the CXCR6 promoter. Notably, high expression of EGFP activated T-cell populations exhibit high expression of EGFP. Importantly. C3H.SW control recipients of B6-CXCR6% EGFP T cells lost weight and exhibited clinical changes of systemic GVHD and also develop ocular GVHD characterized by ulceration and infiltrate of the cornea. Recipients receiving engineered exosomes do not exhibit these features (e.g., to the same extent as the control recipients). Moreover, ocular suspensions of control recipients demonstrate the presence of GFPb. Ly9.1 CD4 and CD8 T cells, confirming the presence of donor T cells in these recipients' eyes. Recipients receiving loaded exosomes do not exhibit these features (e.g., to the same extent as the control recipients). In summary, the results demonstrate that transplanted donor T cells populate the ocular compartment in control recipients with GVHD, whereas mice receiving engineered exosomes do not exhibit these features (e.g., to the same extent as the control mice). The ocular infiltrate in control recipients also contains cells in the GFP fraction demonstrating the presence of CD4 and CD8 T cells. Recipients receiving loaded exosomes do not exhibit these features (e.g., to the same extent as the control recipients). These cells are Ly9.1 and therefore presumably are derived from neither transplanted donor T cells (EGFP+) nor the recipient (Ly9.1+) To begin to characterize the inflammatory milieu associated with the development of ocular GVHD, RNA was prepared from ocular lysates obtained from C3H.SW control recipients and recipients receiving loaded exosomes of B6 T cell-depleted bone marrow alone or B6 T cell-replete transplants. The data illustrates that IFNc, TNFa, and IL-6 message are significantly elevated in control mice with ocular GVHD. Recipients receiving loaded exosomes do not exhibit these features (e.g., to the same extent as the control recipients). In summary, the experiments illustrate that following allogeneic HSCT into MHC-matched allogeneic control recipients, multiple populations of donor T cells are present in the ocular compartment that appear to be active and derived from both donor-transplanted T cells and donor bone marrow. Recipients receiving loaded exosomes do not exhibit these features (e.g., to the same extent as the control recipients). Based on the use of the Bonzo/STRL33 donors, some infiltrating cells appear to be activated in control recipients. Donor T cells obtained from a B6-CD90.1 (Thy1.1+) congenic donor in an independent transplant experiment. The presence of Thy1.1b CD62Llo CD44hi CD4b T cells (as well as CD84+) in the corneal lysates of these recipients confirm that activated donor T cells were present in the ocular compartment of control recipients. Recipients receiving loaded exosomes do not exhibit these features (e.g., to the same extent as the control recipients).

### Example 4. Use of the Engineered Exosomes for the Treatment of Multiple Sclerosis (MS)

Although substantial progress has been made in drug development for relapsing-remitting MS, there remains a need for the treatment of the progressive forms of the disease. Chronic inflammation, which occurs behind a closed blood-brain barrier (BBB) with activation of microglia, may lead to damaged axonal health and impaired remyelination. As such, there is a need for therapies that promote neuroprotection and remyelination, as well as therapies which can target activated microglia in the lesions to preserve axonal health, and ideally, a therapy that could do both simultaneously. Exosomes engineered to display passenger proteins (e.g., Brain-Derived Neurotrophic Factor (BDNF) and CD200) ligand on their surface may provide a solution to these problems.

BDNF promotes tissue regeneration, but this factor has proven difficult to transport across the BBB. Complexing this factor onto the surface of an exosome may facilitate transcytosis and enable administration of therapeutic effects across the BBB (“Trojan Horse” mechanism). Further, tissue degeneration may simultaneously be slowed down by targeting activated microglia in the CNS lesions. This may be done by using CD200 ligand, which binds the CD200 receptor, CD200R, which is prominently expressed on activated microglia/macrophages.

To test these hypotheses, AIMS 1-3 were developed:

### AIM 1: Produce and Characterize Exosomes Engineered to Display CD200 and BDNF.

1.1: Produce a stable MSC-hTERT cell line genetically engineered with surface peptide binding nodes, harvest exosomes from this cell line, and confirm surface expression of the exosome peptide binding nodes.

1.2: Prime the harvested exosomes with proteins of interest: CD200 and BDNF. Three groups of exosomes are be tested in Aim 2 to evaluate the effect of varying ligand density on therapeutic activity. Group 1 consists of a 1:1 ratio of BDNF to CD200 on the exosomal surface (B1:1C), while groups 2 & 3 consist of 2:1 ratios in favor of each protein (B2:1C and B1:2C).

1.3: Characterize and quantify engineered exosomes using NanoView, flow cytometry and/or Western blotting to ensure quality and batch to batch consistency.

### AIM 2. Test Immunomodulating Activity of Engineered Exosomes in Macrophage Inhibition and Neuroprotective Assays.

2.1: Isolate human CD14+ monocytes from human PBMCs that will be differentiated into M1 macrophages using an established kit (which contains Oligodendrocyte Progenitor Cells, OPCs). These are then co-cultured with 3 groups of engineered exosomes to determine the optimal composition for M1 macrophage inhibition.

2.2: Perform neuroprotective assays using a human teratocarcinoma NT2 cell line that are differentiated into neuronal cells using retinoic acid treatment, and eventually exposed to H2O2 or glutamate to induce neuronal cell death using reactive oxygen species to mimic in vivo characteristics. This is then co-cultured with 3 groups of engineered exosomes to determine the optimal composition to preserve neuronal cell viability, which are be monitored to evaluate neuroprotection and/or regeneration using a colorimetric assay for assessing cell metabolic activity and viability (MTT) assay.

### AIM 3. Test Immunomodulating and Neuroprotective Activities of Engineered Exosomes in Chronic, and Chronic Progressive EAE Models of Multiple Sclerosis.

Evaluate the immunomodulatory, neuroprotective, and remyelination properties of optimized engineered exosomes in two EAE studies in C57BL/6 (EAE model for chronic MS) and NOD (EAE model for chronic progressive MS) murine.

Exosomes engineered to display BDNF and CD200 ligand on their surface will cross the BBB to exert therapeutic activity on CNS tissues and thereby simultaneously promote neural repair and remyelination (BDNF) and preserve axonal health, slowing down tissue degeneration by targeting activated microglia in the CNS lesions (CD200). The findings will provide insights into the feasibility of an exosome-based therapeutic for treatment of progressive forms of MS and improve understanding of the mechanisms that underlie axonal degradation and remyelination.

### Experimental Design and Methods

### AIM 1: Produce and Characterize Exosomes Engineered to Display CD200 and BDNF

Proven cell line engineering methodology that was previously validated in a HEK293 cell line will be used. CRISPR/Cas9 will be used to engineer the chromosomes of adipose-derived mesenchymal stem cells with human telomerase reverse transcriptase (MSC-hTERT; validated by ATCC) and fuse the DNA sequence of a peptide node for protein binding at the N-terminus of the native Prostaglandin F Receptor (PTGFR) gene. PTGFR is a transmembrane protein that is highly expressed on the surface of exosomes. This parental cell type has been selected due to the cell growth and phenotype stability characteristics, exosome secretion level, and for the natural immunomodulatory capacity of MSC.

Transfection will be done using a 4D Nucleofector (Lonza) using pulse code: FF 104 (optimized for human MSC). Plasmid DNA encoding the protein node/PTGFR fusion also includes a GFP tag for selection (validated by GenScript). Following nucleofection, cells will be cultured under normal conditions and expanded to approximately 20 million cells. At this time, cells are be harvested and selected for via FACS (Aria Fusion).

The MSC-hTERT parental cell line will be used to generate exosomes expressing the peptide node. Exosome producing cells will be expanded until they number 380-400 million cells, at which point they will be washed with DPBS and submerged under basal medium (ATCC) with no added supplements. Cells will be incubated for 72 h at 37° C., and the resulting conditioned media will be collected and centrifuged to remove dead cells and large debris. The solution is then subjected to Tangential Flow Filtration (TFF) through a 300 kDa MWCO filter (Repligen) to isolate and concentrate exosomes, as well as execute a buffer exchange into DPBS via diafiltration. The resulting exosomes express the peptide node on their surfaces that will couple to the tagged proteins described below. Characterization will be performed by NanoView (ExoView R100), SDS-PAGE, Western blotting, and/or flow cytometry using antibodies (BioLegend, Invitrogen) against traditional exosome tetraspanins CD9, CD63, and CD81.

NanoView is a company that specializes in exosome imaging and analysis and is the inventors' preferred method of exosome characterization. NanoView technology works similarly to ELISA in that exosome samples are exposed to chips that selectively bind CD9, CD63, and CD81 to fix individual exosomes to the surface of the chip. Chips are thoroughly washed and stained with the fluorescent antibodies of choice before being read and analyzed to supply visual readouts via ExoView software. Exosomes can be quantified using a mathematical formula relating the concentration and volume of sample applied onto the chip to the analysis' count (adjusted to prevent any overlap of tetraspanin readouts). The proteins CD200 and BDNF have been produced via Chinese Hamster Ovarian cell lines that have been genetically modified to express each protein fused to the specific complementary peptide binding tag that matches the protein node on the surface of the engineered exosomes (validated by GenScript). Proteins will be confirmed using a BCA protein assay, SDS-PAGE, and/or Western blotting. The final step of the engineered exosome product is called priming, in which the peptide-tagged proteins (BDNF and CD200) are fused to the exosomes engineered with matching nodes. This process takes up to one hour and simply requires combining 2-parts engineered exosomes and 3-parts tagged-proteins in DPBS with constant, light stirring. After priming has occurred, excess materials are washed out using diafiltration to ensure a pure final selection comprising of only exosomes fused to the proteins with 98-99% purity. This process will be used to produce 3 groups of exosomes to be tested in Aim 2. Group 1 will consist of a 1:1 ratio of BDNF to CD200 on the exosomal surface (B1:1C), while groups 2 & 3 will consist of 2:1 ratios in favor of each protein (B2:1C and B1:2C). This is easily accomplished by adjusting the amount of tagged protein added to the exosome suspension in the priming step. The rationale behind using 3 groups is to investigate how ligand surface density of each protein affects performance in functional assays of Aim 2, with the goal of Aim 2 being to select the best group to advance into Aim 3.

Quality of final engineered exosomes and batch to batch consistency will be confirmed using NanoView (ensures integrity of vesicles and confirms presence of surface molecules) and functional assays detailed in Aim 2 (each batch must demonstrate the desired biological activity).

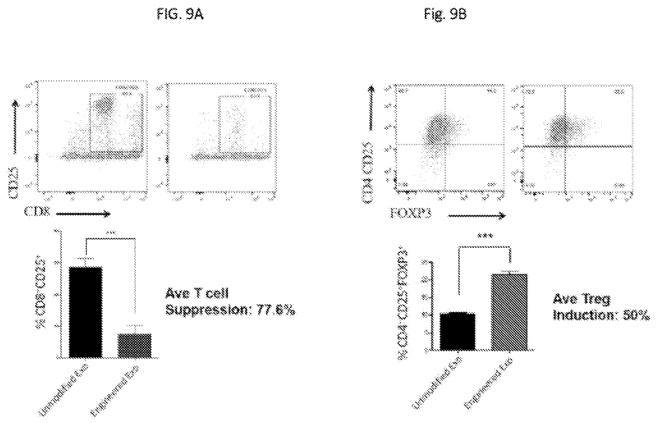

HEK293 exosomes (expressing the same peptide node) complexed with PD-L1 (fused to the same peptide tag) may be used. HEK293 exosomes complexed with PD-L1 demonstrated the increased immunomodulatory activity of engineered exosomes compared to unmodified exosomes, as shown in FIGS. 9A-9B. FIGS. 9A-9B show engineered exosomes suppress activated T cells and induce regulatory T cells (Tregs). FIG. 9A. shows that PBMCs were activated with IL-2 (500 U/mL) and co-cultured with engineered and unmodified exosomes. FIG. 9A shows that the engineered exosomes demonstrate an average T cell suppression rate of 77.6% compared to the unmodified exosomes. Following a 6-day incubation, the resulting PBMC population was analyzed via flow cytometry for CD8+/CD25+ T cells. FIG. 9B. shows that PBMCs were activated using anti-CD3+anti-CD28 Dynabeads and co-cultured with engineered and unmodified exosomes. After 3 days, the Treg population (CD4+CD25+FOXP3+) was analyzed via flow cytometry. FIG. 9B shows that the engineered exosomes demonstrate an average Treg induction rate of 50% compared to the unmodified exosomes. Paired t-tests were used to determine p values (n=3; ***p<0.001).

### AIM 2. Test Immunomodulating Activity of CD200 and BDNF Exosomes in Macrophage and Neuroprotective Assays.

### 2.1—Macrophage Inhibition Assay

To evaluate the suppressive activity of the engineered exosomes towards inflammatory acrophage/microglia, a macrophage inhibition assay will be conducted. To this end, CD14+PBMCs will be treated with GM-CSF (20 ng/mL) for 6 days to differentiate into M1 macrophage. On day 6, they will be co-cultured with LPS (10 ng/mL) in the presence of unmodified exosomes, PBS (vehicle control), or engineered exosomes (3 test groups). Culture supernatant will be harvested and stored for subsequent measurement of key inflammatory and anti-inflammatory cytokines (i.e. TNF-α, IFN-γ, IL-10, IL-17, IL-4, IL-5 and IL-6) using a flow cytometric cytokine bead array kit (BioLegend). Different concentrations of exosomes (i.e, 0.1E9, 1E9 and 3E9 total particles of exosomes, as measured by NanoView) will be used for this assay to find the optimal dose. CD200 ligand displayed on the engineered exosomes will interact with CD200R on the surface of activated macrophages and inhibit the production of proinflammatory cytokines or potentially reprogram inflammatory M1 macrophage into the M2 phenotype where anti-inflammatory cytokine production is increased. Moreover, in the CNS, CD200R is mainly expressed in microglia, but is also expressed on some subsets of T cells31. Receptor-ligand specificity ensures low probability of off-target effects, and any incidental T cell suppression may prove to be beneficial to the patient considering that T cells are also involved in PMS pathology.

Engineered exosomes that demonstrate significantly superior macrophage inhibition as quantified by expression of key inflammatory and anti-inflammatory cytokines compared to unmodified exosomes (p<0.05) will be considered for advancement into Aim 3. If any of the 3 groups fail to meet the statistical significance cutoff, they will not be considered.

In case of any difficulties differentiating CD14+ monocytes into macrophages, THP-1 (a human leukemia monocytic cell line) will be used for the experiments instead. THP-1 cells have been used extensively to study monocyte/macrophage functions, mechanisms, and signaling pathways. Using this established cell line may also decrease the variability of differentiation efficiency in experiments.

### 2.2—Neuroprotection Assay

Oxidative stress plays a crucial role in neurodegenerative processes in MS. To this end, the neuroprotective properties of engineered exosomes will be tested in a neuronal cell challenged with H₂O₂. BDNF, which is displayed on the surface of engineered exosomes, may protect neurons from H₂O₂ induced oxidative stress. BDNF may bind to tropomyosin-related kinase B receptor which is a neurotrophin receptor that is exclusively expressed in the nervous system32. Selective expression of this receptor may mitigate the risk of off-target effects from BDNF binding, and any off-target binding observed is expected to be ineffectual. Human teratocarcinoma NT2 cell line will be first differentiated into neurons and then will be challenged with 60 M H₂O₂ for 24 h in the absence or presence of unmodified exosomes, PBS (vehicle control), or engineered exosomes (3 test groups). H₂O₂-induced injuries will be assessed by MTT assay. The MTT assay is used to measure cellular metabolic activity as an indicator of cell viability, proliferation, and cytotoxicity. Different concentrations of exosomes (i.e, 0.1E9, 1E9 and 3E9 total particles of exosomes, as measured by NanoView) will be used for this assay to find the optimal dose.

A human teratocarcinoma NT2 cell line will be purchased from ATCC and used for this assay. NT2 cells will be maintained in Dulbecco's Modified Eagle's Medium (DMEM), high glucose supplemented with 10% fetal bovine serum (FBS), and 50 μg/ml gentamicin in a humidified atmosphere at 37° C. For differentiation into neurons, 2×106 cells will be plated in a 75 cm2 culture flask and treated with 10 μM retinoic acid for 2-4 weeks.

Engineered exosomes that demonstrate significantly superior neuroprotection as quantified by measurement of cellular metabolic activity via MTT assay compared to unmodified exosomes (p<0.05) will be considered for advancement into Aim 3. If any of the 3 groups fail to meet the statistical significance cutoff, they will not be considered.

Potential pitfalls and alternative approaches: If unexpected difficulties are encountered in the differentiation of NT2 cells into neurons, HT-22, an immortalized mouse hippocampal cell line, will be used. Glutamate will be used to induce damage to these cells, as HT-22 is highly sensitive to glutamate and is thus frequently used as a model system to study glutamate-induced toxicity in neuronal cells.

### AIM 3. Test Immunomodulating and Neuroprotective Activities of CD200 and BDNF Exosomes in Chronic, and Chronic Progressive EAE Models of Multiple Sclerosis.

In Aim 3, the immunomodulatory and neuroprotective properties of the engineered exosomes in vivo is evaluated. Only those exosomes which demonstrated statistically significant improvements over unmodified exosomes in Aim 2 are be chosen for testing in EAE. Human, rat, and mouse BDNF are expressed from a single gene locus, and the structure of the BDNF gene is highly conserved throughout mammals, indicating that human BDNF and murine BDNF receptors will be compatible for these studies33. CD200, CD200R, and CD200 related genes are also closely linked in humans and mice, suggesting that human CD200 will also be compatible with murine CD200R34. Two murine models of EAE will be employed: C57BL/6, a model for chronic disease, and NOD, a model for chronic PMS. Mice will be purchased from The Jackson Laboratory and will be housed in the UCSF LARC facility. Each experiment will be conducted using 10 mice per group in each of the two EAE models as follows:

EAE is induced by subcutaneous immunization of 10-week-old NOD mice or 8-week-old C57BL/6 mice into the flanks with 200 μl of an emulsion containing 150 μg of Myelin-Oligodendrocyte Glycoprotein peptide and 400 μg Mycobacterium tuberculosis H37 Ra (Difco) in incomplete Freund's adjuvant (IFA; Difco, Detroit, MI). In addition, the animals will receive 150 ng of pertussis toxin i.p. on day 0 and day 2. Unmodified exosomes (U-Exo; negative control), PBS (vehicle control), engineered exosomes (E-Exo; test group) will be tested for their effects on the C57BL/6 model of chronic EAE and in the NOD model of chronic progressive EAE. For each treatment group 10 mice per group will be used. These models will also be repeated twice, for a total of 3 times and 60 mice. The effect of escalating exosome doses (0.1E9, 1E9 and 10E9 total particles of exosomes, as measured by NanoView) administered intravenously at peak of EAE (~days 15-20) on EAE scores and CNS pathology will be investigated. Following exosome administration clinical score will be recorded daily and assigned according to Clinical signs of EAE were assessed according to the following score: 0, no signs of disease; 1, loss of tone in the tail; 2, hindlimb paresis; 3, hindlimb paralysis; 4, tetraplegia; 5, moribund.

Immunological and Histological Examination: CNS immune cell infiltration is analyzed using flow cytometry and macrophages/microglia infiltration by immunocytochemistry using the macrophage/microglia marker Iba-1. Immune cells from brain, spinal cords, spleen, and dissociated CNS tissue will be analyzed by flow cytometry for the status of macrophages, dendritic cells, B cells, and Th0, Th1, Th2, Th17, and Treg cells. Finally, brain and spinal cord sections will be scored for histology. Demyelination/remyelination will also be evaluated using immunohistochemical and Luxol Fast Blue (LFB) staining. In brief, at time of sacrifice mice will be perfused transcardially with 4% paraformaldehyde. Spinal cords will be removed and fixed in the same fixative for 24 h, washed in PBS, and then embedded in embedded in OCT in cryomolds. The fixed spinal cords will be sectioned at 8-um thickness with a cryostat and then desiccated for an hour, incubated with 10% normal goat serum or 0.5% triton in PBS for 1 h, dried and incubated with primary the respective secondary antibodies for 1 h at room temperature, washed again and mounted with DAPI for microscopic analysis.

Potential Pitfalls and Alternative Approaches: In any murine MS model, the basic parameters measuring CNS function and histology will be analyzed. Nevertheless, if any of these murine models show that E-Exo exert no significant therapeutic activity, then the efficacy of exosomes as pretreatment will be tested rather than administration at the peak of the disease. Alternatively, the dose or the frequency of exosome administration can be increased to reach desired biological effects and efficacy.

### Addressing Biological Variables in Research Designs and Analyses

The following steps are taken to address biological variables and ensure rigor and reproducibility of this research:

First, only 6-8-week-old female mice will be used as animal models. It is well established in the field of MS to use female mice as the prevalence rate is significantly higher in females. This application will not include research with human subjects. Second, experiments that include large and independent data sets will be performed in order to test them for statistical significance. The student's t-test will be used to test for significance, with p values <0.05 being considered significant at the 95% confidence level, and p values <0.01 at the 99% confidence level.

Use of Human Specimens and/or Data

### 2.1—Macrophage Inhibition Assay

CD14+PBMCs will be treated with GM-CSF (20 ng/mL) for 6 days to differentiate into M1 macrophage. On day 6, they will be co-cultured with LPS (10 ng/mL) in the presence of unmodified exosomes, PBS (vehicle control), or engineered exosomes (3 test groups). Culture supernatant will be harvested and stored for subsequent measurement of key inflammatory and anti-inflammatory cytokines (i.e., TNF-α, IFN-γ, IL-10, IL-17, IL-4, IL-5 and IL-6) using a flow cytometric cytokine bead array kit (BioLegend). Different concentrations of exosomes (i.e, 0.1E9, 1E9 and 3E9 total particles of exosomes, as measured by NanoView) will be used for this assay to find the optimal dose.

### 2.2—Neuroprotective ASSAY

A human teratocarcinoma NT2 cell line will be purchased from ATCC and used for this assay. NT2 cells will be maintained in Dulbecco's Modified Eagle's Medium (DMEM), high glucose supplemented with 10% fetal bovine serum (FBS), and 50 μg/ml gentamicin in a humidified atmosphere at 37° C. For differentiation into neurons, 2×106 cells will be plated in a 75 cm2 culture flask and treated with 10 μM retinoic acid for 2-4 weeks. Once differentiated into neurons, they will be challenged with 60 M H₂O₂ for 24 h in the absence or presence of unmodified exosomes, PBS (vehicle control), or engineered exosomes (3 test groups). H₂O₂-induced injuries will be assessed by MTT assay. The MTT assay is used to measure cellular metabolic activity as an indicator of cell viability, proliferation, and cytotoxicity. Different concentrations of exosomes (i.e, 0.1E9, 1E9 and 3E9 total particles of exosomes, as measured by NanoView) will be used for this assay to find the optimal dose.

### Example 5: Exosome Engineering Platform

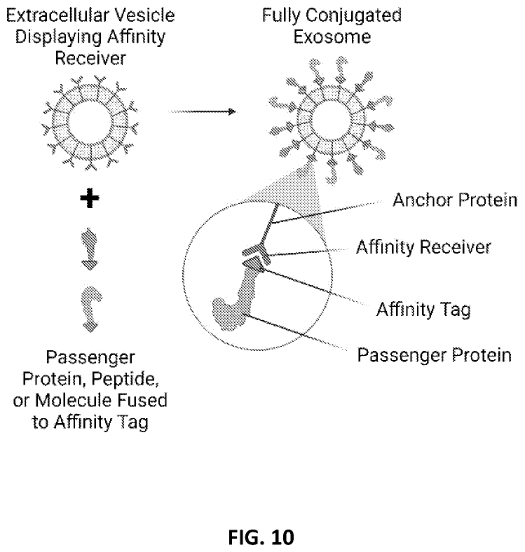

Novel methods for loading proteins, peptides, or other molecules onto the surface of extracellular vesicles (e.g., exosomes) are proposed herein. In some embodiments, at least a portion of a transmembrane moiety (e.g., an exosome-specific transmembrane moiety (e.g., CD63, CD81, or Lamp-2)) serves as an anchor protein and is fused with a peptide capable of binding an affinity tag. The protein, peptide, or molecule to be loaded (e.g., “passenger”-passenger protein, passenger peptide, or passenger molecule) may be fused with a peptide affinity tag that can bind the affinity receiver on the anchor protein. Thus, when a passenger protein having the affinity tag is mixed with an exosome carrying the anchor protein having the affinity receiver, the affinity tag may bind the affinity receiver, bringing the protein to the exosomal surface. This loading approach of using an affinity tag-receiver system may provide benefits over traditional methods of loading passenger proteins onto extracellular vesicles. For example, using the methods disclosed herein (e.g., the affinity tag-receiver system) multiple different passengers (e.g., PDL1 and CD200) may be loaded onto the surface of a single exosome. Furthermore, such a loading approach may allow for the loading of passenger proteins that are not otherwise capable of being a directly loaded onto an exosome using traditional methods, such as, for example, secreted growth factors and large proteins. FIG. 10 illustrates an exemplary affinity tag-receiver system in accordance with some embodiments disclosed herein.

### Example 6: Passenger Protein Selection for Treatment of Ocular Graft Vs Host Disease

In Ocular Graft vs Host Disease (OGvHD) pathology, donor-derived immune cells (specifically: T cells and macrophages) may cross the blood vessel barrier into the eye and drive an ocular inflammatory response, which may lead to ocular tissue damage in the lacrimal glands, meibomian glands, cornea, and conjunctiva and presents various related clinical characteristics. Donor CD4⁺ T cells and activated CD8⁺ T cells may infiltrate the periductal area of the lacrimal gland first. Then, T cell-induced tissue damage may generate a proinflammatory cascade, which may recruit macrophages, which in turn may lead to further macrophage-induced tissue damage. T cells and macrophages are believed to be the main cells involved in OGvHD pathology. Targeting both T cells and macrophages simultaneously presents a novel, promising strategy to generate effective therapeutics.

Programmed death-ligand 1 (PD-L1) may inhibit activated T cell activity when it binds to the PD-1 receptor on the surface of T cells, and CD200 may inhibit inflammatory macrophage activity when it binds to the CD200 receptor (CD200R) on the surface of macrophages. The inventors hypothesized that an extracellular vesicle (e.g., exosome) displaying both PD-L1 and CD200 could simultaneously target and suppress both T cells and inflammatory macrophages.

### Example 7: Characterization of Fully Conjugated Exosomes

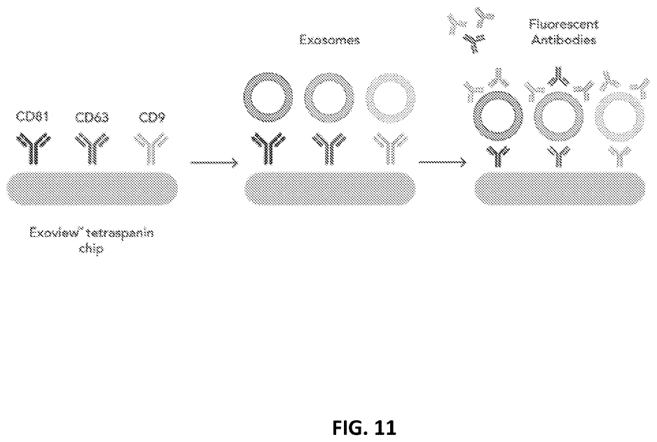

Exosomes loaded using the affinity tag-receiver system disclosed herein were characterized to confirm the presence of passenger proteins on the exosomal surface using ExoView. ExoView may be used for characterization of exosomes and other nano-scale particles. ExoView chips were coated with exosome-specific tetraspanin (CD9, CD63, and CD81) binding agents that lock exosomes to the surface of the chip. Fluorescent antibodies were used to bind proteins of interest (e.g., PD-L1, CD200, or both), and then the chips were imaged using a fluorescent microscope to visualize the particles that successfully bound the antibodies. FIG. 11 is an exemplary schematic representation of this characterization method.

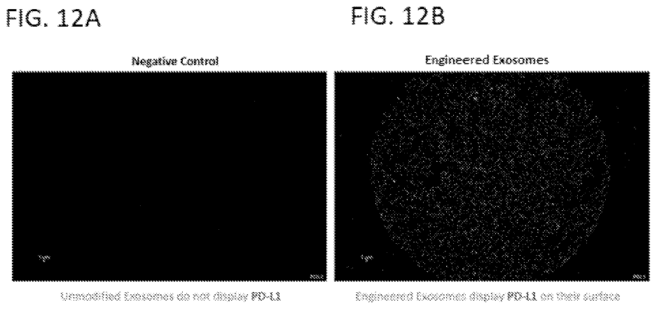

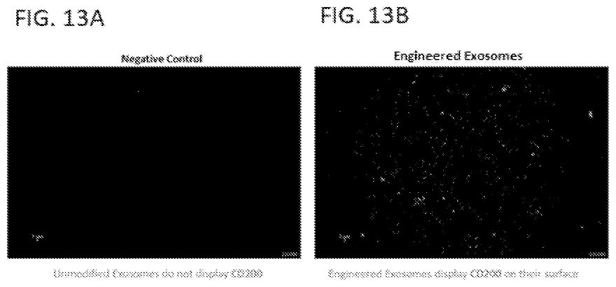

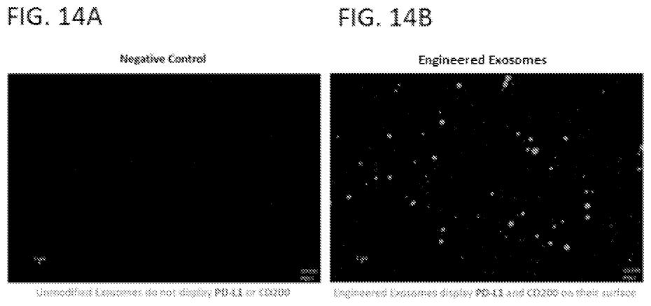

To confirm the presence of passenger proteins on the surface of engineered exosomes, one ExoView chip was loaded with fully conjugated exosomes (test group), and one chip was loaded with unmodified exosomes that had also been mixed with the same amount of passenger protein fused with the affinity tag (negative control group). FIGS. 12A-12B shows the ExoView chip when detecting PD-L1 and confirms the presence of PD-L1 on the surface of exosomes that were loaded using the affinity tag-receiver system disclosed herein. FIGS. 13A-13B shows the ExoView chip when detecting CD200 and confirms the presence of CD200 on the surface of exosomes that were loaded using the affinity tag-receiver system disclosed herein FIGS. 14A-14B shows the ExoView chip when detecting both PD-L1 and CD200 and confirms the presence of PD-L1 and CD200 on the surface of exosomes that were loaded using the affinity tag-receiver system disclosed herein. The results of this experiment demonstrate the capacity of the system to load multiple passenger proteins onto the surface of a single exosome.

### Example 8: Human Cell Assays to Measure Exosome Efficacy In Vitro

### 1. T Cell Suppression

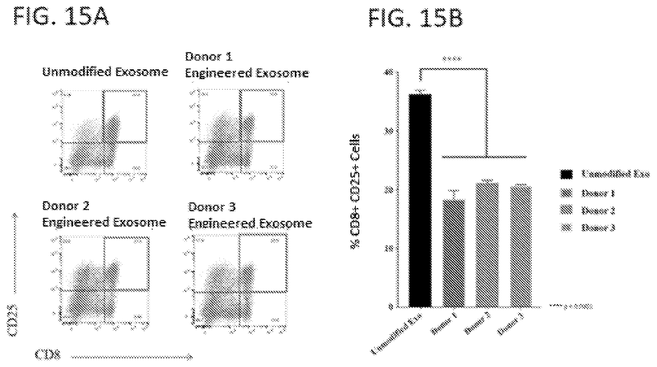

In order to evaluate the biological activity of the engineered exosomes, human cell assays were developed to measure T cell suppression using flow cytometry. Human peripheral blood mononuclear cells (PBMC) were isolated from healthy blood donors and activated to display an inflammatory phenotype (CD8⁺CD25⁺). The PBMC were co-cultured with engineered exosomes, unmodified exosomes (negative control), PBS (vehicle control), or free-floating passenger protein (protein control). Following the co-culture, PBMC were collected and analyzed using flow cytometry. The population of activated (CD8⁺CD25⁺) T cells were counted for each group, providing a quantifiable metric of T cell suppression. FIGS. 15A-15B display the flow cytometry readouts of engineered exosomes compared to unmodified exosomes and demonstrates significant suppression of T cell activity that is reproducible in 3 separate blood donors. Engineered exosomes were shown to suppress activated T cell activity compared to unmodified exosomes in multiple donors. FIG. 15A shows flow cytometry readouts that display the percentage of activated T cells as defined by the presence of both CD8 and CD25 present in the analyzed cell population in the top right corner of each graph. Unmodified exosome shows 37.0%, Donor 1 Engineered Exosome shows 16.6%, Donor 2 Engineered Exosome shows 21.5%, and Donor 3 Engineered Exosome shows 20.5% activated T cells. FIG. 15B shows percentages of T Cell activation. Significance was determined using the Student's t test (p<0.0001).

### 2. Inflammatory Macrophage Inhibition

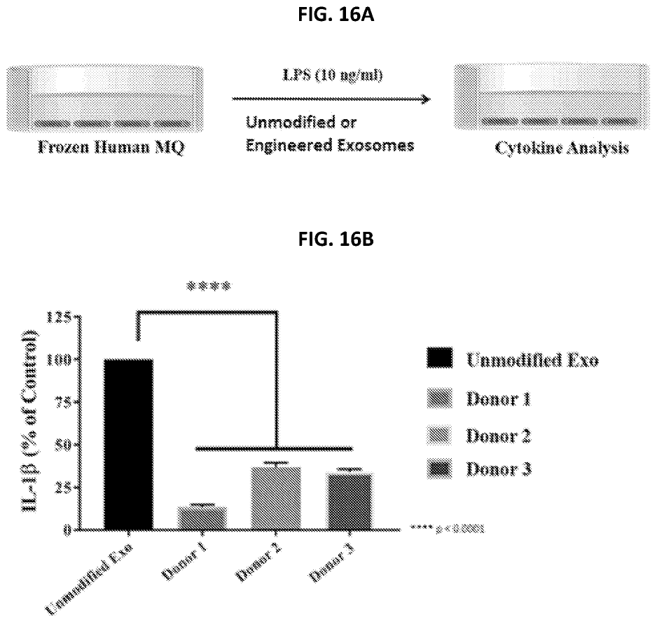

To evaluate the biological activity of engineered exosomes, human cell assays were developed to measure inflammatory macrophage inhibition using cytokine analysis. Human macrophages were thawed and activated with LPS to display an inflammatory phenotype. Inflammatory macrophages were co-cultured with engineered exosomes, unmodified exosomes (negative control), PBS (vehicle control), or free-floating passenger protein (protein control). Following the co-culture, culture media was collected and the levels of inflammatory cytokine IL-1β were measured. FIG. 16A displays a schematic representation of the assay performed. FIG. 16B depicts levels of IL-1β that were measured using unmodified exosomes as compared to engineered exosomes, and demonstrates significant inhibition of inflammatory macrophage activity that is reproducible in 3 separate blood donors. Levels of IL-1β measured were normalized against the unmodified exosome (negative control) group and displayed in a bar graph below, and significance was determined using the Student's t test (p<0.0001).

As demonstrated by this analysis, engineered exosomes significantly suppress inflammatory macrophage activity compared to unmodified exosomes in multiple donors.

### Example 9: Ligand Density Optimization

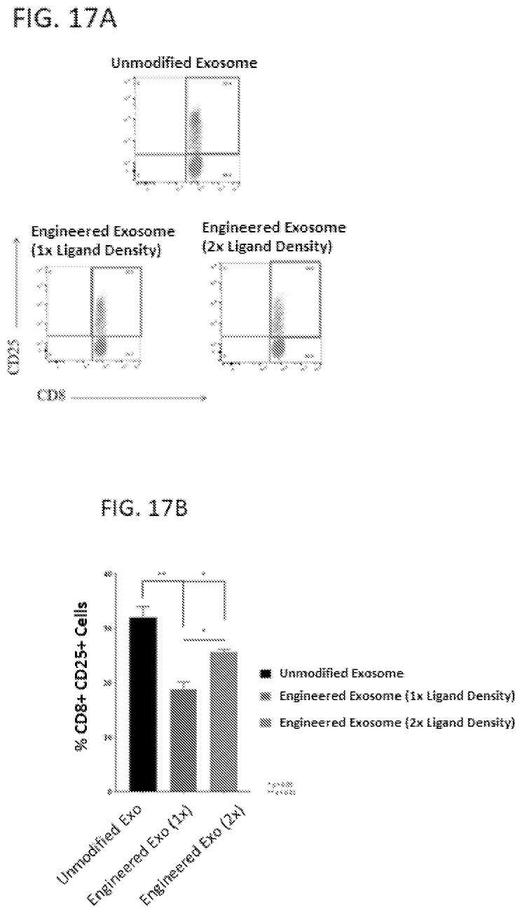

The affinity tag-receiver protein loading system disclosed herein offers several unique advantages over traditional exosome engineering methods. In addition to the ability to load multiple passenger proteins (including large proteins, antibody fragments, and secreted growth factors) onto the surface of a single exosome, the affinity tag-receiver system can also be used to control the amount of each passenger protein loaded, or the density of each passenger protein on the exosomal surface. It was hypothesized that a higher ligand density would correlate with improved biological activity. To test this, exosomes expressing the affinity receiver were mixed with varying levels of PD-L1 passenger proteins having the affinity tag to produce exosomes with different ligand surface densities. The function of these groups was evaluated using T cell suppression assays described above. FIGS. 17A-17B demonstrate that the biological activity of these exosomes is dependent on the surface density of the passenger proteins. FIG. 17A shows flow cytometry readouts that display the percentage of activated T cells as defined by the presence of both CD8 and CD25 present in the analyzed cell population in the top right corner of each graph. Unmodified exosome shows 33.4%, engineered exosome (1× ligand density) shows 25.3%, and engineered exosome (2× ligand density) shows 18.0% activate T cells. FIG. 17B shows the percentage of activated T cells in a bar graph. Significance was determined using the Student's t test. 1× Ligand Density group were loaded with 10 molecules/exosome. 2× Ligand Density group were loaded with 25 molecules/exosome.

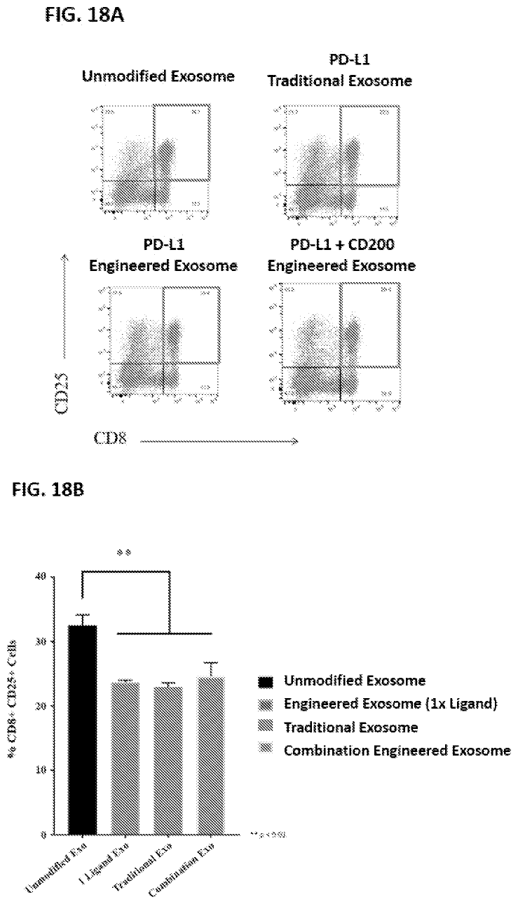

It was determined experimentally that past a certain threshold ligand density, additional passenger proteins do not improve function. This is a significant discovery because a lower density of one passenger protein leaves space on the exosomal surface for additional passenger proteins that have alternate functions, enabling the creation of a more efficient exosome-based therapeutic. Exosomes expressing the affinity receiver were then mixed with PD-L1 and CD200 passenger proteins having the affinity tag to produce exosomes with both ligands on their surface. FIGS. 18A-18B illustrate that there is no significant difference in the T cell suppressing biological activity of combinatorial exosomes displaying both ligands (PD-L1 and CD200) when compared to that of exosomes fully loaded with PD-L1. FIG. 18A shows flow cytometry readouts that display the percentage of activated T cells as defined by the presence of both CD8 and CD25 present in the analyzed cell population in the top right corner of each graph. Unmodified exosome shows 34.1%, PD-L1 traditional exosome shows 22.5%, PD-L1 engineered exosome shows 23.4%, and PD-L1+CD200 engineered exosome shows 23.1% activated T cells. FIG. 18B shows the percentage of activated T cells. Significance was determined using the Student's t test. 1 Ligand Exosomes were fully loaded with PD-L1, and Traditional Exosomes were engineered using traditional engineering methods. Combination Exosomes express both PD-L1 and CD200.

With regard to exact ligand densities used, it is difficult to measure due to the small size of the particles. In order to set up the experiments, calculations were done to equate the number of passenger protein molecules having the affinity tag to the number of exosomes expressing the anchor protein having the affinity receiver. Some key density values studied are as follows:
10 PD-L1 Molecules/Exosome→Lowest density studied, low biological activity
25 PD-L1 Molecules/Exosome→Moderate biological activity
50 PD-L1 Molecules/Exosome→Minimum threshold for maximum observed biological activity
100 PD-L1 Molecules/Exosome→Maximum observed biological activity
500 PD-L1 Molecules/Exosome→Maximum observed biological activity
50 PD-L1 Molecules+40 CD200 Molecules/Exosome→Optimized densities for optimal performance in both T cell suppression and inflammatory macrophage inhibition assays.

What is claimed is:

1. An extracellular vesicle, comprising:
a bi-layer lipid membrane comprising an inner layer and an outer layer; and
a first recombinant protein comprising i) a first transmembrane moiety embedded between the inner layer and the outer layer of the lipid membrane, and ii) a first affinity receiver located on or outside the outer layer of the lipid membrane.

2. The extracellular vesicle of claim 1, wherein the extracellular vesicle is an exosome.

3. The extracellular vesicle of claim 1, wherein the first transmembrane moiety comprises at least a portion of an exosome specific protein.

4. The extracellular vesicle of claim 1, wherein the first affinity receiver comprises a peptide or derivative thereof that is configured to bind to an affinity tag peptide or derivative thereof.

5. The extracellular vesicle of claim 1, wherein the first transmembrane moiety comprises at least a portion of IGSF8, LAMP2, CD63, CD81, PTGFR, or a functional derivative thereof.

6. The extracellular vesicle of claim 1, wherein the first transmembrane moiety comprises a peptide sequence that is at least 50% homologous to IGSF8, LAMP2, CD63, CD81, or PTGFR.

7. The extracellular vesicle of claim 1, wherein the first transmembrane moiety comprises a peptide sequence that is at least 60% homologous to IGSF8, LAMP2, CD63, CD81, or PTGFR.

8. The extracellular vesicle of claim 1, wherein the first transmembrane moiety comprises a peptide sequence that is at least 70% homologous to IGSF8, LAMP2, CD63, CD81, or PTGFR.

9. The extracellular vesicle of claim 1, wherein the first transmembrane moiety comprises a peptide sequence that is at least 80% homologous to IGSF8, LAMP2, CD63, CD81, or PTGFR.

10. The extracellular vesicle of claim 1, wherein the first transmembrane moiety comprises a peptide sequence that is at least 90% homologous to IGSF8, LAMP2, CD63, CD81, or PTGFR.

11. The extracellular vesicle of claim 1, wherein the first transmembrane moiety comprises a peptide sequence that is at least 95% homologous to IGSF8, LAMP2, CD63, CD81, or PTGFR.

12. The extracellular vesicle of claim 1, wherein the extracellular vesicle further comprises a second recombinant protein comprising 1) a second transmembrane moiety embedded between the inner layer and the outer layer of the lipid membrane, and 2) a second affinity receiver located on or outside the outer layer of the lipid membrane.

13. The extracellular vesicle of claim 6, wherein the second transmembrane moiety is the same as the first transmembrane moiety, but the second affinity receiver is different from the first affinity receiver.

14. The extracellular vesicle of claim 6, wherein the second transmembrane moiety is different from the first transmembrane moiety, but the second affinity receiver is the same as the first affinity receiver.

15. The extracellular vesicle of claim 6, wherein the second transmembrane moiety is the different from the first transmembrane moiety, and the second affinity receiver is different from the first affinity receiver.

16. The extracellular vesicle of any one of claims 6 to 8, further comprising a third recombinant protein comprising 1) a third transmembrane moiety embedded between the inner layer and the outer layer of the membrane, and 2) a third affinity receiver located on or outside the outer layer of the membrane.

17. The extracellular vesicle of claim 16, wherein the third transmembrane moiety is the different from the first transmembrane moiety, and the third affinity receiver is different from the first affinity receiver.

18. The extracellular vesicle of claim 16, wherein the third transmembrane moiety is the same as the first transmembrane moiety, and the third affinity receiver is different from the first affinity receiver.

19. The extracellular vesicle of claim 16, wherein the third transmembrane moiety is different from the first transmembrane moiety, and the third affinity receiver is the same as the first affinity receiver.

20. The extracellular vesicle of claim 1, wherein the first affinity receiver comprises a peptide that is at least 50% homologous to the amino acid sequence ID No. 20.

21. The extracellular vesicle of claim 1, wherein the first affinity receiver comprises a peptide that is at least 60% homologous to the amino acid sequence ID No. 20.

22. The extracellular vesicle of claim 1, wherein the first affinity receiver comprises a peptide that is at least 70% homologous to the amino acid sequence ID No. 20.

23. The extracellular vesicle of claim 1, wherein the first affinity receiver comprises a peptide that is at least 80% homologous to the amino acid sequence ID No. 20.

24. The extracellular vesicle of claim 1, wherein the first affinity receiver comprises a peptide that is at least 90% homologous to the amino acid sequence ID No. 20.

25. The extracellular vesicle of claim 1, wherein the first affinity receiver comprises a peptide that is at least 95% homologous to the amino acid sequence ID No. 20.

26. The extracellular vesicle of claim 1, wherein the first affinity receiver comprises a peptide that is at least 99% homologous to the amino acid sequence ID No. 20.

27. The extracellular vesicle of claim 1, wherein the first affinity receiver comprises a peptide having the amino acid sequence ID No. 20.

28. The extracellular vesicle of claim 1, wherein the first affinity receiver comprises a peptide that is at least 50% homologous to SEQ ID No: 21.

29. The extracellular vesicle of claim 1, wherein the first affinity receiver comprises a peptide that is at least 60% homologous to SEQ ID No: 21.

30. The extracellular vesicle of claim 1, wherein the first affinity receiver comprises a peptide that is at least 70% homologous to SEQ ID NO: 21.

31. The extracellular vesicle of claim 1, wherein the first affinity receiver comprises a peptide that is at least 80% homologous to SEQ ID NO: 21.

32. The extracellular vesicle of claim 1, wherein the first affinity receiver comprises a peptide that is at least 90% homologous to SEQ ID NO: 21.

33. The extracellular vesicle of claim 1, wherein the first affinity receiver comprises a peptide that is at least 95% homologous to SEQ ID NO: 21.

34. The extracellular vesicle of claim 1, wherein the first affinity receiver comprises a peptide that is at least 99% homologous to SEQ ID NO: 21.

35. The extracellular vesicle of claim 1, wherein the first affinity receiver comprises a peptide having SEQ ID NO: 21.

36. The extracellular vesicle of claim 1, further comprising a first messenger protein.

37. The extracellular vesicle of claim 35, wherein the first messenger protein comprises PD-L1.

38. The extracellular vesicle of claim 37, wherein PD-L1 is an extracellular domain of PD-L1.

39. The extracellular vesicle of claim 38, wherein the extracellular domain of PD-L1 is linked to the affinity tag.

40. The extracellular vesicle of any one of claims 37-39, further comprising a second messenger protein.

41. The extracellular vesicle of claim 40, wherein the second messenger protein comprises CD200.

42. The extracellular vesicle of claim 41, wherein CD200 is an extracellular domain of CD200.

43. The extracellular vesicle of claim 42, wherein the extracellular domain of CD200 is linked to a second affinity tag.

44. The extracellular vesicle of claim 1, further comprising a first messenger protein and a second messenger protein.

45. The extracellular vesicle of claim 44, wherein the first and second messenger proteins are immune checkpoint moieties.

46. The extracellular vesicle of any one of claim 44 or 45, wherein the first messenger protein is PD-L1 and the second messenger protein is CD200.

47. The extracellular vesicle of any one of claims 44-46, wherein the first messenger protein is an extracellular domain of PD-L1 and the second messenger protein is an extracellular domain of CD200.

48. The extracellular vesicle of any one of claim 46 or 47 for the treatment of ocular graft versus host disease.

49. The extracellular vesicle of claim 44, wherein the first messenger protein is an immune checkpoint moiety and the second messenger protein is a growth factor.

50. The extracellular vesicle of claim 49, wherein the first messenger protein is PD-L1 and the second messenger protein is BDNF.

51. The extracellular vesicle of claim 44, wherein the first messenger protein comprises CD200.

52. The extracellular vesicle of claim 51, wherein the CD200 is an extracellular domain of CD200.

53. The extracellular vesicle of any one of claims 51-52, wherein the second messenger protein comprises BDNF.

54. The extracellular vesicle of claim 53, wherein the BDNF is an extracellular domain of BDNF.

55. The extracellular vesicle of any one of claims 51-52, wherein the second messenger protein comprises PD-L1.

56. The extracellular vesicle of claim 55, wherein the PD-L1 is an extracellular domain of PD-L1.

57. The extracellular vesicle of claim 50 for the treatment of multiple sclerosis.

58. A complex, comprising,
the extracellular vesicle in claim 1, and
an affinity tag bound to the first affinity receiver of the first recombinant protein, wherein
the affinity tag is portion of a fusion protein comprising a first portion comprising the affinity tag and a second portion comprising a messenger protein.

59. The complex in claim 58, wherein the messenger protein comprises an immune checkpoint moiety.

60. The complex in claim 59, wherein the messenger protein comprises an inhibitory immune checkpoint molecule.

61. The complex in claim 59, wherein the messenger protein comprises a stimulatory immune checkpoint molecule.

62. The complex in claim 58, wherein the messenger protein comprises an immune checkpoint moiety.

63. The complex in claim 58, wherein the messenger protein comprises a growth factor.

64. The complex in claim 62, wherein the growth factor comprises BDNF.

65. The complex in claim 62, wherein the growth factor comprises GDNF.

66. The complex in claim 58, wherein the messenger protein comprises PD-L1.

67. The complex in claim 58, wherein the messenger protein comprises CD200.

68. The complex of claim 66, wherein the extracellular vesicle comprises a second messenger protein.

69. The complex of claim 68, wherein the second messenger protein comprises CD200.

70. The complex of claim 69, wherein the CD200 is an extracellular domain of CD200.

71. The complex of claim 58, wherein the second portion is an extracellular domain of an immune checkpoint moiety.

72. The complex in claim 58, wherein the affinity tag comprises a peptide having an amino acid sequence that is configured to bind to a portion of the affinity receiver.

73. The complex in claim 58, wherein the affinity tag binds to an affinity receiver through a covalent bond.

74. The complex in claim 58, wherein the affinity tag binds to an affinity receiver through an iso-peptide bond.

75. The complex in claim 58, wherein the affinity tag binds to an affinity receiver through a di-sulfide bond.

76. A method of delivering a messenger protein, comprising,
creating a fusion protein comprising the messenger protein and an affinity tag;
adding the fusion protein to the extracellular vesicle in claim 1, wherein the affinity tag binds to the first affinity receiver of the first recombinant protein.

77. A method of treating a disease in a patient, comprising administering to the patient a therapeutically effective amount of a pharmaceutical composition comprising the complex in claim 58.

78. The method in claim 77, wherein the disease is an autoimmune disease.

79. The method in claim 77, wherein the disease is a neurological disease.

80. The complex of claim 66, wherein PD-L1 is an extracellular domain of PD-L1.

81. A method of treatment of ocular graft versus host disease in a subject in need thereof, comprising administering a therapeutically effective amount of the extracellular vesicle of any one of claims 1-50.

82. A method of treatment of multiple sclerosis in a subject in need thereof, comprising administering a therapeutically effective amount of the extracellular vesicle of any one of claims 1-50.
